(19) United States
(12) Patent Application Publication
(10) Pub. No.: US 2026/0306527 A1
(43) Pub. Date: Oct. 8, 2026

(54) PRMT INHIBITOR COMPOUNDS AND THE USES THEREOF

(71) Applicant: Purdue Research Foundation, West Lafayette, IN (US)

(72) Inventors: Rong HUANG, West Lafayette, IN (US); Youchao DENG, Changsha,Hunan (CN)

(21) Appl. No.: 19/479,590

(22) PCT Filed: May 3, 2024
(86) PCT No.: PCT/US2024/027867

§ 371 Date: Oct. 28, 2025

Related U.S. Application Data
(60) Provisional application No. 63/463,867, filed on May 3, 2023.

(51) Int. Cl. A61K 31/7076; A61K 31/52; C07D 473/34; C07H 19/167

(52) U.S. Cl. CPC A61K 31/7076; A61K 31/52; C07D 473/34; C07H 19/167

(57) ABSTRACT

The present invention relates to compounds for inhibiting PRMT activity, methods of treatment for conditions exhibiting abnormal or dysfunctional PRMT activity. The invention described herein also pertains to pharmaceutical compositions and methods for treating diseases in mammals using those compounds disclosed herein.

### CROSS-REFERENCE TO RELATED APPLICATIONS

This application claims the benefit of U.S. Provisional Appl. No. 63/463,867, filed May 3, 2023, which is incorporated by reference as if fully set forth herein.

### TECHNICAL FIELD

The present disclosure relates to protein arginine methyltransferase (PRMT) inhibitors. The disclosure described herein also pertains to pharmaceutical compositions and methods for treating diseases in mammals using compounds disclosed herein.

### BACKGROUND

This section introduces aspects that may help facilitate a better understanding of the disclosure. Accordingly, these statements are to be read in this light and are not to be understood as admissions about what is or is not prior art.

Protein arginine methyltransferases (PRMTs) are attractive targets for developing therapeutic agents, but selective PRMT inhibitors targeting the cofactor S-adenosyl-L-methionine (SAM) binding site are limited. Herein, the discovery of a noncanonical but less polar SAH surrogate is reported, 2-(3-benzylureido)ethyl)thioadenosine (YD1113) (Deng et al., Acta Pharmaceutica Sinica B. 13, 4893-4905, which is incorporated by reference as fully set forth herein), by modifying previously reported pan-PRMT inhibitor II757 (Iyamu et al. 2021). YD1113 potently and selectively inhibits PRMT3/4/5 with IC₅₀ values ranging from 138 to 351 nM. Moreover, co-crystal structures illustrate that the benzyl urea moiety of YD1113 induces a unique and novel hydrophobic binding pocket in PRMTs, providing a structural basis for the selective inhibition of PRMTs by YD1113. To enhance the potency and selectivity, a substrate mimic into YD1113 was introduced to produce YD1290, forming a “T-shaped” bisubstrate analogue to engage both the SAM and substrate binding pockets. YD1290 exhibited potent and selective inhibition to type I PRMTs with IC₅₀ values less than 5 nM. In summary, the experiments described herein demonstrated the promise of YD1113 as a general SAH mimic to build potent and selective inhibitors of PRMTs. Given the similar binding mode of SAH to the Rossmann fold methyltransferases, it is rational to speculate that this unconventional SAH mimic may be adopted for other class I methyltransferases.

Protein arginine methyltransferases (PRMTs) transfer the methyl group from the cofactor S-adenosyl-L-methionine (SAM) to the guanidine group on arginine residues while generating S-adenosyl-L-homocysteine (SAH) and methylated proteins. There are three types of PRMTs according to their methylation products. Type I (PRMT 1, 2, 3, 4, 6, and 8) catalyze mono- and asymmetric di-methylation on arginine. Type II (PRMT 5 and 9) produce mono- and symmetric di-methylation on arginine. Type III (PRMT 7) only monomethylates arginine. (Zurita-Lopez et al. 2012) Abnormal expression or activity of PRMTs has been involved in various diseases, including cancers, cardiovascular diseases, inflammatory diseases, and diabetes. (Yang et al. 2013; Krause et al. 2007; Bedford et al. 2009; Blanc et al. 2017; Al-Hamashi et al. 2020) SAH has been pursued as a lead compound to build potent inhibitors for methyltransferases. Not surprisingly, selectivity is a central challenge for SAH mimics. Nevertheless, several PRMT inhibitors of SAH or sinefungin analogues have been successfully developed (FIG. 1). For instance, a PRMT5 inhibitor JNJ-64619178 is currently in clinical trials for patients with advanced solid tumors, non-Hodgkin's lymphoma, and lower-risk myelodysplastic syndrome. (Brehmer et al. 2021) SGC8158 is a selective PRMT7 inhibitor. (Szewczyk et al 2020) Recently, a PRMT pan-inhibitor II757 was developed by connecting the adenosine and guanidine moieties. (lyamu et al. 2021) PRMT inhibitors that connect 5′-thioadenosine with a tripeptide through a substituted guanidine group have been examined. (Al-Hamashi et al. 2020; Al-Hamashi et al. 2021).

However, most SAH-based PRMT inhibitors comprise 5′-thioadenosine or its mimic and a guanidine function group, losing interactions with the homocysteine binding channel. One reason is the polarity of the α-amino acid moiety causing low cellular uptake. Another reason is the challenge of identifying less polar surrogates to fit into a deep narrow binding channel while retaining the key interactions. Notably, the α-amino acid moiety (alpha-amino acid moiety) of the homocysteine forms multiple interactions with the methyltransferases and contributes significantly to the cofactor SAM binding. Therefore, the discovery of a homocysteine mimic would be beneficial to boost the potency of SAH-based inhibitors for methyltransferases. To date, two cases have been reported. One example is the PRMT4 inhibitor SKI-72, a sinefungin derivative with a benzylamine to occupy the α-amino carboxylate moiety of the SAH binding site according to the co-crystal structure (PDB ID: 6D2L). Another example is the PRMT4 bisubstrate inhibitor that bears a 2-aminopyrimidine or 2-aminopyridine, alternatively occupying either the a-amino carboxylate moiety of SAH or the substrate arginine binding site (PDB ID: 6S74, 6S7A). (Gunnell et al. 2020) Nonetheless, very few PRMT inhibitors engage the homocysteine binding channel. Notably, a PRMT 5/7 dual inhibitor DS437 was predicted to anchor in the homocysteine binding site but with no experimental evidence. (Smil et al. 2015)

The present disclosure describes compounds identified to be useful as PRMT inhibitors as shown in TABLE 1. The present disclosure provides for compounds as shown and described in the Figures.

The present disclosure relates to a compound of the formula (I):

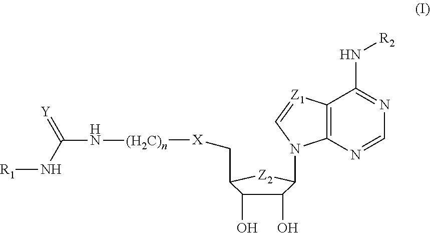

or a pharmaceutically acceptable salt thereof, wherein:
X is a N or S,
n is an integer from 1-12; and
Y is NR^{p} or O, wherein R^{p} is H or a protecting group (e.g., benzyloxycarbonyl, 9-fluorenylmethyl carbamate, acetyl, benzyl, and p-toluenesulfonyl), and
Z₁ is N or CH, and
Z₂ is O or CH₂, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl. In one embodiment, R₁ is CH₃. Alternatively, or in addition, R₂ is H. In one example, R₁ or R₂ is selected from the group consisting of:

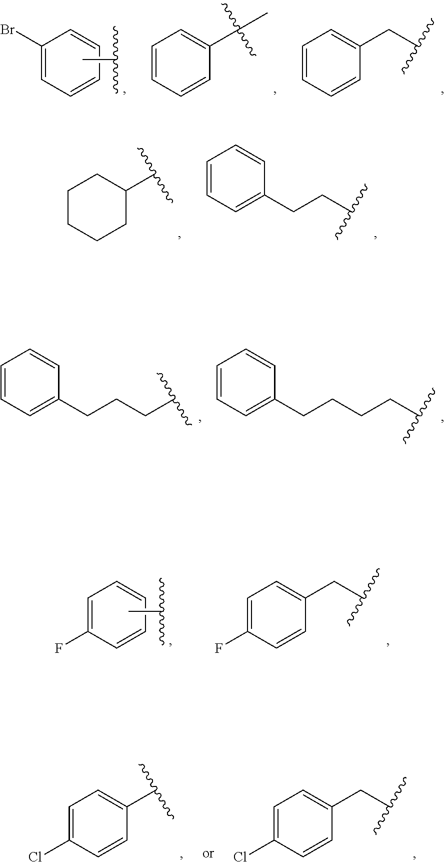

each of which can be further substituted. An example of a compound where Y is NR^{p} is:

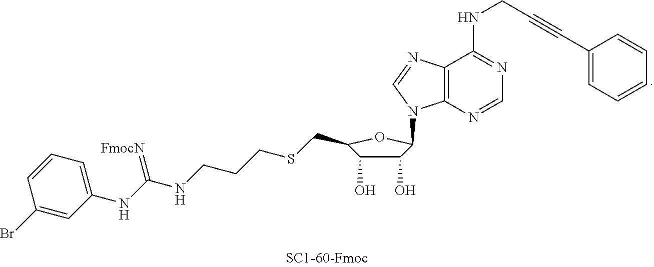

The present disclosure relates to a compound of the formula:

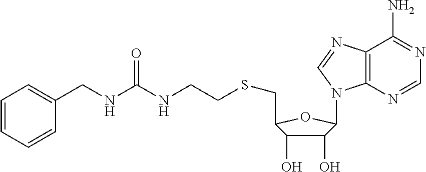 [SMILES: Nc1ncnc2c1ncn2C1OC(CSCCNC(=O)NCc2ccccc2)C(O)C1O]

or a pharmaceutically acceptable salt thereof.

The present disclosure relates to a compound of the formula (II):

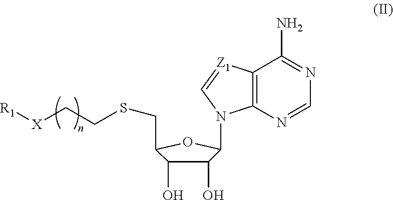

or a pharmaceutically acceptable salt thereof,
wherein:
n is an integer from 0-12,
Z₁ is N or CH, and
X is O or NH and
R₁ is an aryl or heterocyclic. R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The present disclosure relates to a compound of the formula (IIa):

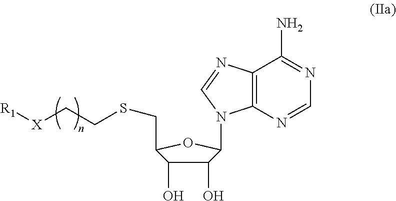

or a pharmaceutically acceptable salt thereof,
wherein:
n is an integer from 0-12,
X is O or NH and
R₁ is an aryl or heterocyclic. R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The present disclosure relates to a compound of the formula:

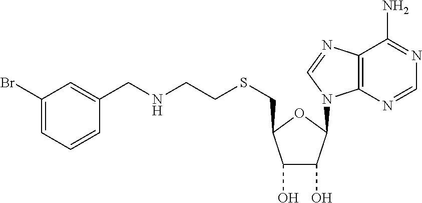 [SMILES: Nc1ncnc2c1ncn2[C@@H]1O[C@H](CSCCNCc2cccc(Br)c2)[C@@H](O)[C@H]1O]

or a pharmaceutically acceptable salt thereof.

The present disclosure relates to a compound of the formula (III):

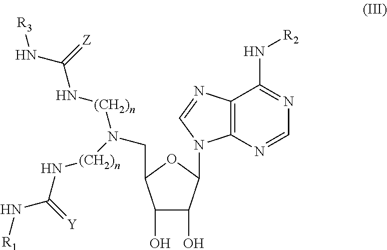

or a pharmaceutically acceptable salt thereof,
wherein:
each n is independently an integer from 1-12; and
Y and Z are either NH or O, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The present disclosure relates to a compound of the formula:

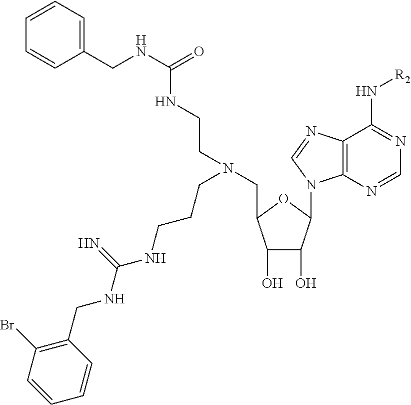

or a pharmaceutically acceptable salt thereof.

The present disclosure provides a compound of the formula (IIIa):

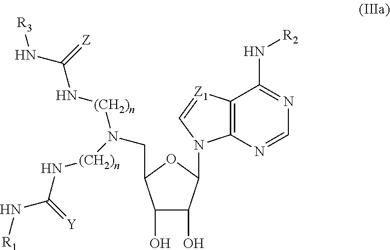

wherein:
each n is independently an integer from 1-12; and
Y and Z are either NH or O, and
Z₁ is N or CH, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

In one embodiment, the disclosure provides for a compound having the formula selected from the group consisting of:

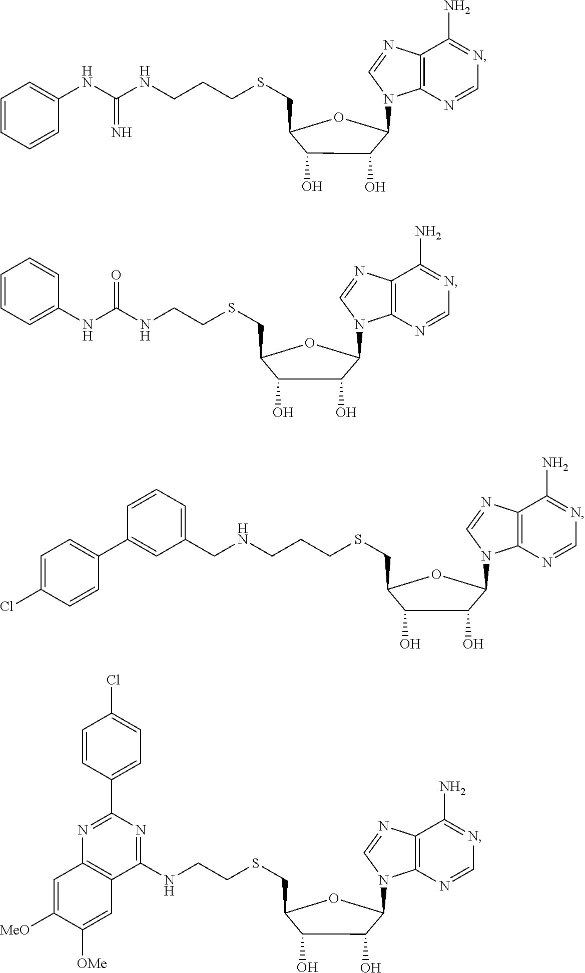 [SMILES: N=C(NCCCSC[C@H]1O[C@@H](n2cnc3c(N)ncnc32)[C@H](O)[C@@H]1O)Nc1ccccc1 ; Nc1ncnc2c1ncn2[C@@H]1O[C@H](CSCCNC(=O)Nc2ccccc2)[C@@H](O)[C@H]1O ; Nc1ncnc2c1ncn2[C@@H]1O[C@H](CSCCCNCc2cccc(-c3ccc(Cl)cc3)c2)[C@@H](O)[C@H]1O ; COc1cc2nc(-c3ccc(Cl)cc3)nc(NCCSC[C@H]3O[C@@H](n4cnc5c(N)ncnc54)[C@H](O)[C@@H]3O)c2cc1OC]

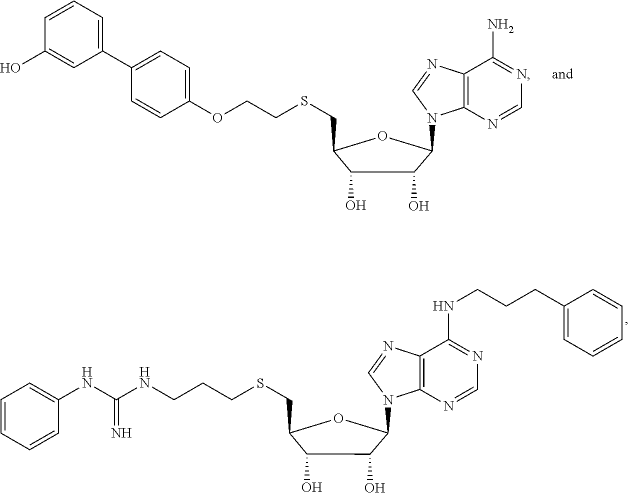 [SMILES: Nc1ncnc2c1ncn2[C@@H]1O[C@H](CSCCOc2ccc(-c3cccc(O)c3)cc2)[C@@H](O)[C@H]1O ; N=C(NCCCSC[C@H]1O[C@@H](n2cnc3c(NCCCc4ccccc4)ncnc32)[C@H](O)[C@@H]1O)Nc1ccccc1]

or a pharmaceutically acceptable salt thereof.

In one embodiment, the disclosure provides for a compound selected from the group consisting of:

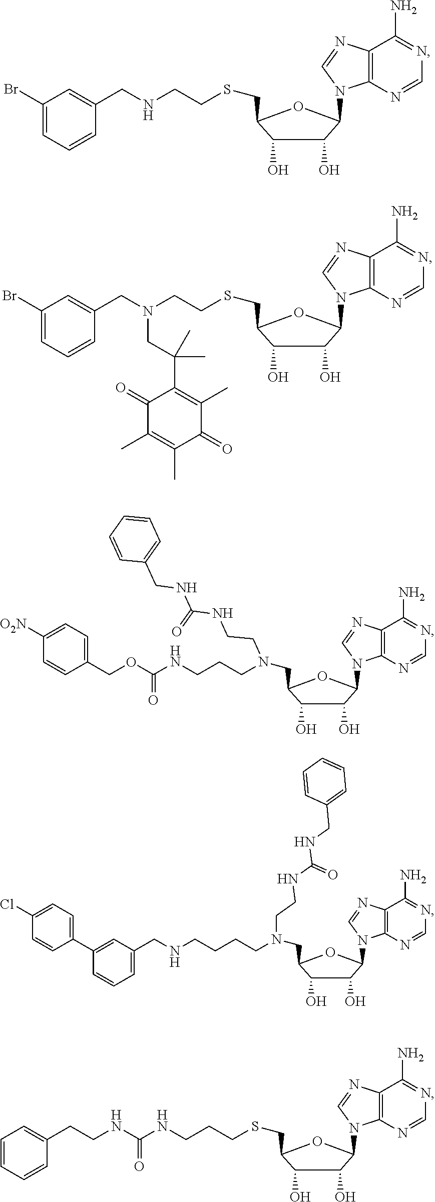

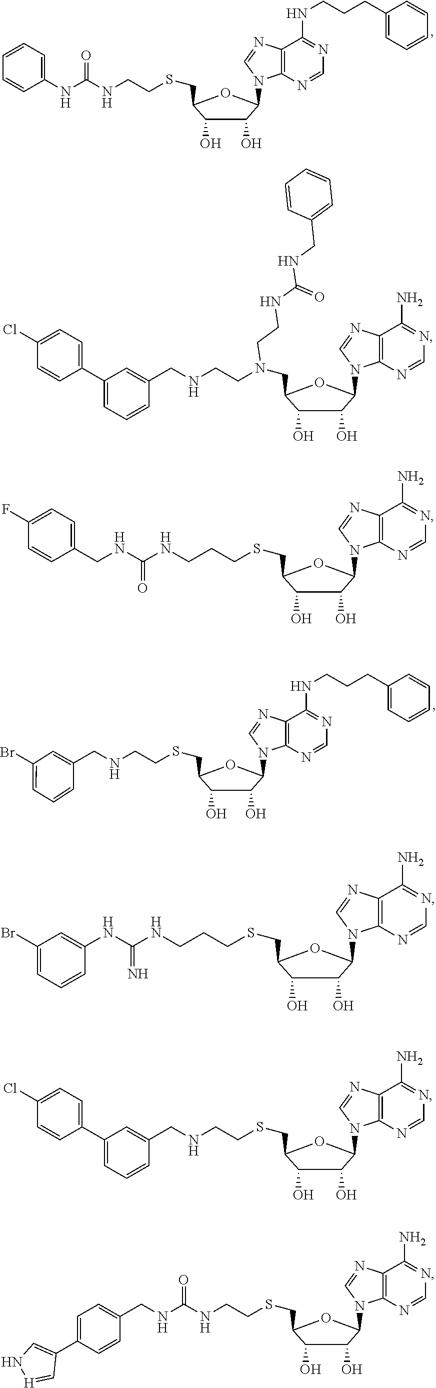

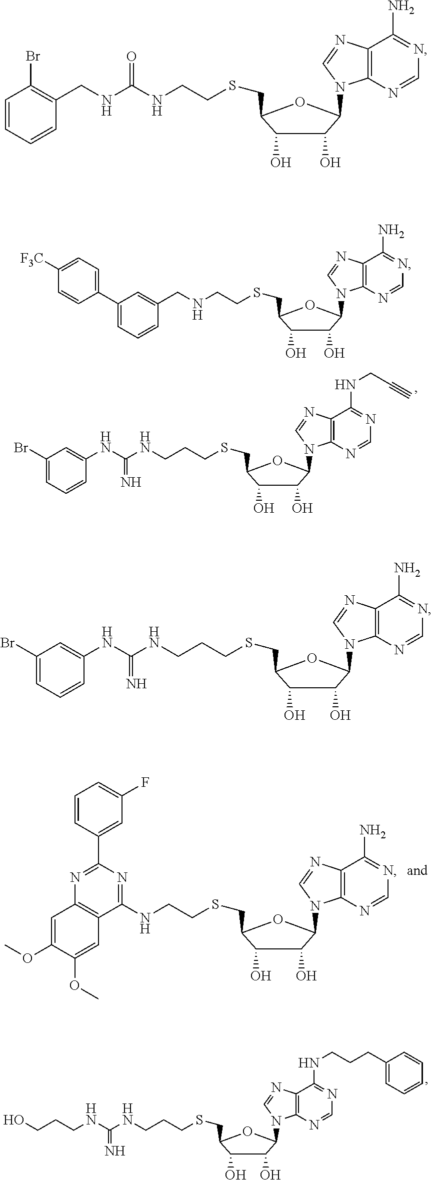

or a pharmaceutically acceptable salt thereof.

The present disclosure provides for pharmaceutical compositions comprising one or more of the described compounds, or a pharmaceutically acceptable salt thereof, together with one or more diluents, excipients or carriers.

The present disclosure provides for pharmaceutical compositions comprising one or more of the described compounds, or a pharmaceutically acceptable salt thereof, in combination with one or more other compounds by the same or different mode of action, together with one or more diluents, excipients or carriers.

The present disclosure provides for a method for inhibiting PRMT activity comprising the step of administering to a mammal in need of relief from the effects of PRMT activity a therapeutically effective amount of one or more compounds of formula (I):

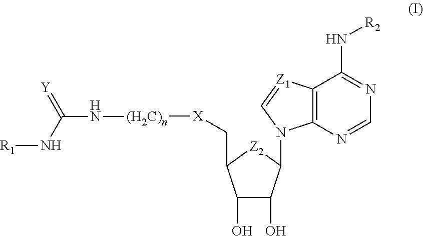

or a pharmaceutically acceptable salt thereof, wherein:
X is a N or S, and
n is an integer from 1-12; and
Y is NR^{p} or O, wherein R^{p} is H or a protecting group, and
Z₁ is N or CH, and
Z₂ is O or CH₂, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl. An example of a compound where Y is NR^{p} is:

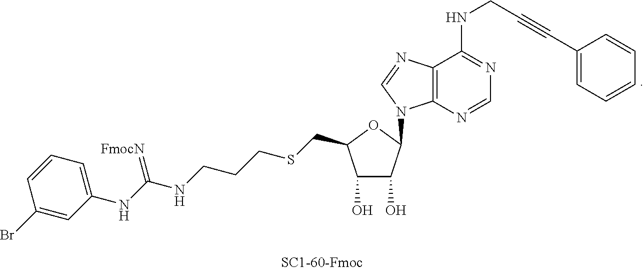

The present disclosure provides for a method for inhibiting PRMT activity comprising the step of administering to a mammal in need of relief from the effects of PRMT activity a therapeutically effective amount of one or more compounds, wherein said compound has a formula (II):

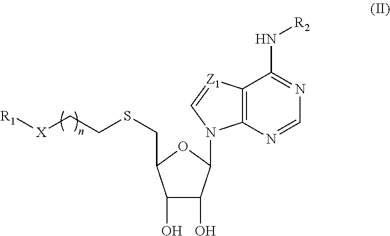

or a pharmaceutically acceptable salt thereof, wherein:
n is an integer from 0-12,
Z₁ is N or CH, and
X is O or NH and
R₁ is an aryl or heterocyclic. Or R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The present disclosure provides for a method for inhibiting PRMT activity comprising the step of administering to a mammal in need of relief from the effects of PRMT activity a therapeutically effective amount of one or more compounds, wherein the compound has a formula (IIa):

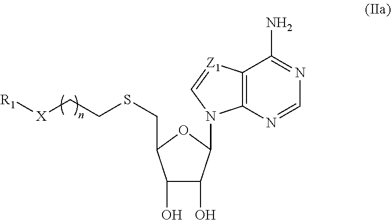

or a pharmaceutically acceptable salt thereof,
n is an integer from 0 to 12,
X is O or NH, and
R¹ is an aryl or heterocyclic.

The present disclosure provides for a method for inhibiting PRMT activity comprising the step of administering to a mammal in need of relief from the effects of PRMT activity a therapeutically effective amount of one or more compounds, wherein the compound is selected from the group consisting of:

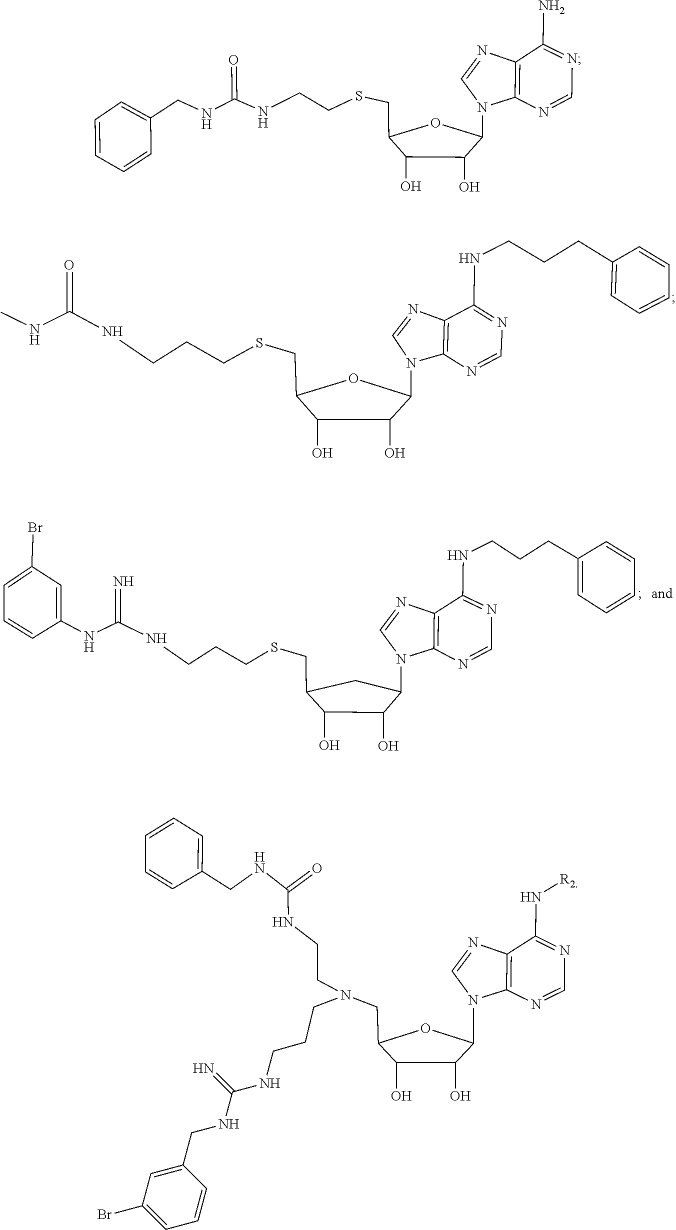

The present disclosure provides for a method for inhibiting PRMT activity comprising the step of administering to a mammal in need of relief from the effects of PRMT activity a therapeutically effective amount of one or more compounds, wherein the compound has the formula (III) and (IIIa); wherein:

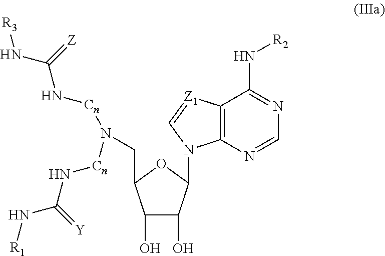

n is optionally an integer from 1-12; and
Y and Z are either NH or O, and
Z₁ is N or CH, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl,
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The present disclosure provides for a method for inhibiting PRMT activity comprising the step of administering to a mammal in need of relief from the effects of PRMT activity a therapeutically effective amount of one or more compounds of the present disclosure via medically acceptable administrative routes. Such routes include orally, intravenously, intracranially, via cerebral spinal fluid, dermally, intradermally, intramuscularly, via suppository, etc.

The present disclosure provides for administration of the compounds of the disclosure in all suitable forms of pharmaceutical packaging, including time-release, oral absorption, injection, intravenous administration, capsule, tablet, liquid suspension, liquid solution, eye drops, etc.

The present disclosure provides a method for screening for compounds as shown in FIG. 11. The present disclosure provides for chemical synthesis methods as shown in FIGS. 12 and 13.

The features and benefits of the compounds and methods of the present disclosure may be more fully illustrated in view of the Drawings and description thereof.

### DESCRIPTION OF THE DRAWINGS

The drawings illustrate generally, by way of example, but not by way of limitation, various embodiments discussed herein.

FIG. 1 illustrates some structures of the representative PRMTs inhibitors.

FIG. 2 depicts the complex structure of PRMT3 (gray cartoon) with II710 (light blue stick, PDB ID: 8G2F) or SAH (gray stick, PDB ID: 2FYT). (FIG. 2a, FIG. 2b) Detailed interactions of II710 with PRMT3 in the complex structures. A 2Fo-Fc omit map contoured at 1.0 σ is shown for II710 as a transparent turquoise isosurface. (FIG. 2c) Structure alignment of II710 and SAH. (FIG. 2d) 2D interaction diagram of SAH with PRMT3.

FIG. 3 illustrates inhibition mechanism and selectivity of YD1113. (FIG. 3a) IC₅₀ curves of YD1113 at varying concentrations of peptide substrate H4-21 with a fixed concentration of SAM; (FIG. 3b) Linear regression plot of IC₅₀ values for YD1113 with an increased ratio of peptide/Km; (FIG. 3c) IC₅₀ curves of YD1113 at varying concentrations of SAM with a fixed concentration of H4-21; (FIG. 3d) Plot of IC₅₀ values for YD1113 with an increased ratio of SAM/Km value. All the experiments were performed in triplicate (n=3) and presented as mean±SD. (FIG. 3e) Inhibition study of YD1113 on PRMT family members at 10 μM. Both SAM and substrates are at their physiological values. (FIG. 3f) IC₅₀ determination of YD1113 on PRMT3, 4, 5 and 7.

FIG. 4 illustrates X-ray co-crystal structure of PRMT3 (gray cartoon) bound with YD1113 (light blue stick, PDB ID:8G2G) or SAH (gray stick, PDB ID: 2FYT). (FIG. 4a) overview of the X-ray structure of PRMT3 with YD1113. (FIG. 4b) A 2Fo-Fc omit map contoured at 1.0 σ is shown for YD1113 as a transparent light blue isosurface. (FIG. 4c) Ligand interaction diagram of YD1113 with PRMT3. (FIG. 4d) The binding pocket of benzyl urea moiety of YD1113 (shown in the light blue mesh) in PRMT3. (FIG. 4e) Structure alignment of the key residues of the hydrophobic pocket. The residues of the SAH/YD1113-PRMT3 complex are gray and light blue sticks, respectively.

FIG. 5 illustrates X-ray co-crystal structure of PRMT4 (gray cartoon) complexed with YD1113 (light blue stick, PDB ID:8G2H) or SAH (gray stick, PDB ID: 51H3). (FIG. 5a) overview of the X-ray structure of PRMT4 bound with YD1113. (FIG. 5b) A 2Fo-Fc omit map contoured at 1.0 σ is shown for YD1113 as a transparent light blue isosurface. (FIG. 5c) Ligand interaction diagram of YD1113 with PRMT4. (FIG. 5d) The binding pocket of the benzyl urea moiety of YD1113 in PRMT4. And the binding pocket of the benzyl group was shown in the light blue mesh. (FIG. 5e) Structure alignment of the residues of the hydrophobic pocket. The residues of the SAH/YD1113-PRMT4 complex are gray and light blue sticks, respectively.

FIG. 6A illustrates formation of (FIG. 6a) A hybrid of YD1113 and II757 affords a “T-shaped” PRMT inhibitor YD1290. FIG. 6B presents data for (FIG. 6b) Inhibition of YD1290 on PRMTs in comparison with YD1113 and II757.

FIG. 7 illustrates data on the Inhibition mechanism of YD1290. (FIG. 7a, FIG. 7b) YD1290 is a SAM-site competitive inhibitor of PRMT1 as the IC₅₀ value increases linearly with the ratio of SAM/Km increasing. (FIG. 7c, FIG. 7d) YD1290 is a peptide-site competitive inhibitor of PRMT1 as the IC₅₀ value slightly increases with the ratio of peptide/Km increases.

FIG. 8 illustrates X-ray co-crystal structure of PRMT4 (gray cartoon) complexed with YD1290 (light blue stick, PDB ID: 8G21). (FIG. 8a, FIG. 8a b) Costructure and molecular interactions of YD1290 with PRMT4. (FIG. 8a c) Structure alignment of YD1290 and SAH. (FIG. 8a d) Selectivity study of YD1290 on seven inhouse methyltransferases and SAHH. A 2Fo-Fc omit map contoured to 1.0 σ is shown for YD1290 as a transparent light blue isosurface.

FIG. 9 presents a table of IC₅₀ values of synthesized urea compounds on PRMT3.

FIG. 10 shows the structures and selectivities of the representative reported PRMT inhibitors.

FIG. 11 illustrates the design of a focused library for targeting PRMTs.

FIG. 12 illustrates Synthetic Scheme I, Synthesis of analogues 6, 8, 10, and 12 (FIG. 12a) PPh₃, DIAD, AcSH, THF, 0° C.-r.t., N₂; (FIG. 12b) NaOMe, MeOH, 0° C.-r.t. (FIG. 12c) TFA, DCM, r.t.; (FIG. 12d) EDC, DIPEA, DCM/DMF, r.t.; (FIG. 12e) Piperidine, DCM, r.t.; (FIG. 12f) Pd(PPh₃)₄, K₂CO₃, Dioxane/H₂O, 125° C., 30 min, microwave; (FIG. 12g) DMF, Et₃N, r.t.; (FIG. 12h) NaBH3CN, MeOH, r.t.; (FIG. 12i) DMF/DCM, Et₃N, r.t.

FIG. 13 illustrates the Synthesis of analogues 14a-14f and 19a-19m

(FIG. 13a) MeONa, MeOH, 0° C.-r.t.; (FIG. 13b) Pd(PPh₃)₄, K₂CO₃, Dioxane/H₂O, 125° C., 30 min, microwave; (FIG. 13c) n-Butanol, reflux, 12 h; (FIG. 13d) PPh₃, DIAD, AcSH, THF, 0° C.-r.t.; (FIG. 13e) NaOMe, MeOH, 0° C.-r.t.; (FIG. 13f) TFA, DCM, r.t.; (FIG. 13g) EDC, DIPEA, DCM/DMF, r.t.; (FIG. 13h) Piperidine,

DCM, r.t.

FIG. 14 illustrates the Heat map of the profiling result for Type I PRMTs (1, 3, 4, 6 and 8). Type II PRMT5 and Type III PRMT7. Also included are DNMT1 here to show the wide application. All compounds were tested at 10 μM in the focused library.

FIG. 15 illustrates the chemical trends in primary screening. FIG. 15A. Number and type of hit compounds for each PRMT. (Hits were defined as >70% inhibition at 10 μM). FIG. 15B. Example compounds

FIG. 16 presents IC₅₀ values of representative synthesized compounds containing guanidine group on both PRMT1 and PRMT3.

FIG. 17 presents IC₅₀ values of representative synthesized compounds with N6-substitution on PRMT1.

FIG. 18 presents IC₅₀ values of representative synthesized compounds with N6-substitution on PRMT4.

FIG. 19 presents the IC₅₀ values of some top inhibitors for PRMTs.

FIGS. 20A and 20B presents structure, selectivity, and IC₅₀ values of YD1130 as a selective potent PRMT4 inhibitor.

FIG. 21A presents design of YD1342, FIG. 21B shows data for cellular inhibition, and growth inhibition of breast cancer cell line MDA-MB-231, FIG. 21C graphs the results of inhibition.

FIG. 22A presents the design, structures, and FIG. 22B data for selectivity of YD1288 as a dual selective and potent PRMT4/5 inhibitor, while FIG. 22C shows data for YD1289 as a selective and potent PRMT4 inhibitor. In addition, the structures of prodrug YD1302 (FIG. 22D) and YD1303 (FIG. 22E) are shown and data for cellular inhibition of YD1288 (FIG. 22F).

FIG. 23 presents design, structures and selectivity data of YD1305 and YD1349 as potent and selective PRMT4 inhibitors.

FIG. 24 presents design, structures for YD1-248 (FIG. 24A), YD1-250 (FIG. 24B), YD1-254 (FIG. 24C) and inhibition data for the three PRMT1 inhibitors (FIGS. 24D and 24E).

FIG. 25 is a plot showing the inhibitory effect of YD1375 on PRMT1.

FIG. 26 is a scheme for the synthesis of compound YD1375.

FIG. 27 is a scheme for the synthesis of another compound of the disclosure.

FIG. 28 are tables showing additional compounds of the disclosure and ther IC₅₀ values for PRMT1 and PRMT3.

FIG. 29 is the structures of AK438, AK442, AK447, YD1246, YD1194, YD1195, YD1231, YD1147, AK219, AK221, YD1234, YD192, YD159/11757, and YD1135 and IC₅₀ values for PRMT1, 4, 5, 6, 7, and 8.

FIG. 30 is the structures of compounds of the disclosure.

FIG. 31 is a synthetic scheme and structures of compounds of the disclosure.

FIG. 32 is the pharmacokinetic (PK) data for the compound YD1342.

FIG. 33 is the inhibition by various PRMTs by YD1130.

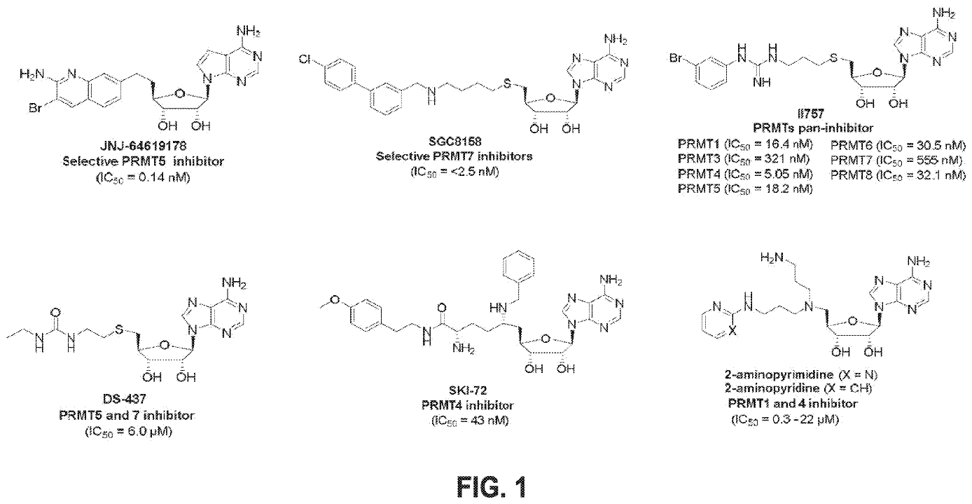

### DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

The present disclosure relates to a compound of the formula (I):

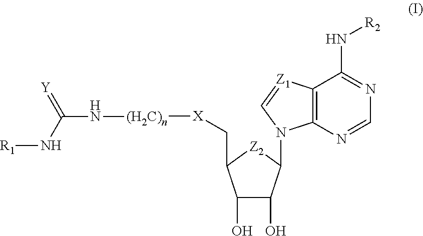

or a pharmaceutically acceptable salt thereof, wherein:
X is a N or S,
n is an integer from 1-12; and
Y is NR^{p} or O, wherein R^{p} is H or a protecting group, and
Z₁ is N or CH, and
Z₂ is O or CH₂, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl. In one embodiment, R₁ is CH₃. Alternatively, or in addition, R₂ is H. In one example, Z₂ is O. In another example, Z₂ is CH₂. Alternatively, or in addition, X is N. In other examples, X is S. In one example, Y is NH. In another example, Y is O. In one example, Z₁ is N. In one example, n is an integer from 1 to 5, 1 to 3, 2 to 3, 5 to 10, 4 to 8, or 3 to 10. In one example, R₁ or R₂ is selected from the group consisting of:

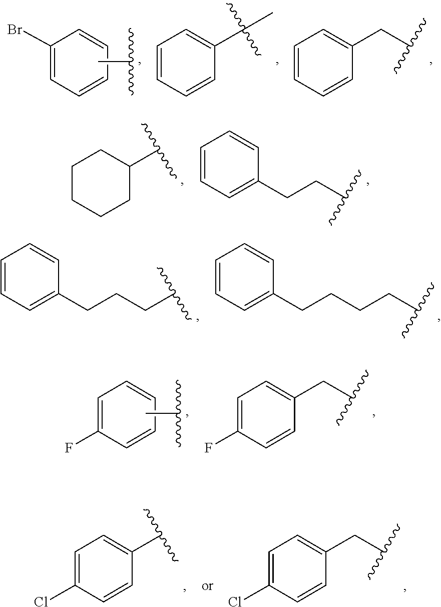

each of which can be further substituted. In one example, R₁ is:

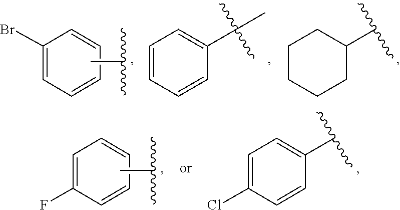

each of which can be further substituted. Alternatively, R₁ is:

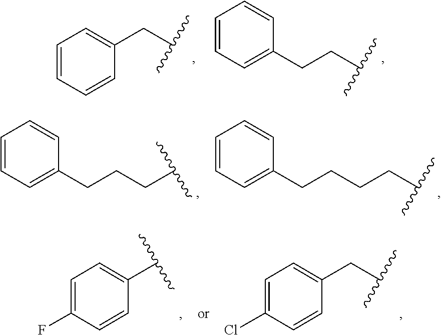

each of which can be further substituted.

The present disclosure relates to a compound of the formula:

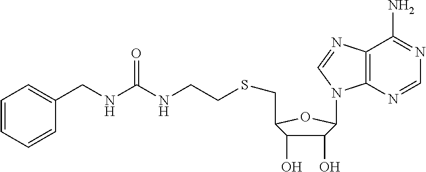 [SMILES: Nc1ncnc2c1ncn2C1OC(CSCCNC(=O)NCc2ccccc2)C(O)C1O]

or a pharmaceutically acceptable salt thereof.

The present disclosure relates to a compound of the formula (II):

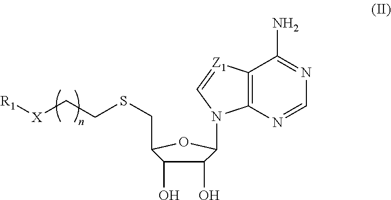

or a pharmaceutically acceptable salt thereof,
wherein:
n is an integer from 0-12,
Z₁ is N or CH, and
X is O or NH and
R₁ is an aryl or heterocyclic. R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl. In one example, n is an integer from 1 to 5, 1 to 3, 2 to 3, 5 to 10, 4 to 8, or 3 to 10. In one example, R₁ is:

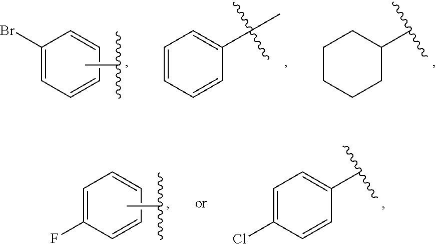

each of which can be further substituted. Alternatively, R₁ is:

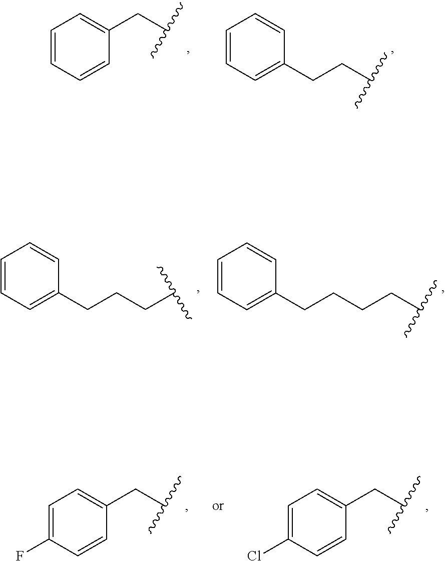

each of which can be further substituted.

The present disclosure relates to a compound of the formula (IIa):

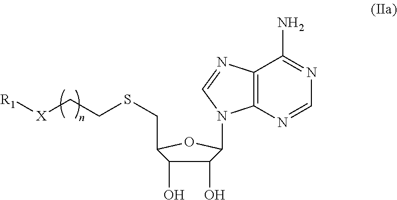

or a pharmaceutically acceptable salt thereof,
wherein:
n is an integer from 0-12,
X is O or NH and
R₁ is an aryl or heterocyclic. R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl. In one example, n is an integer from 1 to 5, 1 to 3, 2 to 3, 5 to 10, 4 to 8, or 3 to 10. In one example, R₁ is:

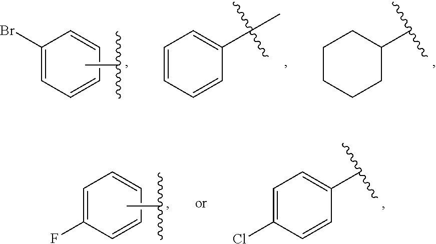

each of which can be further substituted. Alternatively, R₁ is:

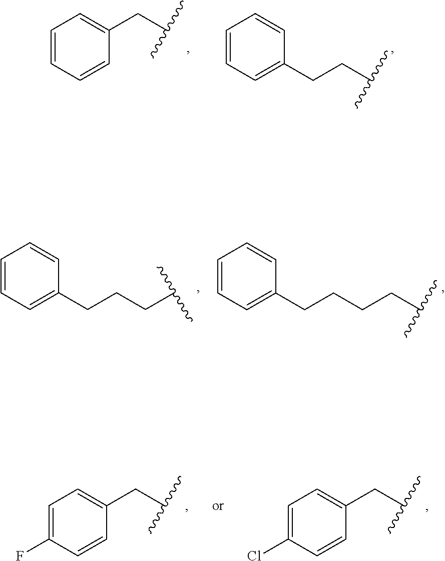

each of which can be further substituted.

The present disclosure relates to a compound of the formula:

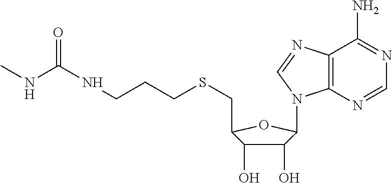 [SMILES: CNC(=O)NCCCSCC1OC(n2cnc3c(N)ncnc32)C(O)C1O]

or a pharmaceutically acceptable salt thereof.

The present disclosure relates to a compound of the formula (III):

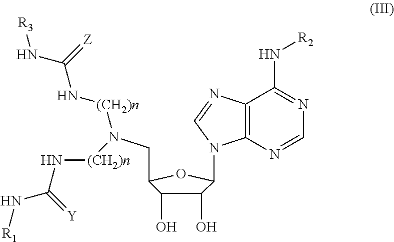

or a pharmaceutically acceptable salt thereof,
wherein:
each n is independently an integer from 1-12; and
Y and Z are either NH or O, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl. In one example, each n is, independently, an integer from 1 to 5, 1 to 3, 2 to 3, 5 to 10, 4 to 8, or 3 to 10. In one example, Y is NH. Alternatively, or in addition, Z is O. In another example, Y is O. Alternatively, or in addition, Z is NH. Alternatively, or in addition, R₁ and R₃ are each, independently:

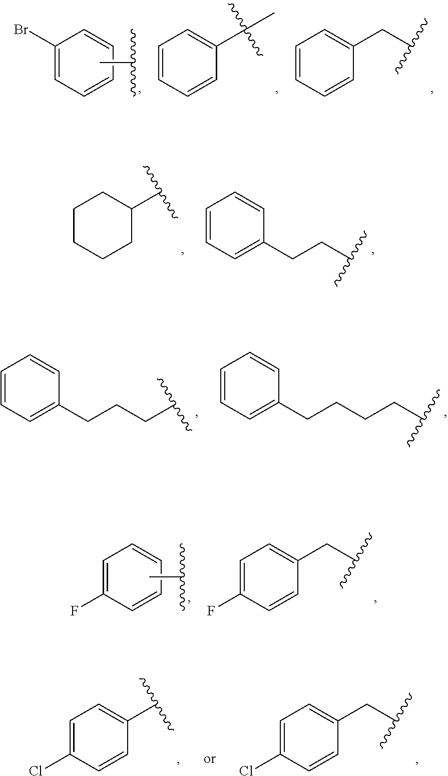

each of which can be further substituted. In one example, R₁ is:

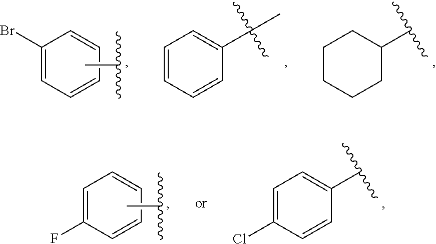

each of which can be further substituted. Alternatively, or in addition, R₃ is:

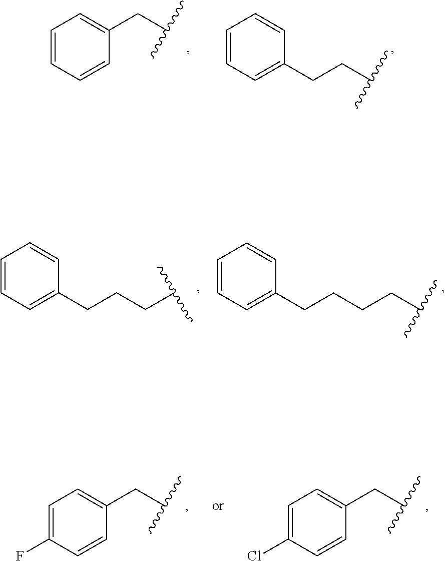

each of which can be further substituted. In another example, R₃ is:

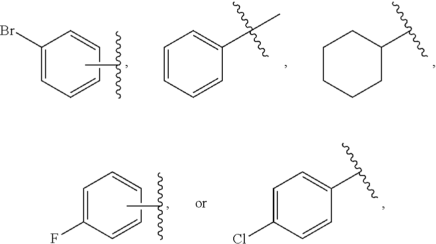

each of which can be further substituted. Alternatively, or in addition, R₁ is:

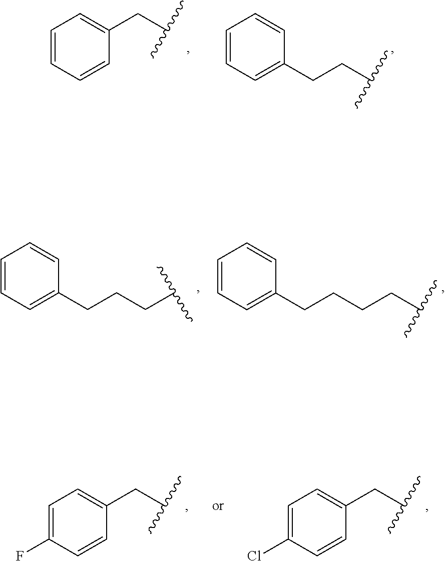

each of which can be further substituted. In another example, R₁ and R₃ are each, independently,

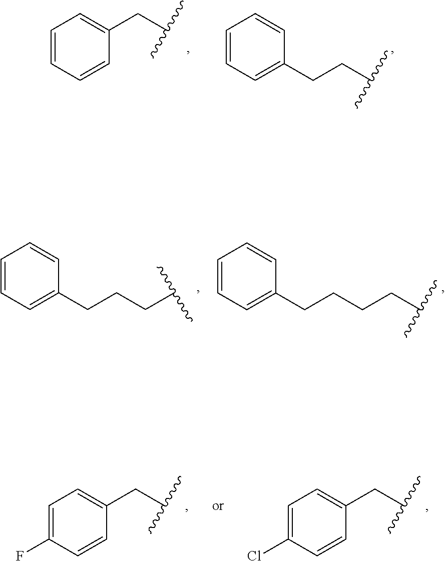

each of which can be further substituted.

The present disclosure relates to a compound of the formula:

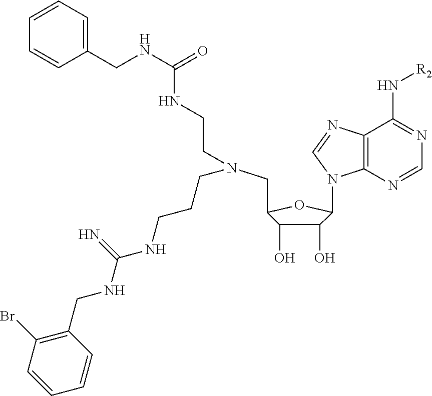

or a pharmaceutically acceptable salt thereof.

The present disclosure provides a compound of the formula (IIIa):

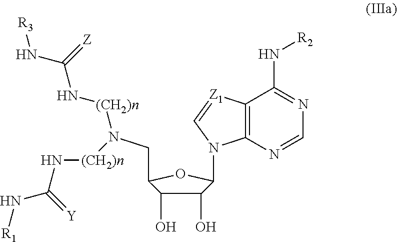

wherein
each n is independently an integer from 1-12; and
Y and Z are either NH or O, and
Z₁ is N or CH, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl. In one example, each n is, independently, an integer from 1 to 5, 1 to 3, 2 to 3, 5 to 10, 4 to 8, or 3 to 10. In one example, Y is NH. Alternatively, or in addition, Z is O. In another example, Y is O. Alternatively, or in addition, Z is NH. Alternatively, or in addition, R₁ and R₃ are each, independently:

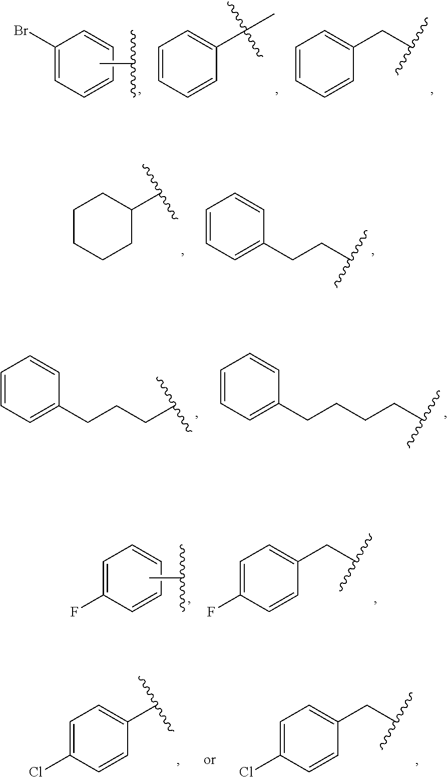

each of which can be further substituted. In one example, R₁ is:

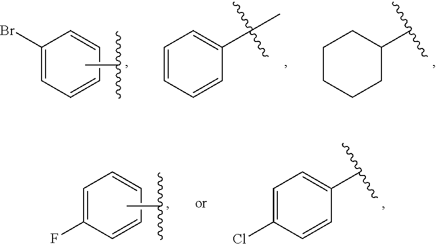

each of which can be further substituted. Alternatively, or in addition, R₃ is:

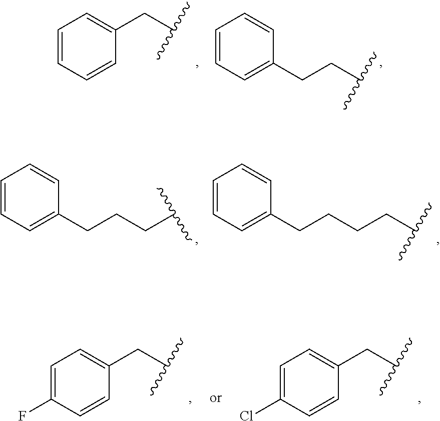

each of which can be further substituted. In another example, R₃ is:

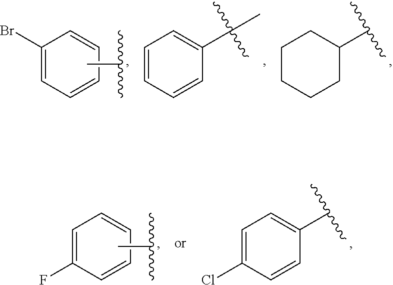

each of which can be further substituted. Alternatively, or in addition, R₁ is:

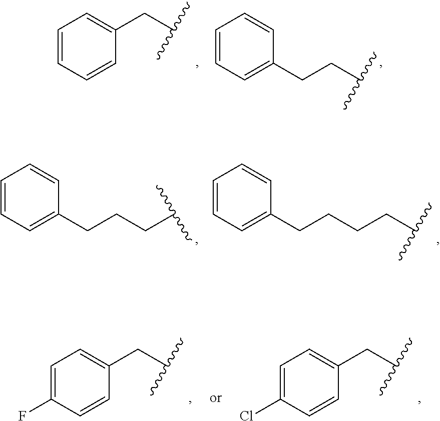

each of which can be further substituted. In another example, R₁ and R₃ are each, independently,

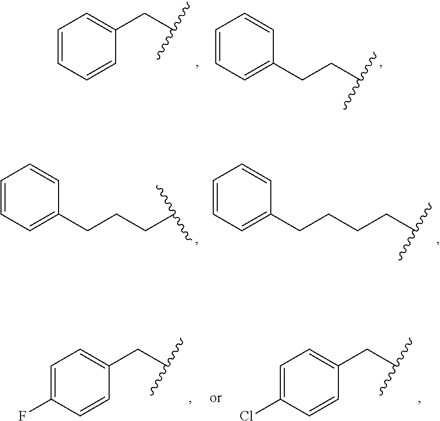

each of which can be further substituted.

In one embodiment, the disclosure provides for a compound having the formula selected from the group consisting of:

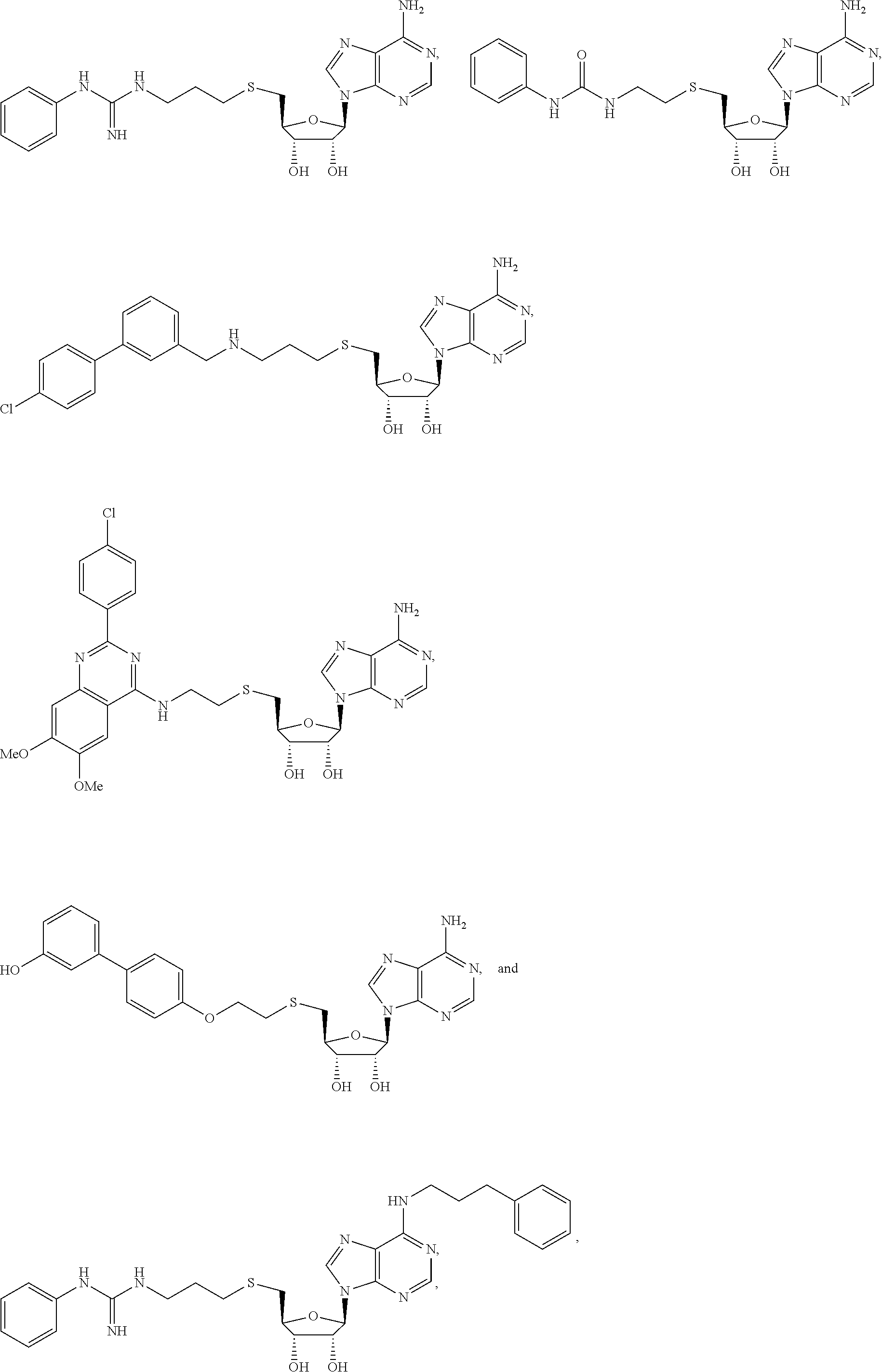

or a pharmaceutically acceptable salt thereof.

In one embodiment, the disclosure provides for a compound selected from the group consisting of:

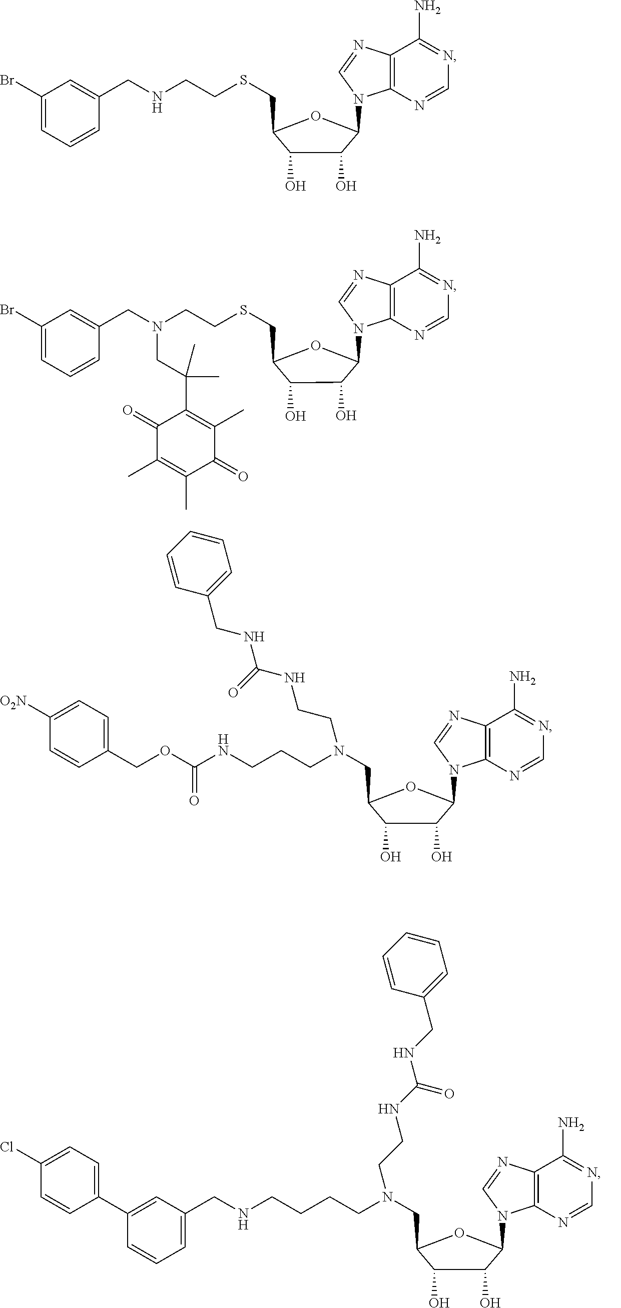

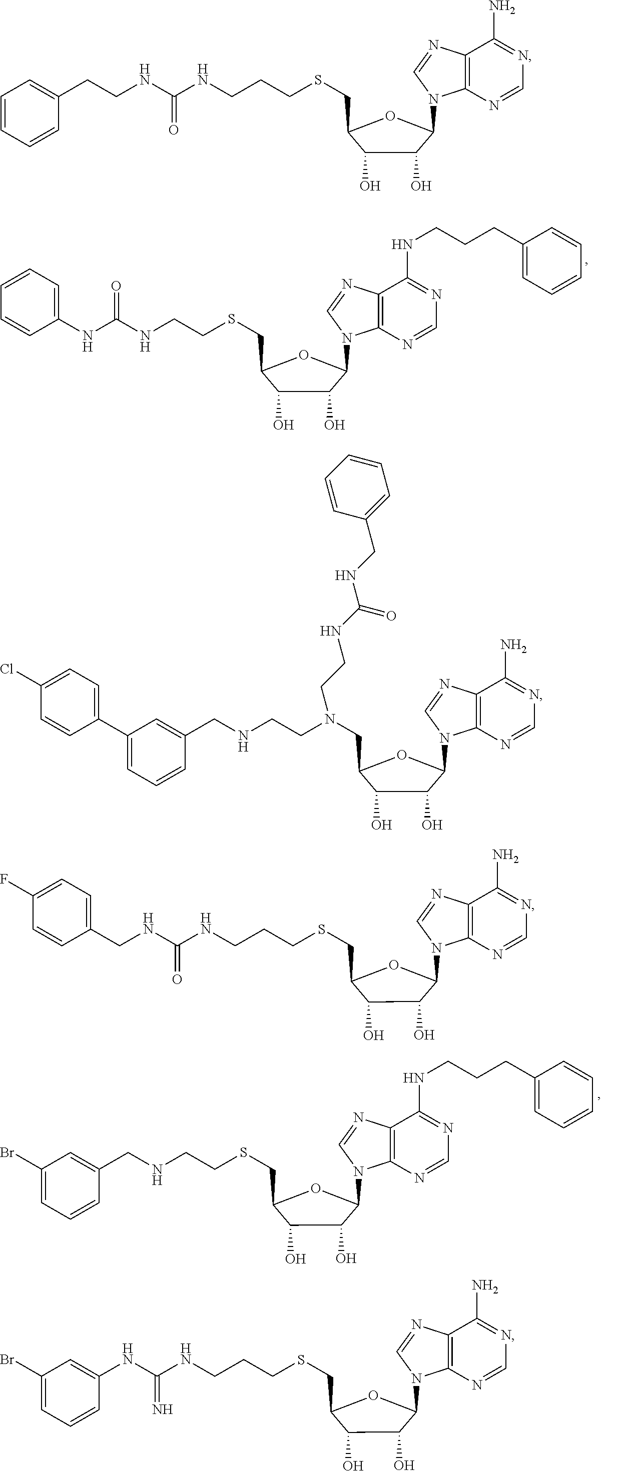

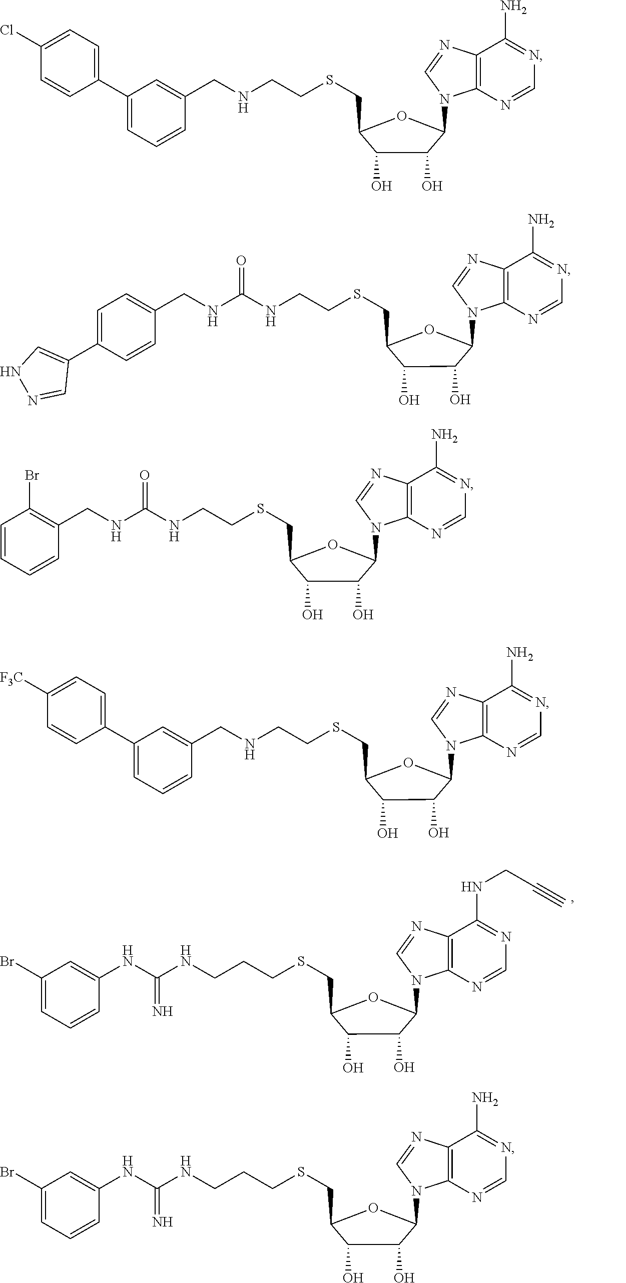

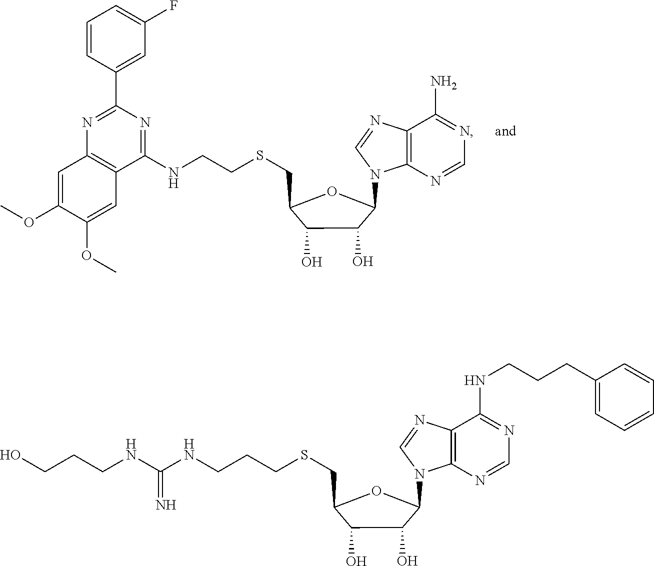 [SMILES: COc1cc2nc(-c3cccc(F)c3)nc(NCCSC[C@H]3O[C@@H](n4cnc5c(N)ncnc54)[C@H](O)[C@@H]3O)c2cc1OC ; "and" ; N=C(NCCCO)NCCCSC[C@H]1O[C@@H](n2cnc3c(NCCCc4ccccc4)ncnc32)[C@H](O)[C@@H]1O]

or a pharmaceutically acceptable salt thereof.

The present disclosure provides for a method for inhibiting PRMT activity comprising the step of administering to a mammal in need of relief from the effects of PRMT activity, a therapeutically effective amount of one or more compounds, wherein the compound is selected from the group of compounds listed in Table I, and having a formula (I), (II), (Ill), (IIa) and (IIIa).

The present disclosure provides for methods of using the compounds disclosed herein. Methods for the inhibition of PRMT. Methods of the specific inhibition of particular PRMT. Methods for modulated activity of targeted PRMT. Methods for treating diseases which are impacted by PRMT activity.

The present disclosure provides for pharmaceutical preparations comprising the compounds disclosed herein. Such pharmaceutical preparations may contain one or more such compounds. As disclosed herein, pharmaceutical preparations may contain one or more of the present compounds in combination with one or more other therapeutic agents and pharmaceutically acceptable salts thereof.

Generally, the present disclosure relates to compounds selected from the group consisting of the compounds listed in TABLE 1 below.

| | | | | | | PRMT6 | PRMT8 |
|---|---|---|---|---|---|---|---|
| Original | | | PRMT1 | PRMT3 | PRMT4 | (% | (% |
| ID | ID | Structure | (% inhibition | (% inhibition | (% inhibition | inhibition | inhibition |
| 1-205 | 5a | 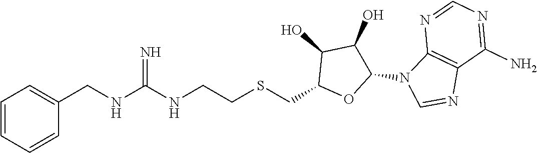 [SMILES: N=C(NCCSC[C@H]1O[C@@H](n2cnc3c(N)ncnc32)[C@H](O)[C@@H]1O)NCc1ccccc1] | 86 | 57 | 99 | 93 | 76 |
| | | NC1═NC═NC2═C1N═CN2[C@@H]3O[C@H](CSCCNC(NCC4═CC═ | | | | | |
| | | CC═C4)═N[C@@H](O)[C@H]3O | | | | | |
| 1-207 | 5b | 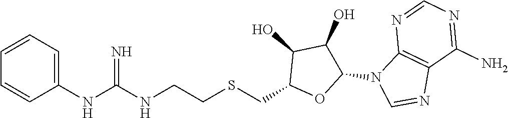 [SMILES: N=C(NCCSC[C@H]1O[C@@H](n2cnc3c(N)ncnc32)[C@H](O)[C@@H]1O)Nc1ccccc1] | 73 | 1 | 99 | 75 | 66 |
| | | NC1═NC═NC2═C1N═CN2[C@@H]3O[C@H](CSCCNC(NC4═CC═ | | | | | |
| | | CC═C4)═N)[C@@H](O)[C@H]3O | | | | | |
| 1-62 | 5c | 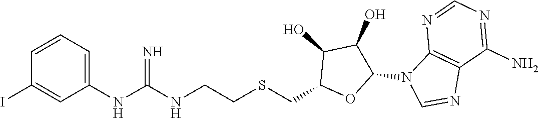 [SMILES: N=C(NCCSC[C@H]1O[C@@H](n2cnc3c(N)ncnc32)[C@H](O)[C@@H]1O)Nc1cccc(I)c1] | 95 | 38 | 100 | 98 | 99 |
| | | NC1═NC═NC2═C1N═CN2[C@@H]3O[C@H](CSCCNC(NC4═CC(I)═CC═ | | | | | |
| 1-208 | 5d | 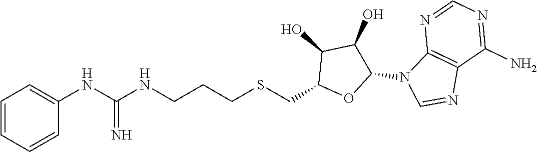 [SMILES: N=C(NCCCSC[C@H]1O[C@@H](n2cnc3c(N)ncnc32)[C@H](O)[C@@H]1O)Nc1ccccc1] | 94 | 54 | 99 | 99 | 98 |
| | | NC1═NC═NC2═C1N═CN2[C@@H]3O[C@H](CSCCCNC(NC4═CC═ | | | | | |
| | | CC═C4)═N)[C@@H](O)[C@H]3O | | | | | |
| 1-206 | 5e | 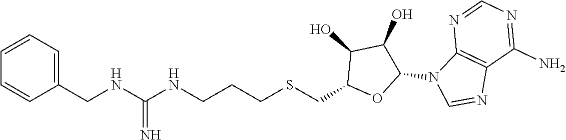 [SMILES: N=C(NCCCSC[C@H]1O[C@@H](n2cnc3c(N)ncnc32)[C@H](O)[C@@H]1O)NCc1ccccc1] | 95 | 61 | 97 | 98 | 97 |
| | | NC1═NC═NC2═C1N═CN2[C@@H]3O[C@H](CSCCCNC(NCC4═CC═ | | | | | |
| | | CC═C4)═N)[C@@H](O)[C@H]3O | | | | | |
| 1-59 | 5f | 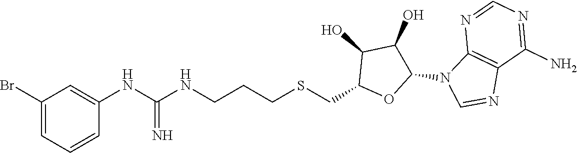 [SMILES: N=C(NCCCSC[C@H]1O[C@@H](n2cnc3c(N)ncnc32)[C@H](O)[C@@H]1O)Nc1cccc(Br)c1] | 94 | 68 | 100 | 100 | 100 |
| | | NC1═NC═NC2═C1N═CN2[C@@H]3O[C@H](CSCCCNC | | | | | |
| | | (NC4═CC(Br)═CC═C4)═N)[C@@H](O)[C@H]3O | | | | | |
| 1-198 | 6a | 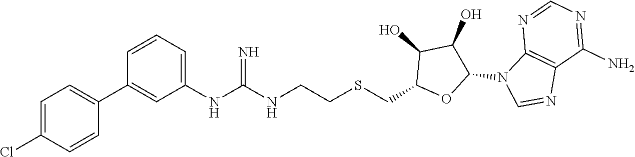 [SMILES: N=C(NCCSC[C@H]1O[C@@H](n2cnc3c(N)ncnc32)[C@H](O)[C@@H]1O)Nc1cccc(-c2ccc(Cl)cc2)c1] | 89 | 62 | 100 | 91 | 97 |
| | | NC1═NC═NC2═C1N═CN2[C@@H]3O[C@H](CSCCNC(NC4═ | | | | | |
| | | CC(C5═CC═C(Cl)C═C5)═CC═C4)═ | | | | | |
| | | N)[C@@H](O)[C@H]3O | | | | | |
| 1-200 | 6b | 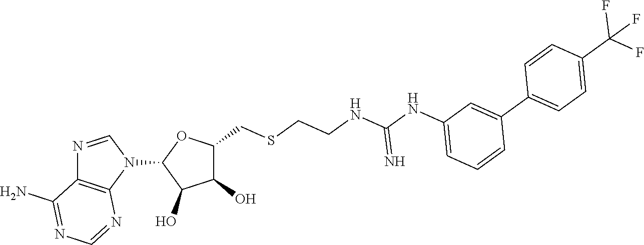 [SMILES: N=C(NCCSC[C@H]1O[C@@H](n2cnc3c(N)ncnc32)[C@H](O)[C@@H]1O)Nc1cccc(-c2ccc(C(F)(F)F)cc2)c1] | 83 | 42 | 100 | 91 | 94 |
| | | NC1═NC═NC2═C1N═CN2[C@@H]3O[C@H](CSCCNC(NC4═ | | | | | |
| | | CC(C5═CC═C(C(F)(F)F)C═C5)═CC═C4)═ | | | | | |
| | | N)[C@@H](O)[C@H]3O | | | | | |
| 1-201 | 6c | 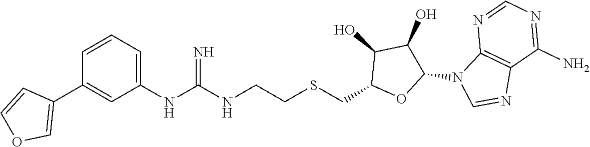 [SMILES: N=C(NCCSC[C@H]1O[C@@H](n2cnc3c(N)ncnc32)[C@H](O)[C@@H]1O)Nc1cccc(-c2ccoc2)c1] | 84 | 48 | 100 | 76 | 82 |
| | | NC1═NC═NC2═C1N═CN2[C@@H]3O[C@H](CSCCNC(NC4═CC(C5═COC═ | | | | | |
| | | C5)═CC═C4)═N)[C@@H](O)[C@H]3O | | | | | |
| 1-202 | 6d | 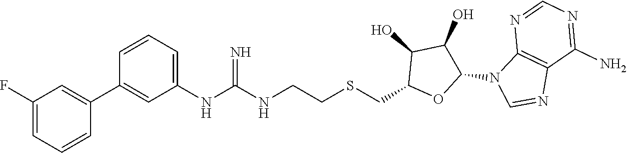 [SMILES: N=C(NCCSC[C@H]1O[C@@H](n2cnc3c(N)ncnc32)[C@H](O)[C@@H]1O)Nc1cccc(-c2cccc(F)c2)c1] | 68 | 26 | 100 | 67 | 81 |
| | | NC1═NC═NC2═C1N═CN2[C@@H]3O[C@H](CSCCNC(NC4═ | | | | | |
| | | CC(C5═CC(F)═CC═C5)═CC═C4)═ | | | | | |
| | | N)[C@@H](O)[C@H]3O | | | | | |
| 1-65 | 6e | 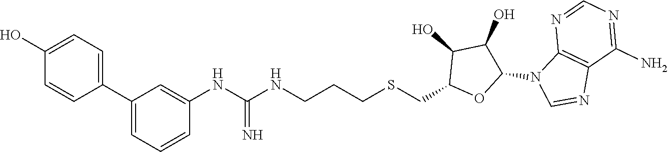 [SMILES: N=C(NCCCSC[C@H]1O[C@@H](n2cnc3c(N)ncnc32)[C@H](O)[C@@H]1O)Nc1cccc(-c2ccc(O)cc2)c1] | 95 | 47 | 100 | 99 | 98 |
| | | NC1═NC═NC2═C1N═CN2[C@@H]3O[C@H](CSCCCNC(NC4═ | | | | | |
| | | CC(C5═CC═C(O)C═C5)═ | | | | | |
| | | CC═C4)═N)[C@@H](O)[C@H]3O | | | | | |
| 1-66 | 6f | 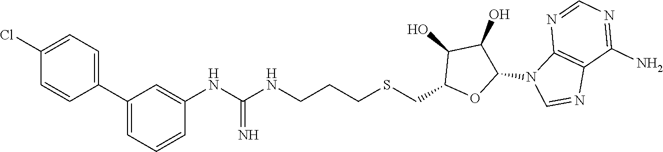 [SMILES: N=C(NCCCSC[C@H]1O[C@@H](n2cnc3c(N)ncnc32)[C@H](O)[C@@H]1O)Nc1cccc(-c2ccc(Cl)cc2)c1] | 92 | 76 | 100 | 97 | 100 |
| | | NC1═NC═NC2═C1N═CN2[C@@H]3O[C@H](CSCCCNC(NC4═ | | | | | |
| | | CC(C5═CC═C(O)C═C5)═CC═C4)═ | | | | | |
| | | N)[C@@H](O)[C@H]3O | | | | | |
| 1-68 | 6g | 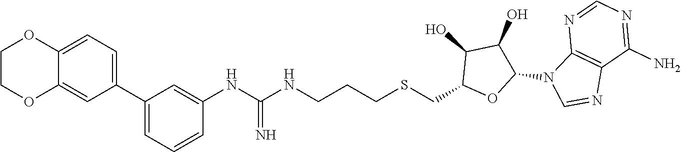 [SMILES: N=C(NCCCSC[C@H]1O[C@@H](n2cnc3c(N)ncnc32)[C@H](O)[C@@H]1O)Nc1cccc(-c2ccc3c(c2)OCCO3)c1] | 87 | 64 | 100 | 98 | 99 |
| | | NC1═NC═NC2═C1N═CN2[C@@H]3O[C@H](CSCCCNC(NC4═ | | | | | |
| | | CC(C5═CC(OCCO6)═ | | | | | |
| | | C6C═C5)═CC═C4)═N)[C@@H](O)[C@H]3O | | | | | |
| 1-69 | 6h | 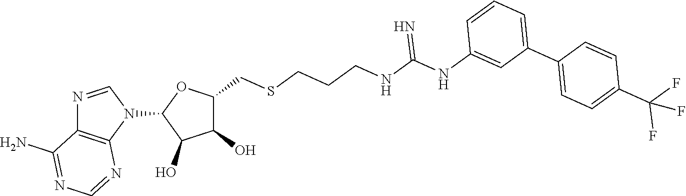 [SMILES: N=C(NCCCSC[C@H]1O[C@@H](n2cnc3c(N)ncnc32)[C@H](O)[C@@H]1O)Nc1cccc(-c2ccc(C(F)(F)F)cc2)c1] | 88 | 57 | 100 | 97 | 99 |
| | | NC1═NC═NC2═C1N═CN2[C@@H]3O[C@H](CSCCCNC(NC4═ | | | | | |
| | | CC(C5═CC═C(C(F)(F)F)C═ | | | | | |
| | | C5)═CC═C4)═N)[C@@H](O)[C@H]3O | | | | | |
| 1-70 | 6i | 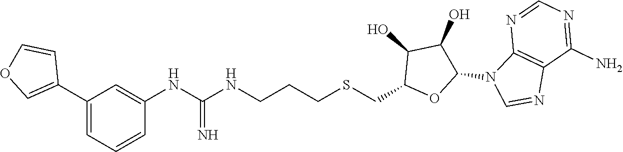 [SMILES: N=C(NCCCSC[C@H]1O[C@@H](n2cnc3c(N)ncnc32)[C@H](O)[C@@H]1O)Nc1cccc(-c2ccoc2)c1] | 95 | 46 | 100 | 95 | 98 |
| | | NC1═NC═NC2═C1N═CN2[C@@H]3O[C@H](CSCCCNC(NC4═ | | | | | |
| | | CC(C5═COC═C5)═CC═C4)═N)[C@@H](O)[C@H]3O | | | | | |
| 1-71 | 6j | 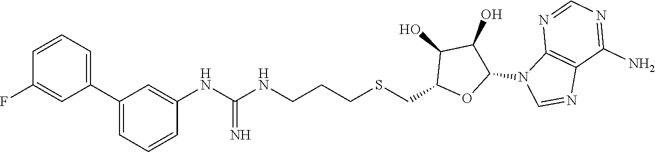 [SMILES: N=C(NCCCSC[C@H]1O[C@@H](n2cnc3c(N)ncnc32)[C@H](O)[C@@H]1O)Nc1cccc(-c2cccc(F)c2)c1] | 96 | 45 | 100 | 97 | 99 |
| | | NC1═NC═NC2═C1N═CN2[C@@H]3O[C@H](CSCCCNC(NC4═ | | | | | |
| | | CC(C5═CC(F)═CC═C5)═CC═C4)═N)[C@@H](O)[C@H]3O | | | | | |
| 1-72 | 6k | 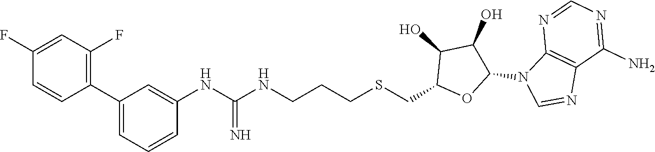 [SMILES: N=C(NCCCSC[C@H]1O[C@@H](n2cnc3c(N)ncnc32)[C@H](O)[C@@H]1O)Nc1cccc(-c2ccc(F)cc2F)c1] | 97 | 50 | 100 | 96 | 99 |
| | | NC1═NC═NC2═C1N═CN2[C@@H]3O[C@H](CSCCCNC(NC4═CC(C5═ | | | | | |
| | | C(F)C═C(F)C═C5)═CC═C4)═N)[C@@H](O)[C@H]3O | | | | | |
| 1-73 | 6l | 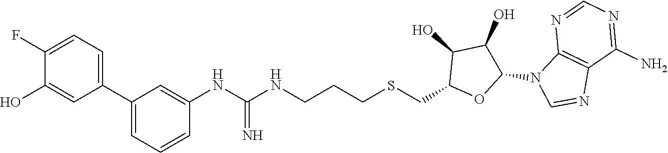 [SMILES: N=C(NCCCSC[C@H]1O[C@@H](n2cnc3c(N)ncnc32)[C@H](O)[C@@H]1O)Nc1cccc(-c2ccc(F)c(O)c2)c1] | 92 | 58 | 100 | 97 | 99 |
| | | NC1═NC═NC2═C1N═CN2[C@@H]3O[C@H](CSCCCNC(NC4═CC(C5═ | | | | | |
| | | CC(O)═C(F)C═C5)═CC═C4)═N)[C@@H](O)[C@H]3O | | | | | |
| 1-74 | 6m | 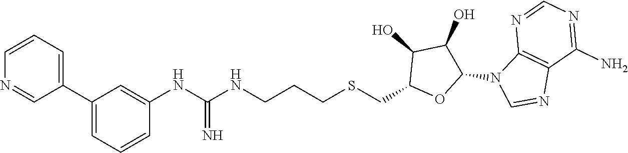 [SMILES: N=C(NCCCSC[C@H]1O[C@@H](n2cnc3c(N)ncnc32)[C@H](O)[C@@H]1O)Nc1cccc(-c2cccnc2)c1] | 88 | 22 | 100 | 93 | 96 |
| | | NC1═NC═NC2═C1N═CN2[C@@H]3O[C@H](CSCCCNC(NC4═CC(C5═ | | | | | |
| | | CN═CC═C5)═CC═C4)═N)[C@@H](O)[C@H]3O | | | | | |
| 1-75 | 6n | 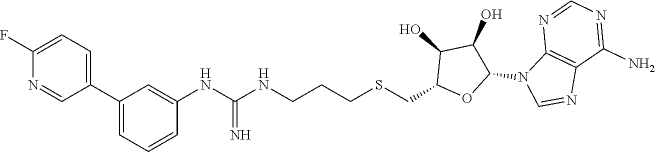 [SMILES: N=C(NCCCSC[C@H]1O[C@@H](n2cnc3c(N)ncnc32)[C@H](O)[C@@H]1O)Nc1cccc(-c2ccc(F)nc2)c1] | 91 | 61 | 100 | 99 | 100 |
| | | NC1═NC═NC2═C1N═CN2[C@@H]3O[C@H](CSCCCNC(NC4═ | | | | | |
| | | CC(C5═CN═CN═C5)═CC═C4)═N)[C@@H](O)[C@H]3O | | | | | |
| 1-76 | 6o | 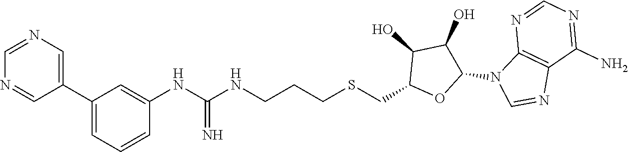 [SMILES: N=C(NCCCSC[C@H]1O[C@@H](n2cnc3c(N)ncnc32)[C@H](O)[C@@H]1O)Nc1cccc(-c2cncnc2)c1] | 102 | 5 | 100 | 96 | 95 |
| | | NC1═NC═NC2═C1N═CN2[C@@H]3O[C@H](CSCCCNC(NC4═CC(C5═ | | | | | |
| | | CC═CC([N+]([O−])═O)═C5)═CC═C4)═N)[C@@H](O)[C@H]3O | | | | | |
| 1-77 | 6p | 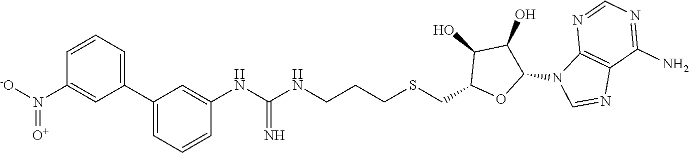 | 93 | 0 | 100 | 98 | 100 |
| | | NC1═NC═NC2═C1N═CN2[C@@H]3O[C@H](CSCCCNC(NC4═CC(C5═CC═ | | | | | |
| | | CC([N+]([O−])═O)═C5)═CC═C4)═N)[C@@H](O)[C@H]3O | | | | | |
| 1-79 | 6q | 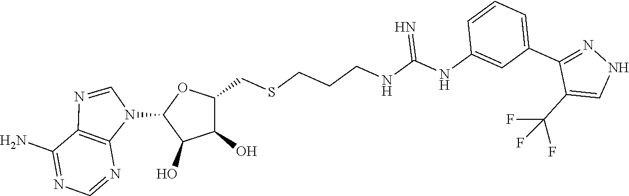 [SMILES: N=C(NCCCSC[C@H]1O[C@@H](n2cnc3c(N)ncnc32)[C@H](O)[C@@H]1O)Nc1cccc(-c2n[nH]cc2C(F)(F)F)c1] | 99 | 60 | 100 | 98 | 100 |
| | | NC1═NC═NC2═C1N═CN2[C@@H]3O[C@H](CSCCCNC(NC4═CC(C5═ | | | | | |
| | | NNC═C5C(F)(F)F)═CC═C4)═N)[C@@H](O)[C@H]3O | | | | | |
| 1-113 | 7a | 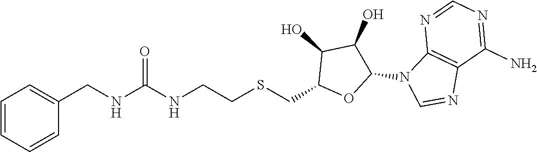 [SMILES: Nc1ncnc2c1ncn2[C@@H]1O[C@H](CSCCNC(=O)NCc2ccccc2)[C@@H](O)[C@H]1O] | 30 | 80 | 97 | 89 | 12 |
| | | NC1═NC═NC2═C1N═CN2[C@@H]3O[C@H](CSCCNC(NCC4═ | | | | | |
| | | CC═CC═C4)═O)[C@@H](O)[C@H]3O | | | | | |
| 1-114 | 7b | 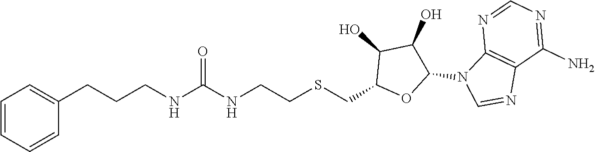 [SMILES: Nc1ncnc2c1ncn2[C@@H]1O[C@H](CSCCNC(=O)NCCCc2ccccc2)[C@@H](O)[C@H]1O] | 13 | 5 | 49 | 7 | −4 |
| | | NC1═NC═NC2═C1N═CN2[C@@H]3O[C@H](CSCCNC(NCCCC4═ | | | | | |
| | | CC═CC═C4)═O)[C@@H](O)[C@H]3O | | | | | |
| 1-188 | 7c | 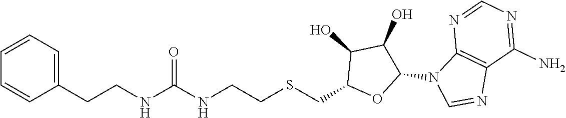 [SMILES: Nc1ncnc2c1ncn2[C@@H]1O[C@H](CSCCNC(=O)NCCc2ccccc2)[C@@H](O)[C@H]1O] | 8 | 6 | 53 | 22 | −1 |
| | | NC1═NC═NC2═C1N═CN2[C@@H]3O[C@H](CSCCNC(NCCC4═ | | | | | |
| | | CC═CC═C4)═O][C@@H](O)[C@H]3O | | | | | |
| 1-189 | 7d | 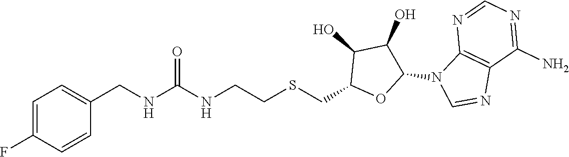 [SMILES: Nc1ncnc2c1ncn2[C@@H]1O[C@H](CSCCNC(=O)NCc2ccc(F)cc2)[C@@H](O)[C@H]1O] | 6 | 62 | 95 | 25 | 8 |
| | | NC1═NC═NC2═C1N═CN2[C@@H]3O[C@H](CSCCNC(NCC4═CC═ | | | | | |
| | | C(F)C═C4)═O)[C@@H](O)[C@H]3O | | | | | |
| 1-213 | 7e | 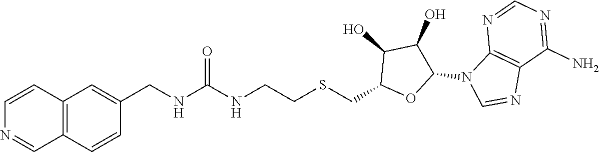 [SMILES: Nc1ncnc2c1ncn2[C@@H]1O[C@H](CSCCNC(=O)NCc2ccc3cnccc3c2)[C@@H](O)[C@H]1O] | 6 | 8 | 30 | 9 | −2 |
| | | NC1═NC═NC2═C1N═CN2[C@@H]3O[C@H](CSCCNC(NCC4═CC(C═ | | | | | |
| | | CN═C5)═C5C═C4)═O)[C@@H](O)[C@H]3O | | | | | |
| 1-214 | 7f | 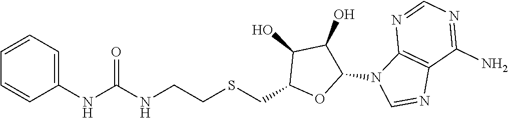 [SMILES: Nc1ncnc2c1ncn2[C@@H]1O[C@H](CSCCNC(=O)Nc2ccccc2)[C@@H](O)[C@H]1O] | 9 | 86 | 84 | 28 | 12 |
| | | NC1═NC═NC2═C1N═CN2[C@@H]3O[C@H](CSCCNC(NC4═ | | | | | |
| | | CC═CC═C4)═O)[C@@H](O)[C@H]3O | | | | | |
| 1-215 | 7g | 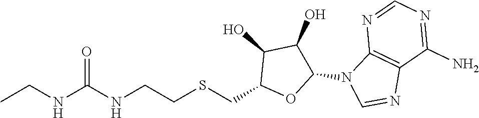 [SMILES: CCNC(=O)NCCSC[C@H]1O[C@@H](n2cnc3c(N)ncnc32)[C@H](O)[C@@H]1O] | 10 | 4 | 27 | 3 | 14 |
| | | NC1═NC═NC2═C1N═CN2[C@@H]3O[C@H](CSCCNC(NCC)═ | | | | | |
| | | O)[C@@H](O)[C@H]3O | | | | | |
| 1-234 | 7h | 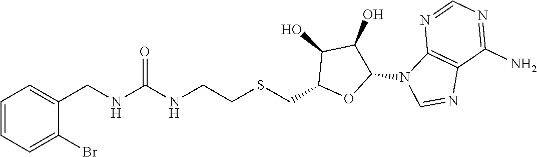 [SMILES: Nc1ncnc2c1ncn2[C@@H]1O[C@H](CSCCNC(=O)NCc2ccccc2Br)[C@@H](O)[C@H]1O] | 12 | 25 | 96 | −11 | −13 |
| | | NC1═NC═NC2═C1N═CN2[C@@H]3O[C@H](CSCCNC(NCC4═C(Br)C═ | | | | | |
| | | CC═C4)═O)[C@@H](O)[C@H]3O | | | | | |
| 1-235 | 7i | 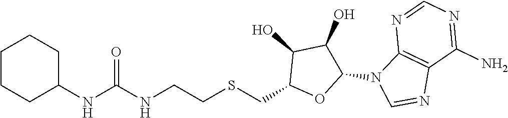 [SMILES: Nc1ncnc2c1ncn2[C@@H]1O[C@H](CSCCNC(=O)NC2CCCCC2)[C@@H](O)[C@H]1O] | 6 | 2 | 34 | −12 | −10 |
| | | NC1═NC═NC2═C1N═CN2[C@@H]3O[C@H](CSCCNC(NC4CCCCC4)═ | | | | | |
| | | O)[C@@H](O)[C@H]3O | | | | | |
| 1-236 | 7j | 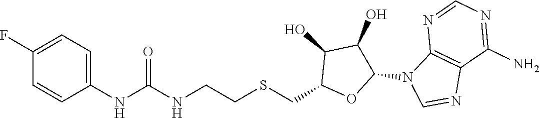 [SMILES: Nc1ncnc2c1ncn2[C@@H]1O[C@H](CSCCNC(=O)Nc2ccc(F)cc2)[C@@H](O)[C@H]1O] | 17 | 78 | 88 | 17 | 1 |
| | | NC1═NC═NC2═C1N═CN2[C@@H]3O[C@H](CSCCNC(NC4═CC═C(F)C═ | | | | | |
| | | C4)═O)[C@@H](O)[C@H]3O | | | | | |
| 1-237 | 7k | 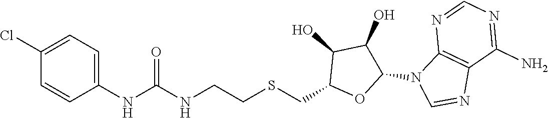 [SMILES: Nc1ncnc2c1ncn2[C@@H]1O[C@H](CSCCNC(=O)Nc2ccc(Cl)cc2)[C@@H](O)[C@H]1O] | 7 | 30 | 67 | 2 | −1 |
| | | NC1═NC═NC2═C1N═CN2[C@@H]3O[C@H](CSCCNC(NC4═CC═ | | | | | |
| | | C(Cl)C═C4)═O)[C@@H](O)[C@H]3O | | | | | |
| 1-238 | 7l | 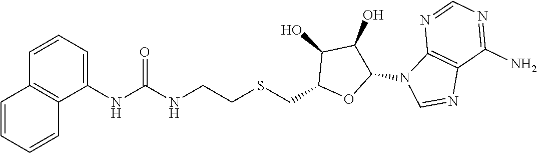 [SMILES: Nc1ncnc2c1ncn2[C@@H]1O[C@H](CSCCNC(=O)Nc2cccc3ccccc23)[C@@H](O)[C@H]1O] | 13 | 1 | 88 | 2 | 5 |
| | | NC1═NC═NC2═C1N═CN2[C@@H]3O[C@H](CSCCNC(NC4═C5C(C═CC═C5)═CC═ | | | | | |
| | | C4)═O)[C@@H](O)[C@H]3O | | | | | |
| 1-240 | 7m | 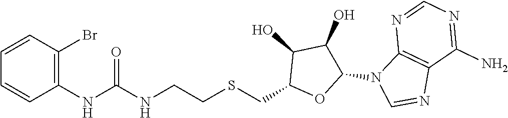 [SMILES: Nc1ncnc2c1ncn2[C@@H]1O[C@H](CSCCNC(=O)Nc2ccccc2Br)[C@@H](O)[C@H]1O] | 10 | 55 | 81 | 11 | 13 |
| | | NC1═NC═NC2═C1N═CN2[C@@H]3O[C@H](CSCCNC(NC4═ | | | | | |
| | | C(Br)C═CC═C4)═O)[C@@H](O)[C@H]3O | | | | | |
| 1-117 | 7o | 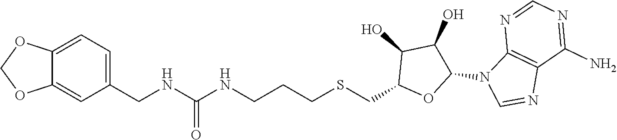 [SMILES: Nc1ncnc2c1ncn2[C@@H]1O[C@H](CSCCCNC(=O)NCc2ccc3c(c2)OCO3)[C@@H](O)[C@H]1O] | 5 | 5 | 42 | 89 | 7 |
| | | NC1═NC═NC2═C1N═CN2[C@@H]3O[C@H](CSCCCNC(NCC4═CC═C(OCO5)C5═ | | | | | |
| | | C4)═O)[C@@H](O)[C@H]3O | | | | | |
| 1-191 | 7p | 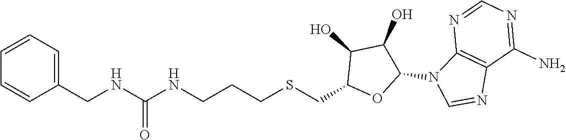 [SMILES: Nc1ncnc2c1ncn2[C@@H]1O[C@H](CSCCCNC(=O)NCc2ccccc2)[C@@H](O)[C@H]1O] | 7 | 13 | 76 | 67 | 12 |
| | | NC1═NC═NC2═C1N═CN2[C@@H]3O[C@H](CSCCCNC(NCC4═CC═CC═ | | | | | |
| | | C4)═O)[C@@H](O)[C@H]3O | | | | | |
| 1-192 | 7q | 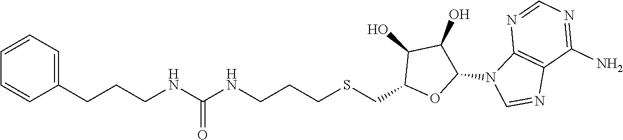 [SMILES: Nc1ncnc2c1ncn2[C@@H]1O[C@H](CSCCCNC(=O)NCCCc2ccccc2)[C@@H](O)[C@H]1O] | 18 | 5 | 62 | −6 | 16 |
| | | NC1═NC═NC2═C1N═CN2[C@@H]3O[C@H](CSCCCNC(NCCCC4═CC═ | | | | | |
| | | CC═C4)═O)[C@@H](O)[C@H]3O | | | | | |
| 1-194 | 7r | 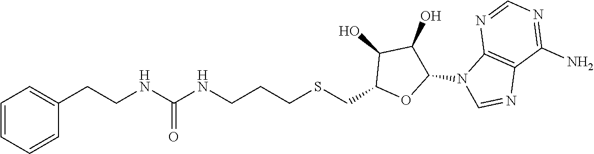 [SMILES: Nc1ncnc2c1ncn2[C@@H]1O[C@H](CSCCCNC(=O)NCCc2ccccc2)[C@@H](O)[C@H]1O] | 14 | 6 | 47 | 49 | 22 |
| | | NC1═NC═NC2═C1N═CN2[C@@H]3O[C@H](CSCCCNC(NCCC4═CC═CC═ | | | | | |
| | | C4)═O][C@@H](O)[C@H]3O | | | | | |
| 1-195 | 7s | 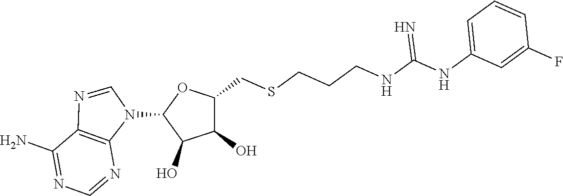 [SMILES: N=C(NCCCSC[C@H]1O[C@@H](n2cnc3c(N)ncnc32)[C@H](O)[C@@H]1O)Nc1cccc(F)c1] | 23 | 9 | 58 | 49 | −12 |
| | | NC1═NC═NC2═C1N═CN2[C@@H]3O[C@H](CSCCCNC(NCC4═CC═C(F)C═ | | | | | |
| | | C4)═O)[C@@H](O)[C@H]3O | | | | | |
| 1-225 | 8a | 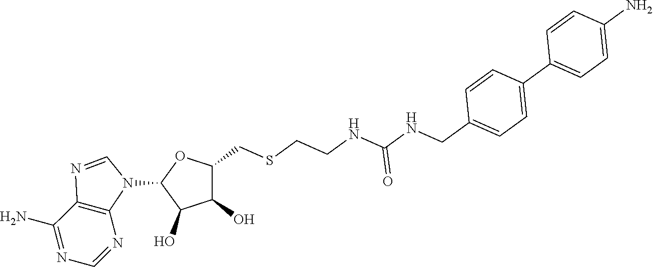 [SMILES: Nc1ccc(-c2ccc(CNC(=O)NCCSC[C@H]3O[C@@H](n4cnc5c(N)ncnc54)[C@H](O)[C@@H]3O)cc2)cc1] | 13 | 3 | 40 | 72 | 0 |
| | | NC1═NC═NC2═C1N═CN2[C@@H]3O[C@H](CSCCNC(NCC4═CC═C(C5═CC═ | | | | | |
| | | C(N)C═C5)C═C4)═O)[C@@H](O)[C@H]3O | | | | | |
| 1-226 | 8b | 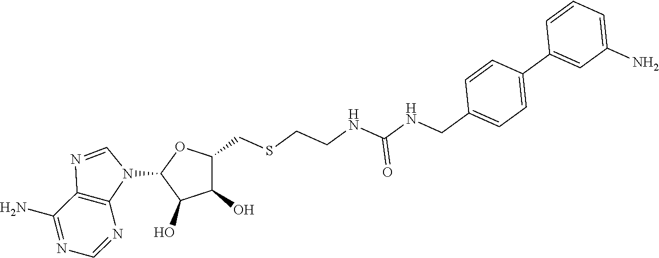 [SMILES: Nc1cccc(-c2ccc(CNC(=O)NCCSC[C@H]3O[C@@H](n4cnc5c(N)ncnc54)[C@H](O)[C@@H]3O)cc2)c1] | 7 | 5 | 43 | 79 | 0 |
| | | NC1═NC═NC2═C1N═CN2[C@@H]3O[C@H](CSCCNC(NCC4═CC═C(C5═CC(N)═ | | | | | |
| | | CC═C5)C═C4)═O)[C@@H](O)[C@H]3O | | | | | |
| 1-227 | 8c | 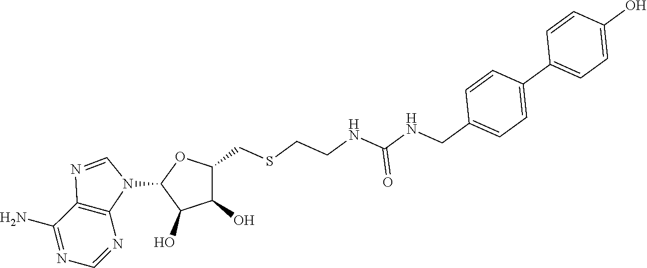 [SMILES: Nc1ncnc2c1ncn2[C@@H]1O[C@H](CSCCNC(=O)NCc2ccc(-c3ccc(O)cc3)cc2)[C@@H](O)[C@H]1O] | 21 | 10 | 42 | 82 | 3 |
| | | NC1═NC═NC2═C1N═CN2[C@@H]3O[C@H](CSCCNC(NCC4═CC═ | | | | | |
| | | C(C5═CC═C(O)C═C5)C═C4)═O)[C@@H](O)[C@H]3O | | | | | |
| 1-228 | 8d | 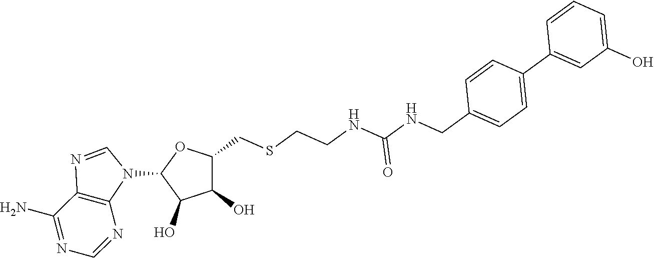 [SMILES: Nc1ncnc2c1ncn2[C@@H]1O[C@H](CSCCNC(=O)NCc2ccc(-c3cccc(O)c3)cc2)[C@@H](O)[C@H]1O] | 22 | 9 | 37 | 88 | 15 |
| | | NC1═NC═NC2═C1N═CN2[C@@H]3O[C@H](CSCCNC(NCC4═CC═C(C5═ | | | | | |
| | | CC(O)═CC═C5)C═C4)═O)[C@@H](O)[C@H]3O | | | | | |
| 1-229 | 8e | 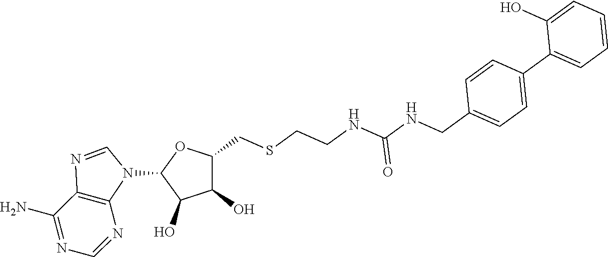 [SMILES: Nc1ncnc2c1ncn2[C@@H]1O[C@H](CSCCNC(=O)NCc2ccc(-c3ccccc3O)cc2)[C@@H](O)[C@H]1O] | 11 | 13 | 53 | −2 | −2 |
| | | NC1═NC═NC2═C1N═CN2[C@@H]3O[C@H](CSCCNC(NCC4═CC═C(C5═ | | | | | |
| | | C(O)C═CC═C5)C═C4)═O)[C@@H](O)[C@H]3O | | | | | |
| 1-230 | 8f | 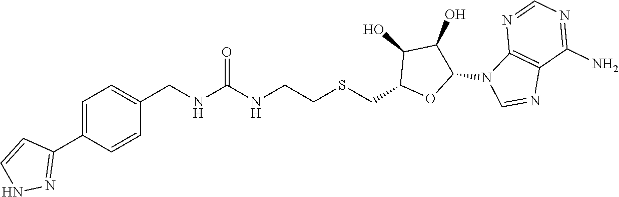 [SMILES: Nc1ncnc2c1ncn2[C@@H]1O[C@H](CSCCNC(=O)NCc2ccc(-c3cc[nH]n3)cc2)[C@@H](O)[C@H]1O] | 27 | 9 | 17 | −6 | 2 |
| | | NC1═NC═NC2═C1N═CN2[C@@H]3O[C@H](CSCCNC(NCC4═CC═ | | | | | |
| | | C(C5═NNC═C5)C═C4)═O)[C@@H](O)[C@H]3O | | | | | |
| 1-231 | 8g | 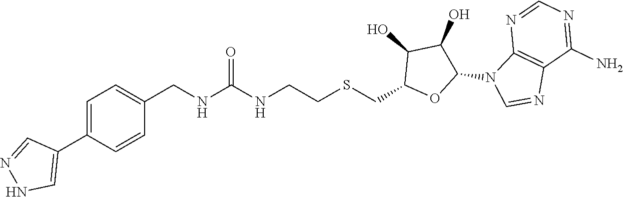 [SMILES: Nc1ncnc2c1ncn2[C@@H]1O[C@H](CSCCNC(=O)NCc2ccc(-c3cn[nH]c3)cc2)[C@@H](O)[C@H]1O] | 2 | 10 | 19 | 5 | 5 |
| | | NC1═NC═NC2═C1N═CN2[C@@H]3O[C@H](CSCCNC(NCC4═CC═ | | | | | |
| | | C(C5═CNN═C5)C═C4)═O)[C@@H](O)[C@H]3O | | | | | |
| 1-130 | 9a | 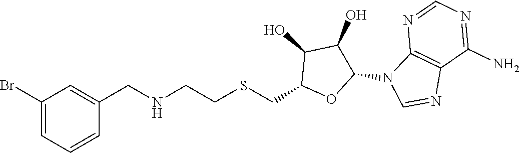 [SMILES: Nc1ncnc2c1ncn2[C@@H]1O[C@H](CSCCNCc2cccc(Br)c2)[C@@H](O)[C@H]1O] | 11 | 8 | 100 | 58 | 47 |
| | | NC1═NC═NC2═C1N═CN2[C@@H]3O[C@H](CSCCNCC4═CC(Br)═ | | | | | |
| | | CC═C4)[C@@H](O)[C@H]3O | | | | | |
| 1-131 | 10a | 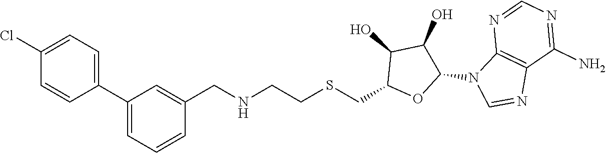 [SMILES: Nc1ncnc2c1ncn2[C@@H]1O[C@H](CSCCNCc2cccc(-c3ccc(Cl)cc3)c2)[C@@H](O)[C@H]1O] | 16 | 16 | 99 | 57 | 71 |
| | | NC1═NC═NC2═C1N═CN2[C@@H]3O[C@H](CSCCNCC4═CC(C5═CC═ | | | | | |
| | | C(Cl)C═C5)═CC═C4)[C@@H](O)[C@H]3O | | | | | |
| 1-132 | 10b | 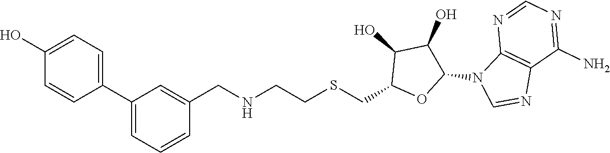 [SMILES: Nc1ncnc2c1ncn2[C@@H]1O[C@H](CSCCNCc2cccc(-c3ccc(O)cc3)c2)[C@@H](O)[C@H]1O] | 19 | 1 | 100 | 55 | 67 |
| | | NC1═NC═NC2═C1N═CN2[C@@H]3O[C@H](CSCCNCC4═CC(C5═CC═ | | | | | |
| | | C(O)C═C5)═CC═C4)[C@@H](O)[C@H]3O | | | | | |
| 1-133 | 10c | 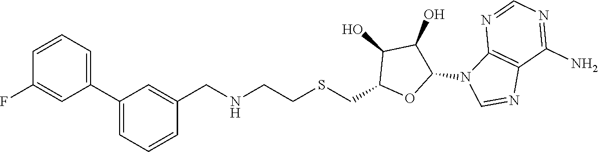 [SMILES: Nc1ncnc2c1ncn2[C@@H]1O[C@H](CSCCNCc2cccc(-c3cccc(F)c3)c2)[C@@H](O)[C@H]1O] | 11 | 12 | 100 | 49 | 68 |
| | | NC1═NC═NC2═C1N═CN2[C@@H]3O[C@H](CSCCNCC4═CC(C5═CC═ | | | | | |
| | | CC(F)═C5)═CC═C4)[C@@H](O)[C@H]3O | | | | | |
| 1-134 | 10d | 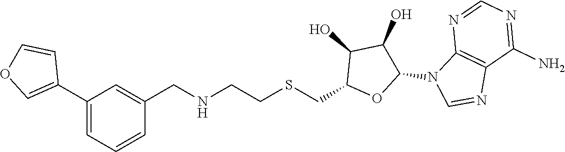 [SMILES: Nc1ncnc2c1ncn2[C@@H]1O[C@H](CSCCNCc2cccc(-c3ccoc3)c2)[C@@H](O)[C@H]1O] | 14 | 1 | 100 | 46 | 44 |
| | | NC1═NC═NC2═C1N═CN2[C@@H]3O[C@H](CSCCNCC4═CC(C5═ | | | | | |
| | | COC═C5)═CC═C4)[C@@H](O)[C@H]3O | | | | | |
| 1-135 | 10e | 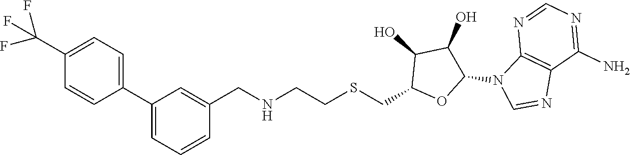 [SMILES: Nc1ncnc2c1ncn2[C@@H]1O[C@H](CSCCNCc2cccc(-c3ccc(C(F)(F)F)cc3)c2)[C@@H](O)[C@H]1O] | 2 | 15 | 99 | 57 | 67 |
| | | NC1═NC═NC2═C1N═CN2[C@@H]3O[C@H](CSCCNCC4═CC(C5═ | | | | | |
| | | CC═C(C(F)(F)F)C═C5)═CC═C4)[C@@H](O)[C@H]3O | | | | | |
| 1-136 | 10f | 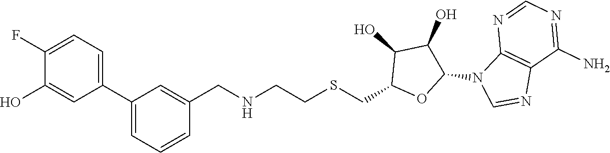 [SMILES: Nc1ncnc2c1ncn2[C@@H]1O[C@H](CSCCNCc2cccc(-c3ccc(F)c(O)c3)c2)[C@@H](O)[C@H]1O] | 7 | 20 | 99 | 54 | 80 |
| | | NC1═NC═NC2═C1N═CN2[C@@H]3O[C@H](CSCCNCC4═CC(C5═ | | | | | |
| | | CC═C(F)C(O)═C5)═CC═C4)[C@@H](O)[C@H]3O | | | | | |
| 1-138 | 10g | 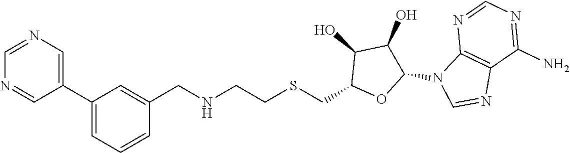 [SMILES: Nc1ncnc2c1ncn2[C@@H]1O[C@H](CSCCNCc2cccc(-c3cncnc3)c2)[C@@H](O)[C@H]1O] | 5 | 13 | 98 | 31 | 36 |
| | | NC1═NC═NC2═C1N═CN2[C@@H]3O[C@H](CSCCNCC4═ | | | | | |
| | | CC(C5═CN═CN═C5)═CC═C4)[C@@H](O)[C@H]3O | | | | | |
| 1-139 | 10h | 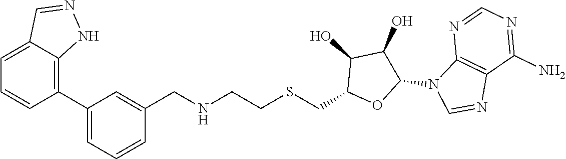 [SMILES: Nc1ncnc2c1ncn2[C@@H]1O[C@H](CSCCNCc2cccc(-c3cccc4cn[nH]c34)c2)[C@@H](O)[C@H]1O] | 1 | 12 | 100 | 82 | 58 |
| | | NC1═NC═NC2═C1N═CN2[C@@H]3O[C@H](CSCCNCC4═CC(C5═ | | | | | |
| | | CC═CC6═C5NN═C6)═CC═C4)[C@@H](O)[C@H]3O | | | | | |
| 1-140 | 10i | 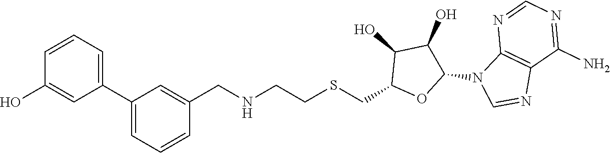 [SMILES: Nc1ncnc2c1ncn2[C@@H]1O[C@H](CSCCNCc2cccc(-c3cccc(O)c3)c2)[C@@H](O)[C@H]1O] | 15 | 13 | 100 | 55 | 74 |
| | | NC1═NC═NC2═C1N═CN2[C@@H]3O[C@H](CSCCNCC4═ | | | | | |
| | | CC(C5═CC═CC(O)═C5)═CC═C4)[C@@H](O)[C@H]3O | | | | | |
| 1-141 | 10j | 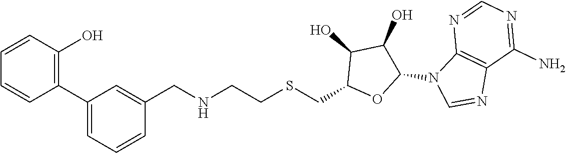 [SMILES: Nc1ncnc2c1ncn2[C@@H]1O[C@H](CSCCNCc2cccc(-c3ccccc3O)c2)[C@@H](O)[C@H]1O] | 25 | 10 | 99 | 43 | 56 |
| | | NC1═NC═NC2═C1N═CN2[C@@H]3O[C@H](CSCCNCC4═ | | | | | |
| | | CC(C5═CC═CC═C5O)═CC═C4)[C@@H](O)[C@H]3O | | | | | |
| 1-142 | 10k | 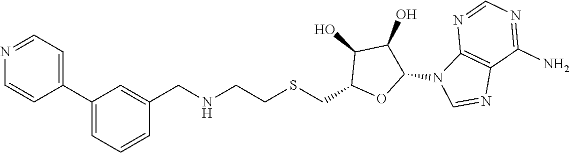 [SMILES: Nc1ncnc2c1ncn2[C@@H]1O[C@H](CSCCNCc2cccc(-c3ccncc3)c2)[C@@H](O)[C@H]1O] | 15 | 10 | 99 | 43 | 69 |
| | | NC1═NC═NC2═C1N═CN2[C@@H]3O[C@H](CSCCNCC4═CC(C5═ | | | | | |
| | | CC═NC═C5)═CC═C4)[C@@H](O)[C@H]3O | | | | | |
| 1-144 | 10l | 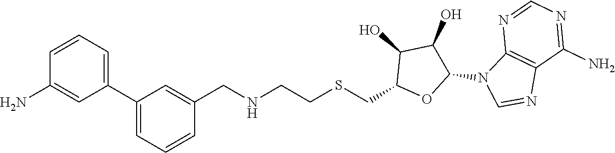 [SMILES: Nc1cccc(-c2cccc(CNCCSC[C@H]3O[C@@H](n4cnc5c(N)ncnc54)[C@H](O)[C@@H]3O)c2)c1] | 7 | 8 | 100 | 53 | 62 |
| | | NC1═NC═NC2═C1N═CN2[C@@H]3O[C@H](CSCCNCC4═CC(C5═ | | | | | |
| | | CC═CC(N)═C5)═CC═C4)[C@@H](O)[C@H]3O | | | | | |
| 1-101 | 10m | 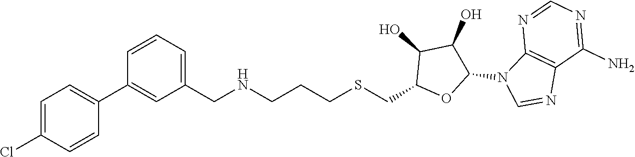 [SMILES: Nc1ncnc2c1ncn2[C@@H]1O[C@H](CSCCCNCc2cccc(-c3ccc(Cl)cc3)c2)[C@@H](O)[C@H]1O] | 65 | 22 | 99 | 46 | 73 |
| | | NC1═NC═NC2═C1N═CN2[C@@H]3O[C@H](CSCCCNCC4═ | | | | | |
| | | CC═CC(C5═CC═C(Cl)C═C5)═C4)[C@@H](O)[C@H]3O | | | | | |
| 1-102 | 10n | 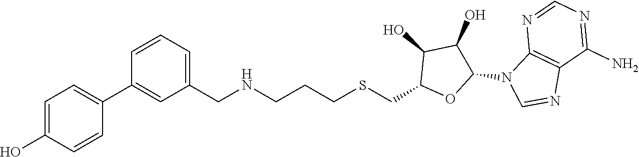 [SMILES: Nc1ncnc2c1ncn2[C@@H]1O[C@H](CSCCCNCc2cccc(-c3ccc(O)cc3)c2)[C@@H](O)[C@H]1O] | 57 | 21 | 96 | 36 | 69 |
| | | NC1═NC═NC2═C1N═CN2[C@@H]3O[C@H](CSCCCNCC4═ | | | | | |
| | | CC═CC(C5═CC═C(O)C═C5)═C4)[C@@H](O)[C@H]3O | | | | | |
| 1-103 | 10o | 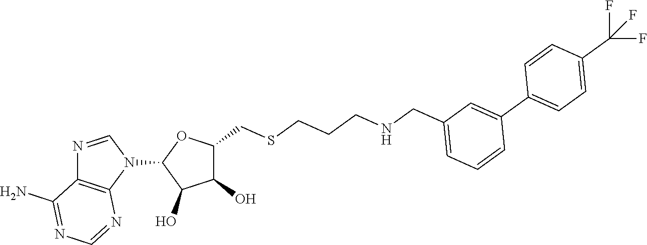 [SMILES: Nc1ncnc2c1ncn2[C@@H]1O[C@H](CSCCCNCc2cccc(-c3ccc(C(F)(F)F)cc3)c2)[C@@H](O)[C@H]1O] | 71 | 24 | 97 | 37 | 65 |
| | | NC1═NC═NC2═C1N═CN2[C@@H]3O[C@H](CSCCCNCC4═CC═CC(C5═ | | | | | |
| | | CC═C(C(F)(F)F)C═C5)═C4)[C@@H](O)[C@H]3O | | | | | |
| 1-104 | 10p | 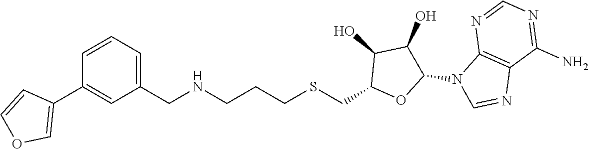 [SMILES: Nc1ncnc2c1ncn2[C@@H]1O[C@H](CSCCCNCc2cccc(-c3ccoc3)c2)[C@@H](O)[C@H]1O] | 54 | 25 | 99 | 66 | 54 |
| | | NC1═NC═NC2═C1N═CN2[C@@H]3O[C@H](CSCCCNCC4═ | | | | | |
| | | CC═CC(C5═COC═C5)═C4)[C@@H](O)[C@H]3O | | | | | |
| 1-105 | 10q | 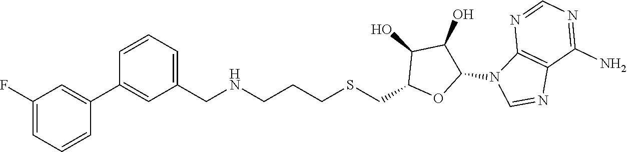 [SMILES: Nc1ncnc2c1ncn2[C@@H]1O[C@H](CSCCCNCc2cccc(-c3cccc(F)c3)c2)[C@@H](O)[C@H]1O] | 63 | 25 | 97 | 36 | 66 |
| | | NC1═NC═NC2═C1N═CN2[C@@H]3O[C@H](CSCCCNCC4═ | | | | | |
| | | CC═CC(C5═CC(F)═CC═C5)═C4)[C@@H](O)[C@H]3O | | | | | |
| 1-106 | 10r | 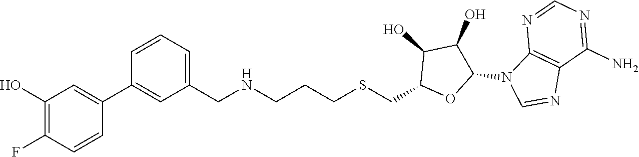 [SMILES: Nc1ncnc2c1ncn2[C@@H]1O[C@H](CSCCCNCc2cccc(-c3ccc(F)c(O)c3)c2)[C@@H](O)[C@H]1O] | 52 | 22 | 95 | 74 | 72 |
| | | NC1═NC═NC2═C1N═CN2[C@@H]3O[C@H](CSCCCNCC4═ | | | | | |
| | | CC═CC(C5═CC(O)═C(F)C═C5)═C4)[C@@H](O)[C@H]3O | | | | | |
| 1-108 | 10s | 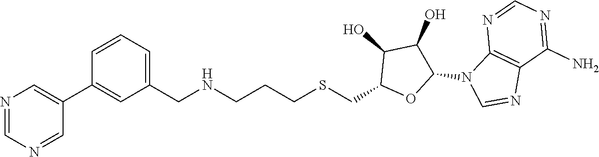 [SMILES: Nc1ncnc2c1ncn2[C@@H]1O[C@H](CSCCCNCc2cccc(-c3cncnc3)c2)[C@@H](O)[C@H]1O] | 28 | 24 | 97 | 65 | 39 |
| | | NC1═NC═NC2═C1N═CN2[C@@H]3O[C@H](CSCCCNCC4═CC═ | | | | | |
| | | CC(C5═CN═CN═C5)═C4)[C@@H](O)[C@H]3O | | | | | |
| 1-109 | 10t | 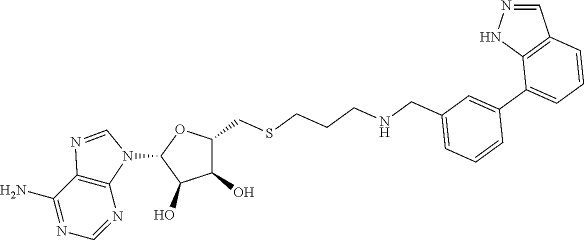 [SMILES: Nc1ncnc2c1ncn2[C@@H]1O[C@H](CSCCCNCc2cccc(-c3cccc4cn[nH]c34)c2)[C@@H](O)[C@H]1O] | 69 | 22 | 99 | 87 | 72 |
| | | NC1═NC═NC2═C1N═CN2[C@@H]3O[C@H](CSCCCNCC4═ | | | | | |
| | | CC═CC(C5═C(NN═C6)C6═CC═C5)═C4)[C@@H](O)[C@H]3O | | | | | |
| 1-145 | 12a | 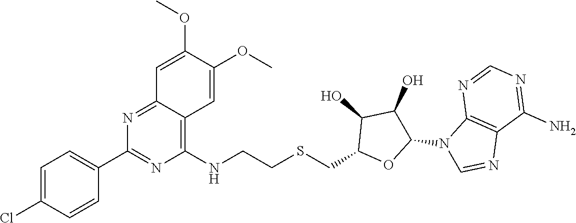 [SMILES: COc1cc2nc(-c3ccc(Cl)cc3)nc(NCCSC[C@H]3O[C@@H](n4cnc5c(N)ncnc54)[C@H](O)[C@@H]3O)c2cc1OC] | 1 | 4 | 69 | 85 | 23 |
| | | NC1═NC═NC2═C1N═CN2[C@@H]3O[C@H](CSCCNC4═NC(C5═CC═ | | | | | |
| | | C(Cl)C═C5)═NC6═C4C═C(OC)C(OC)═C6)[C@@H](O)[C@H]3O | | | | | |
| 1-146 | 12b | 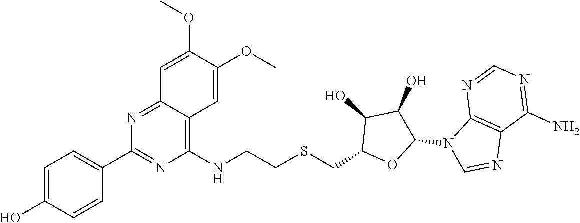 [SMILES: COc1cc2nc(-c3ccc(O)cc3)nc(NCCSC[C@H]3O[C@@H](n4cnc5c(N)ncnc54)[C@H](O)[C@@H]3O)c2cc1OC] | 2 | 13 | −4 | 91 | 22 |
| | | NC1═NC═NC2═C1N═CN2[C@@H]3O[C@H](CSCCNC4═NC(C5═CC═ | | | | | |
| | | C(O)C═C5)═NC6═C4C═C(OC)C(OC)═C6)[C@@H](O)[C@H]3O | | | | | |
| 1-147 | 12c | 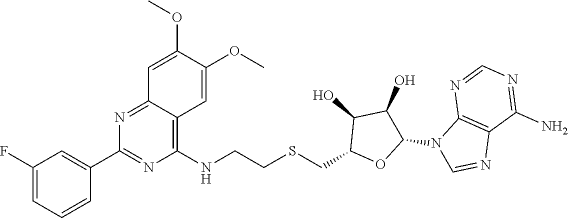 [SMILES: COc1cc2nc(-c3cccc(F)c3)nc(NCCSC[C@H]3O[C@@H](n4cnc5c(N)ncnc54)[C@H](O)[C@@H]3O)c2cc1OC] | 15 | 15 | 62 | 43 | 35 |
| | | NC1═NC═NC2═C1N═CN2[C@@H]3O[C@H](CSCCNC4═NC(C5═ | | | | | |
| | | CC(F)═CC═C5)═NC6═C4C═C(OC)C(OC)═C6)[C@@H](O)[C@H]3O | | | | | |
| 1-148 | 12d | 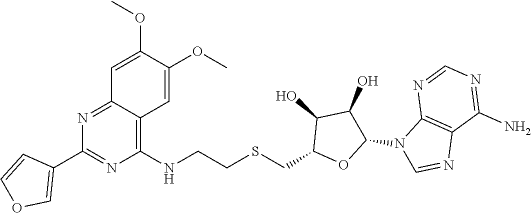 [SMILES: COc1cc2nc(-c3ccoc3)nc(NCCSC[C@H]3O[C@@H](n4cnc5c(N)ncnc54)[C@H](O)[C@@H]3O)c2cc1OC] | 2 | 10 | 1 | 16 | 25 |
| | | NC1═NC═NC2═C1N═CN2[C@@H]3O[C@H](CSCCNC4═NC(C5═ | | | | | |
| | | COC═C5)═NC6═C4C═C(OC)C(OC)═C6)[C@@H](O)[C@H]3O | | | | | |
| 1-149 | 12e | 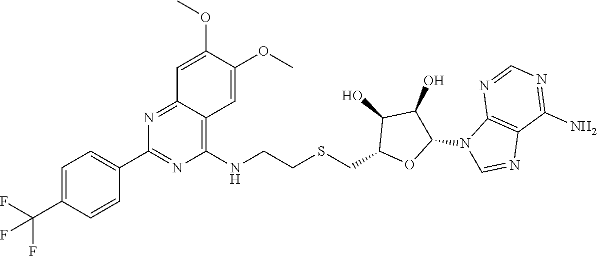 [SMILES: COc1cc2nc(-c3ccc(C(F)(F)F)cc3)nc(NCCSC[C@H]3O[C@@H](n4cnc5c(N)ncnc54)[C@H](O)[C@@H]3O)c2cc1OC] | 3 | 5 | 43 | 19 | 37 |
| | | NC1═NC═NC2═C1N═CN2[C@@H]3O[C@H](CSCCNC4═NC(C5═CC═ | | | | | |
| | | C(C(F)(F)F)C═C5)═NC6═C4C═C(OC)C(OC)═C6)[C@@H](O)[C@H]3O | | | | | |
| 1-150 | 12f | 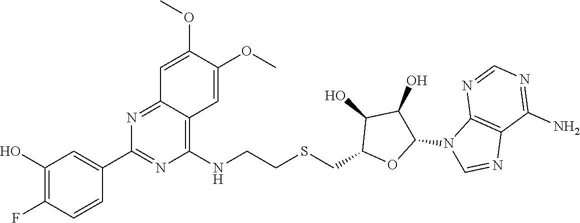 [SMILES: COc1cc2nc(-c3ccc(F)c(O)c3)nc(NCCSC[C@H]3O[C@@H](n4cnc5c(N)ncnc54)[C@H](O)[C@@H]3O)c2cc1OC] | 12 | 9 | 51 | 16 | 21 |
| | | NC1═NC═NC2═C1N═CN2[C@@H]3O[C@H](CSCCNC4═NC(C5═CC(O)═ | | | | | |
| | | C(F)C═C5)═NC6═C4C═C(OC)C(OC)═C6)[C@@H](O)[C@H]3O | | | | | |
| 1-151 | 12g | 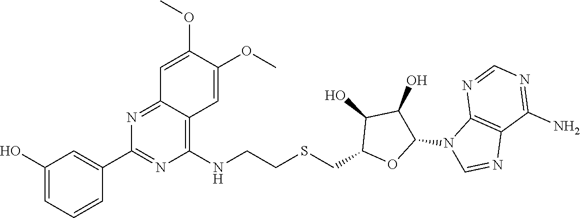 [SMILES: COc1cc2nc(-c3cccc(O)c3)nc(NCCSC[C@H]3O[C@@H](n4cnc5c(N)ncnc54)[C@H](O)[C@@H]3O)c2cc1OC] | 2 | 4 | 10 | 13 | 25 |
| | | NC1═NC═NC2═C1N═CN2[C@@H]3O[C@H](CSCCNC4═NC(C5═CC(O)═ | | | | | |
| | | CC═C5)═NC6═C4C═C(OC)C(OC)═C6)[C@@H](O)[C@H]3O | | | | | |
| 1-152 | 12h | 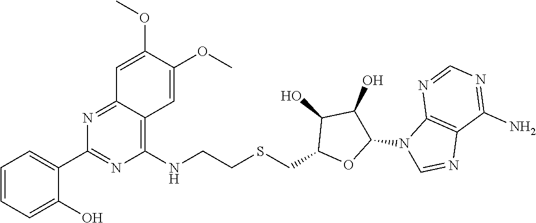 [SMILES: COc1cc2nc(-c3ccccc3O)nc(NCCSC[C@H]3O[C@@H](n4cnc5c(N)ncnc54)[C@H](O)[C@@H]3O)c2cc1OC] | 1 | 12 | 36 | 16 | 18 |
| | | NC1═NC═NC2═C1N═CN2[C@@H]3O[C@H](CSCCNC4═NC(C5═C(O)C═ | | | | | |
| | | CC═C5)═NC6═C4C═C(OC)C(OC)═C6)[C@@H](O)[C@H]3O | | | | | |
| 1-153 | 12i | 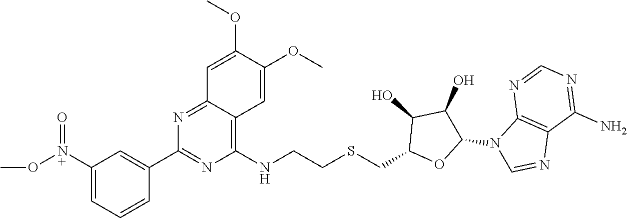 | 14 | 10 | 83 | 17 | 25 |
| | | NC1═NC═NC2═C1N═CN2[C@@H]3O[C@H](CSCCNC4═NC(C5═CC([N+]([O−])═ | | | | | |
| | | O)═CC═C5)═NC6═C4C═C(OC)C(OC)═C6)[C@@H](O)[C@H]3O | | | | | |
| 1-154 | 12j | 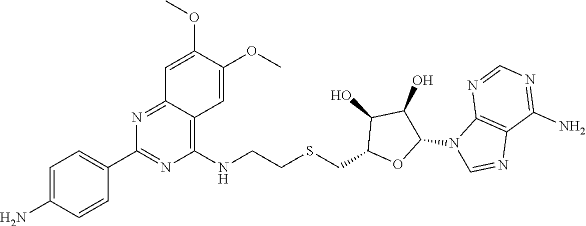 [SMILES: COc1cc2nc(-c3ccc(N)cc3)nc(NCCSC[C@H]3O[C@@H](n4cnc5c(N)ncnc54)[C@H](O)[C@@H]3O)c2cc1OC] | 12 | 13 | 1 | 8 | 21 |
| | | NC1═NC═NC2═C1N═CN2[C@@H]3O[C@H](CSCCNC4═NC(C5═CC═C(N)C═ | | | | | |
| | | C5)═NC6═C4C═C(OC)C(OC)═C6)[C@@H](O)[C@H]3O | | | | | |
| 1-157 | 14a | 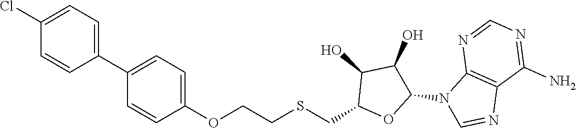 [SMILES: Nc1ncnc2c1ncn2[C@@H]1O[C@H](CSCCOc2ccc(-c3ccc(Cl)cc3)cc2)[C@@H](O)[C@H]1O] | 2 | 9 | 47 | −4 | 38 |
| | | NC1═NC═NC2═C1N═CN2[C@@H]3O[C@H](CSCCOC4═CC═C(C5═ | | | | | |
| | | CC═C(Cl)C═C5)C═C4)[C@@H](O)[C@H]3O | | | | | |
| 1-158 | 14b | 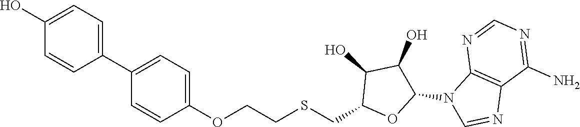 [SMILES: Nc1ncnc2c1ncn2[C@@H]1O[C@H](CSCCOc2ccc(-c3ccc(O)cc3)cc2)[C@@H](O)[C@H]1O] | 8 | 6 | 13 | 20 | 20 |
| | | NC1═NC═NC2═C1N═CN2[C@@H]3O[C@H](CSCCOC4═CC═ | | | | | |
| | | C(C5═C(O)C═C5)C═C4)[C@@H](O)[C@H]3O | | | | | |
| 1-160 | 14c | 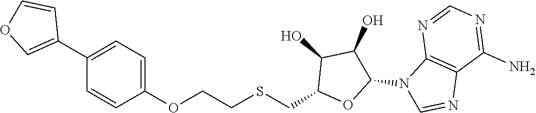 [SMILES: Nc1ncnc2c1ncn2[C@@H]1O[C@H](CSCCOc2ccc(-c3ccoc3)cc2)[C@@H](O)[C@H]1O] | 3 | 6 | 17 | 3 | 39 |
| | | NC1═NC═NC2═C1N═CN2[C@@H]3O[C@H](CSCCOC4═CC═ | | | | | |
| | | C(C5═COC═C5)C═C4)[C@@H](O)[C@H]3O | | | | | |
| 1-162 | 14d | 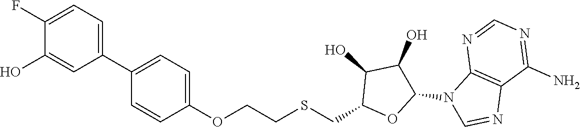 [SMILES: Nc1ncnc2c1ncn2[C@@H]1O[C@H](CSCCOc2ccc(-c3ccc(F)c(O)c3)cc2)[C@@H](O)[C@H]1O] | 16 | 3 | 51 | 18 | 42 |
| | | NC1═NC═NC2═C1N═CN2[C@@H]3O[C@H](CSCCOC4═ | | | | | |
| | | CC═C(C5═CC(O)═C(F)C═C5)C═C4)[C@@H](O)[C@H]3O | | | | | |
| 1-164 | 14e | 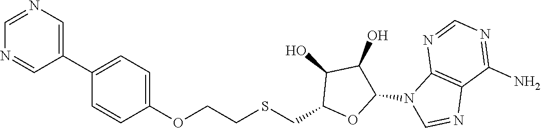 [SMILES: Nc1ncnc2c1ncn2[C@@H]1O[C@H](CSCCOc2ccc(-c3cncnc3)cc2)[C@@H](O)[C@H]1O] | 7 | 16 | 22 | 7 | 28 |
| | | NC1═NC═NC2═C1N═CN2[C@@H]3O[C@H](CSCCOC4═CC═ | | | | | |
| | | C(C5═CN═CN═C5)C═C4)[C@@H](O)[C@H]3O | | | | | |
| 1-166 | 14f | 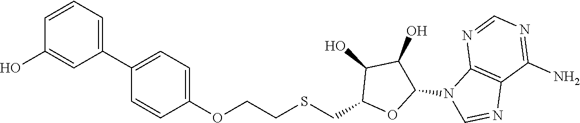 [SMILES: Nc1ncnc2c1ncn2[C@@H]1O[C@H](CSCCOc2ccc(-c3cccc(O)c3)cc2)[C@@H](O)[C@H]1O] | 5 | 5 | 62 | 13 | 40 |
| | | NC1═NC═NC2═C1N═CN2[C@@H]3O[C@H](CSCCOC4═CC═ | | | | | |
| | | C(C5═CC(O)═CC═C5)C═C4)[C@@H](O)[C@H]3O | | | | | |
| 1-167 | 14g | 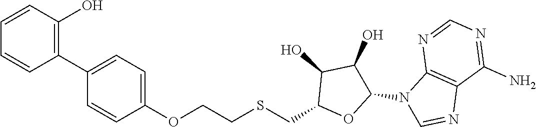 [SMILES: Nc1ncnc2c1ncn2[C@@H]1O[C@H](CSCCOc2ccc(-c3ccccc3O)cc2)[C@@H](O)[C@H]1O] | 5 | 5 | 35 | 10 | 39 |
| | | NC1═NC═NC2═C1N═CN2[C@@H]3O[C@H](CSCCOC4═CC═C(C5═ | | | | | |
| | | C(O)C═CC═C5)C═C4)[C@@H](O)[C@H]3O | | | | | |
| 1-168 | 14h | 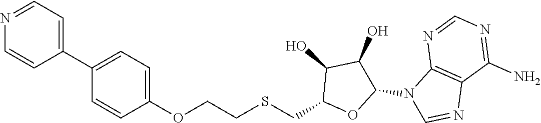 [SMILES: Nc1ncnc2c1ncn2[C@@H]1O[C@H](CSCCOc2ccc(-c3ccncc3)cc2)[C@@H](O)[C@H]1O] | 10 | 8 | 48 | 6 | 15 |
| | | NC1═NC═NC2═C1N═CN2[C@@H]3O[C@H](CSCCOC4═ | | | | | |
| | | CC═C(C5═CC═NC═C5)C═C4)[C@@H](O)[C@H]3O | | | | | |
| 1-83 | 19a | 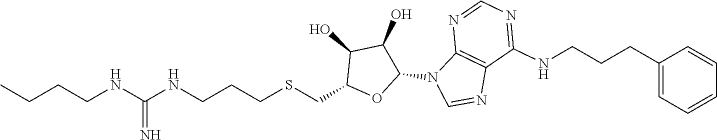 [SMILES: CCCCNC(=N)NCCCSC[C@H]1O[C@@H](n2cnc3c(NCCCc4ccccc4)ncnc32)[C@H](O)[C@@H]1O] | 94 | 14 | 47 | 84 | 85 |
| | | O[C@H]1[C@@H](O)[C@H](N2C═NC3═C2N═CN═ | | | | | |
| | | C3NCCCC4═CC═CC═C4)O[C@@H]1CSCCCNC(NCCCC)═N | | | | | |
| 1-84 | 19b | 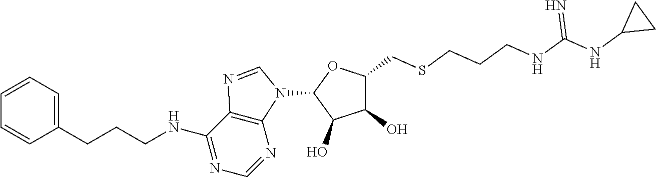 [SMILES: N=C(NCCCSC[C@H]1O[C@@H](n2cnc3c(NCCCc4ccccc4)ncnc32)[C@H](O)[C@@H]1O)NC1CC1] | 86 | 13 | 23 | 73 | 69 |
| | | O[C@H]1[C@@H](O)[C@H](N2C═NC3═C2N═CN═ | | | | | |
| | | C3NCCCC4═CC═CC═C4)O[C@@H]1CSCCCNC(NC5CC5)═N | | | | | |
| 1-85 | 19c | 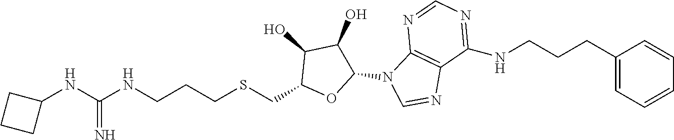 [SMILES: N=C(NCCCSC[C@H]1O[C@@H](n2cnc3c(NCCCc4ccccc4)ncnc32)[C@H](O)[C@@H]1O)NC1CCC1] | 80 | 8 | 50 | 93 | 91 |
| | | O[C@H]1[C@@H](O)[C@H](N2C═NC3═C2N═CN═C3NCCCC4═ | | | | | |
| | | CC═CC═C4)O[C@@H]1CSCCCNC(NC5CCC5)═N | | | | | |
| 1-86 | 19d | 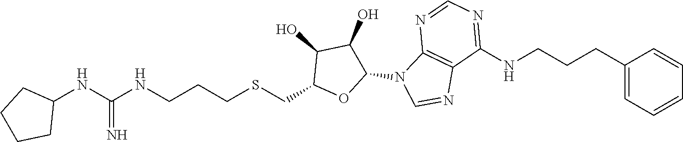 [SMILES: N=C(NCCCSC[C@H]1O[C@@H](n2cnc3c(NCCCc4ccccc4)ncnc32)[C@H](O)[C@@H]1O)NC1CCCC1] | 90 | 8 | 60 | 91 | 85 |
| | | O[C@H]1[C@@H](O)[C@H](N2C═NC3═C2N═CN═C3NCCCC4═ | | | | | |
| | | CC═CC═C4)O[C@@H]1CSCCCNC(NC5CCCC5)═N | | | | | |
| 1-87 | 19e | 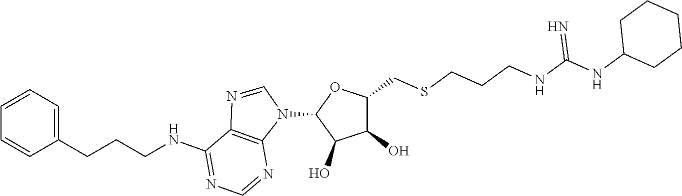 [SMILES: N=C(NCCCSC[C@H]1O[C@@H](n2cnc3c(NCCCc4ccccc4)ncnc32)[C@H](O)[C@@H]1O)NC1CCCCC1] | 89 | 10 | 44 | 88 | 79 |
| | | O[C@H]1[C@@H](O)[C@H](N2C═NC3═C2N═CN═C3NCCCC4═CC═ | | | | | |
| | | CC═C4)O[C@@H]1CSCCCNC(NC5CCCCC5)═N | | | | | |
| 1-88 | 19f | 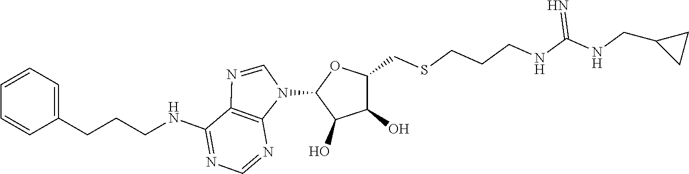 [SMILES: N=C(NCCCSC[C@H]1O[C@@H](n2cnc3c(NCCCc4ccccc4)ncnc32)[C@H](O)[C@@H]1O)NCC1CC1] | 76 | 3 | 36 | 88 | 74 |
| | | O[C@H]1[C@@H](O)[C@H](N2C═NC3═C2N═CN═C3NCCCC4═ | | | | | |
| | | CC═CC═C4)O[C@@H]1CSCCCNC(NCC5CC5)═N | | | | | |
| 1-89 | 19h | 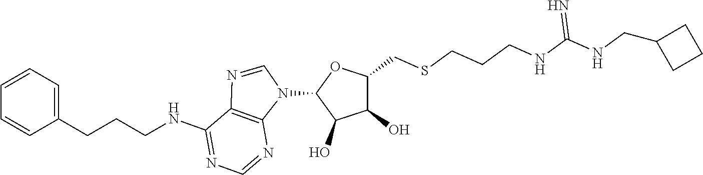 [SMILES: N=C(NCCCSC[C@H]1O[C@@H](n2cnc3c(NCCCc4ccccc4)ncnc32)[C@H](O)[C@@H]1O)NCC1CCC1] | 80 | 7 | 44 | 90 | 85 |
| | | O[C@H]1[C@@H](O)[C@H](N2C═NC3═C2N═CN═C3NCCCC4═ | | | | | |
| | | CC═CC═C4)O[C@@H]1CSCCCNC(NCC5CCC5)═N | | | | | |
| 1-92 | 19h | 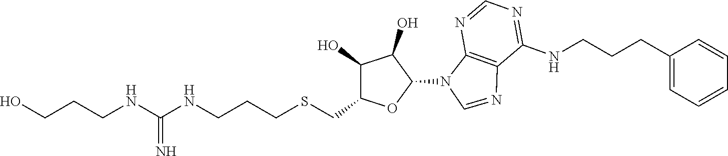 [SMILES: N=C(NCCCO)NCCCSC[C@H]1O[C@@H](n2cnc3c(NCCCc4ccccc4)ncnc32)[C@H](O)[C@@H]1O] | 64 | 10 | 54 | 84 | 55 |
| | | O[C@H]1[C@@H](O)[C@H](N2C═NC3═C2N═CN═C3NCCCC4═ | | | | | |
| | | CC═CC═C4)O[C@@H]1CSCCCNC(NCCCO)═N | | | | | |
| 1-93 | 19i | 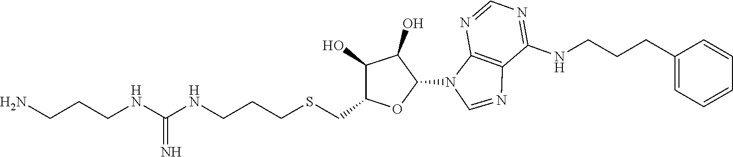 [SMILES: N=C(NCCCN)NCCCSC[C@H]1O[C@@H](n2cnc3c(NCCCc4ccccc4)ncnc32)[C@H](O)[C@@H]1O] | 89 | 13 | 37 | 79 | 74 |
| | | O[C@H]1[C@@H](O)[C@H](N2C═NC3═C2N═CN═C3NCCCC4═ | | | | | |
| | | CC═CC═C4)O[C@@H]1CSCCCNC(NCCCN)═N | | | | | |
| 1-94 | 19j | 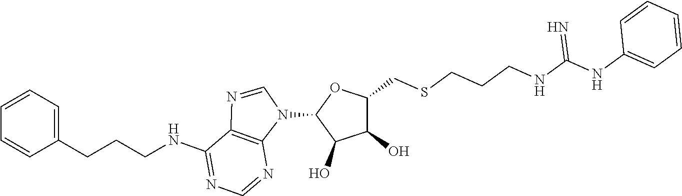 [SMILES: N=C(NCCCSC[C@H]1O[C@@H](n2cnc3c(NCCCc4ccccc4)ncnc32)[C@H](O)[C@@H]1O)Nc1ccccc1] | 83 | 2 | 54 | 92 | 86 |
| | | O[C@H]1[C@@H](O)[C@H](N2C═NC3═C2N═CN═C3NCCCC4═CC═CC═ | | | | | |
| | | C4)O[C@@H]1CSCCCNC(NC5═CC═CC═C5)═N | | | | | |
| 1-95 | 19k | 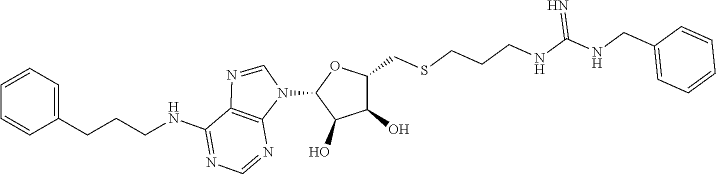 [SMILES: N=C(NCCCSC[C@H]1O[C@@H](n2cnc3c(NCCCc4ccccc4)ncnc32)[C@H](O)[C@@H]1O)NCc1ccccc1] | 89 | 10 | 44 | 89 | 83 |
| | | O[C@H]1[C@@H](O)[C@H](N2C═NC3═C2N═CN═C3NCCCC4═ | | | | | |
| | | CC═CC═C4)O[C@@H]1CSCCCNC(NCC5═CC═CC═C5)═N | | | | | |
| 1-96 | 19l | 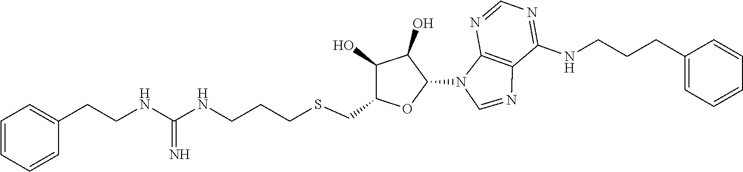 [SMILES: N=C(NCCCSC[C@H]1O[C@@H](n2cnc3c(NCCCc4ccccc4)ncnc32)[C@H](O)[C@@H]1O)NCCc1ccccc1] | 90 | 9 | 26 | 86 | 87 |
| | | O[C@H]1[C@@H](O)[C@H](N2C═NC3═C2N═CN═C3NCCCC4═CC═ | | | | | |
| | | CC═C4)O[C@@H]1CSCCCNC(NCCC5═CC═CC═C5)═N | | | | | |
| 1-97 | 19m | 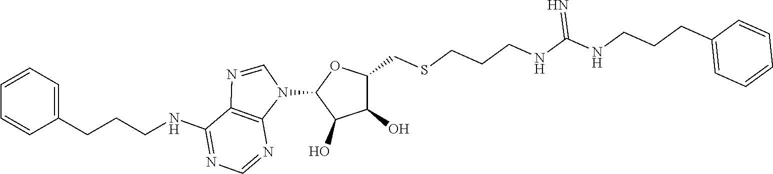 [SMILES: N=C(NCCCSC[C@H]1O[C@@H](n2cnc3c(NCCCc4ccccc4)ncnc32)[C@H](O)[C@@H]1O)NCCCc1ccccc1] | 87 | 5 | 58 | 83 | 85 |
| | | O[C@H]1[C@@H](O)[C@H](N2C═NC3═C2N═CN═C3NCCCC4═CC═ | | | | | |
| | | CC═C4)O[C@@H]1CSCCCNC(NCCCC5═CC═CC═C5)═N | | | | | |

Another aspect of the present disclosure are methods and strategies for creating synthetic PRMT inhibitors as described below. The selection of starting materials and focused changes as depicted in the figures and descriptions below broadly describe the methods and synthesis strategies of the current disclosure.

As used herein, the following terms and phrases shall have the meanings set forth below. Unless defined otherwise, all technical and scientific terms used herein have the same meaning as commonly understood to one of ordinary skill in the art.

The terms “substituted,” “substituent,” and “functional group” refer to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups.

Examples of branched alkyl groups include, but are not limited to, isopropyl, isobutyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carboncarbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein. “Aryl” and the phrase “aryl group” include groups of the formula:

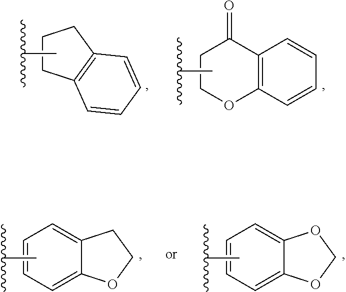

each of which can be substituted or unsubstituted, such as hydroxy substituted.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl.

Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). As used herein, “benzyl” is used to specifically identify a 6 membered carbon ring (as in benzene) which may be additionally optionally substituted.

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃*, wherein each R is independently selected, and protonated forms of each, except for —NR₃*, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the present disclosure described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the present disclosure described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing

the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles. Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol. In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

The following enumerated Embodiments are presented herein for the sake of completeness:

A compound having a formula (I):

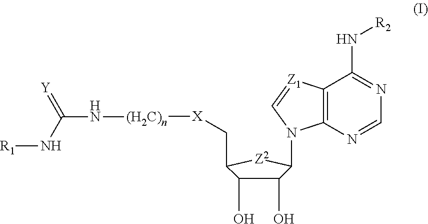

or a pharmaceutically acceptable salt thereof, wherein:
X is a N or S,
n is an integer from 1-12; and
Y is NR^{p} or O, wherein R^{p} is H or a protecting group, and
Z₁ is N or CH, and
Z₂ is O or CH₂, and
R₁ is an acyl, alkyl, alkenyl, alkynyl, heteroalkyl, heteroalkenyl, heteroalkynyl,
   cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl.

The compound of Embodiment 1, or a pharmaceutically acceptable salt thereof, wherein R₁ is CH₃.

The compound of Embodiment 1, or a pharmaceutically acceptable salt thereof, wherein R₂ is H.

The compound of Embodiment 1, or a pharmaceutically acceptable salt thereof, wherein Z₂ is O.

The compound of Embodiment 1, or a pharmaceutically acceptable salt thereof, wherein Z₂ is CH₂.

The compound of Embodiment 1, or a pharmaceutically acceptable salt thereof, wherein X is S.

The compound of Embodiment 1, or a pharmaceutically acceptable salt thereof, wherein Y is NH.

The compound of Embodiment 1, or a pharmaceutically acceptable salt thereof, wherein Y is O.

The compound of Embodiment 1, or a pharmaceutically acceptable salt thereof, wherein Z₁ is N.

The compound of Embodiment 1, or a pharmaceutically acceptable salt thereof, wherein R₁ and/or R₂ is selected from the group consisting of:

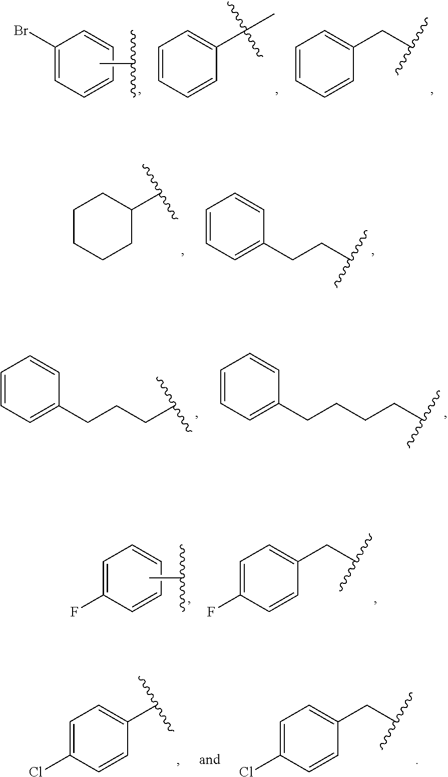

The compound of Embodiment 1, wherein the compound has the formula:

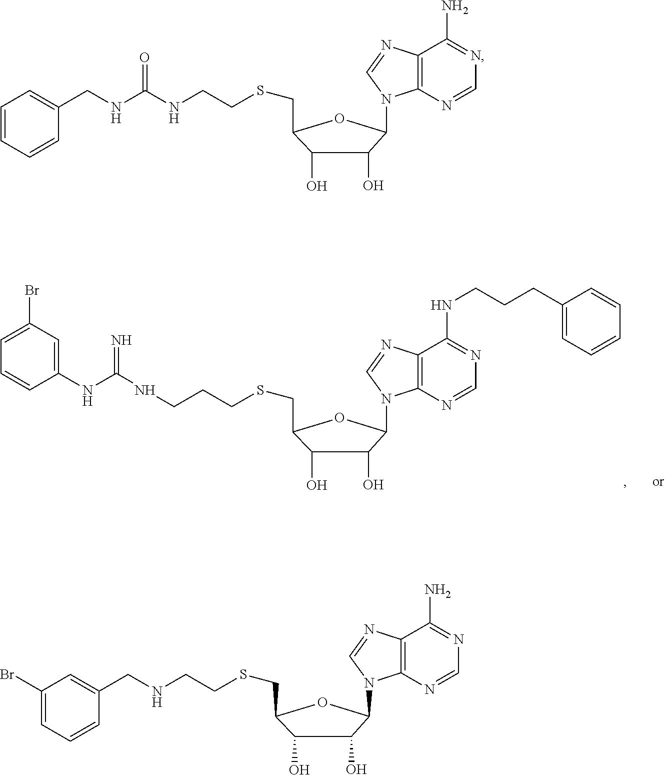 [SMILES: Nc1ncnc2c1ncn2C1OC(CSCCNC(=O)NCc2ccccc2)C(O)C1O ; N=C(NCCCSCC1OC(n2cnc3c(NCCCc4ccccc4)ncnc32)C(O)C1O)Nc1cccc(Br)c1 ; Nc1ncnc2c1ncn2[C@@H]1O[C@H](CSCCNCc2cccc(Br)c2)[C@@H](O)[C@H]1O]

or a pharmaceutically acceptable salt thereof.

The compound of Embodiment 1, wherein the compound has a formula (II):

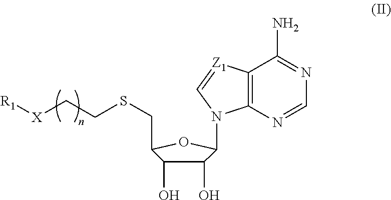

or a pharmaceutically acceptable salt thereof,
wherein:
n is an integer from 0-12,
Z₁ is N or CH, and
X is O or NH and
R₁ is an aryl or heterocyclic. R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The compound of Embodiment 12, wherein the compound has the formula (IIa):

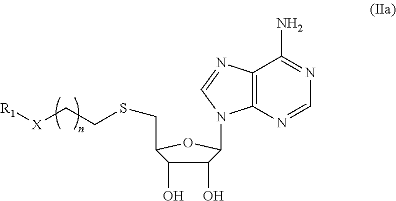

or a pharmaceutically acceptable salt thereof,
wherein:
n is an integer from 0-12,
X is O or NH and
R₁ is an aryl or heterocyclic.

The compound of Embodiment 12, wherein the compound has the formula:

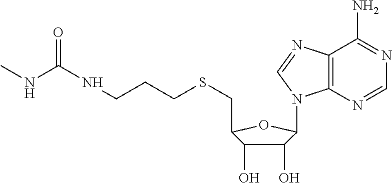 [SMILES: CNC(=O)NCCCSCC1OC(n2cnc3c(N)ncnc32)C(O)C1O]

or a pharmaceutically acceptable salt thereof.

A compound having the formula (III), wherein the compound has the formula (III):

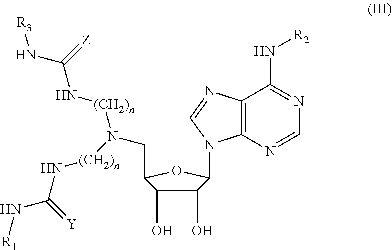

or a pharmaceutically acceptable salt thereof,
wherein:
each n is independently an integer from 1-12; and
Y and Z are either NH or O, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The compound of Embodiment 15, wherein the compound has the formula (IIIa):

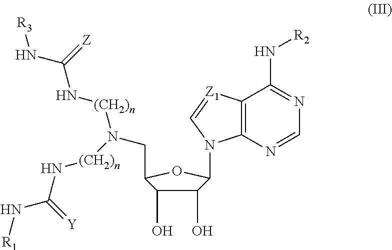

or a pharmaceutically acceptable salt thereof,
wherein:
each n is independently an integer from 1-12; and
Y and Z are either NH or O, and
Z₁ is N or CH, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

The compound of Embodiment 15, wherein the compound has the formula:

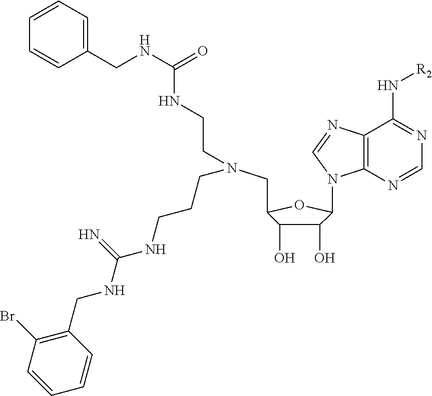

or a pharmaceutically acceptable salt thereof.

The compound of Embodiment 1, 12, 13, 15, or 16, wherein each n is, independently, an integer from 1 to 5, 1 to 3, 2 to 3, 5 to 10, 4 to 8, or 3 to 10.

The compound of Embodiment 15 or 16, wherein Y is NH.

The compound of Embodiment 15 or 16, wherein Z is O.

The compound of Embodiment 15 or 16, wherein Y is O.

The compound of Embodiment 15 or 16, wherein Z is NH.

The compound of Embodiment 15 or 16, wherein R₁ and R₃ are each, independently:

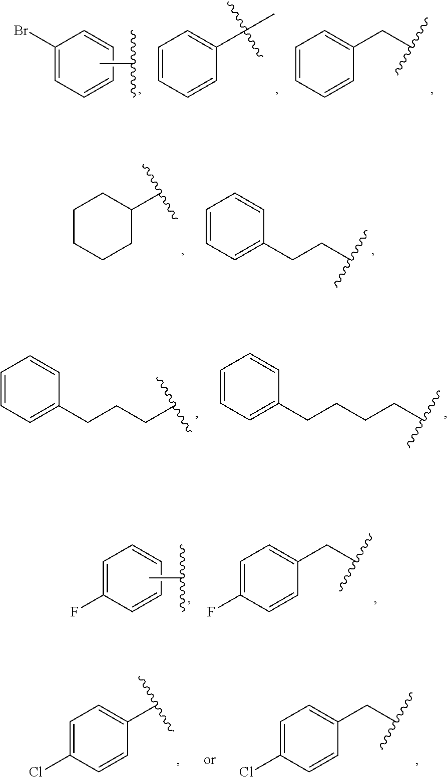

each of which can be further substituted.

The compound of Embodiment 1, 12, 13, 15, or 16, wherein R₁ is:

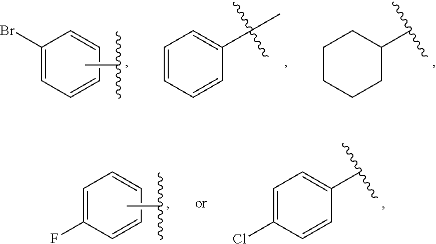

each of which can be further substituted.

The compound of Embodiment 15 or 16, wherein R₃ is:

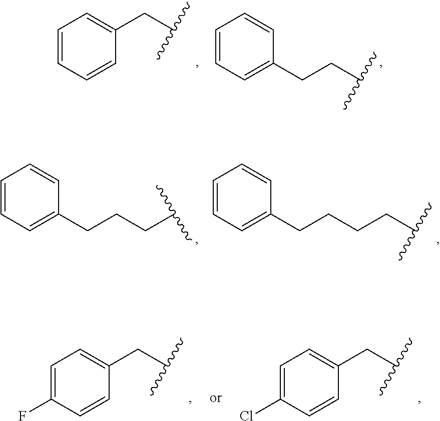

each of which can be further substituted.

The compound of Embodiment 15 or 16, wherein R₁ and R₃ are each, independently,

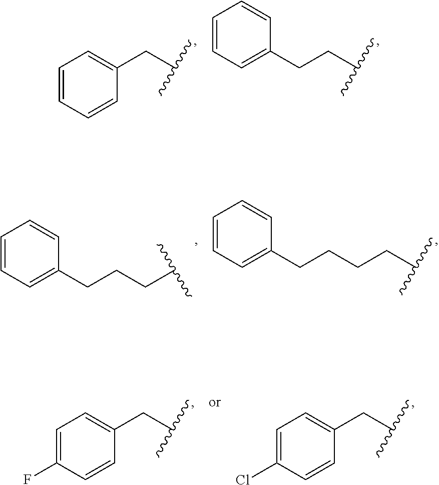

each of which can be further substituted.

The compound of Embodiment 15 or 16, wherein R₃ is:

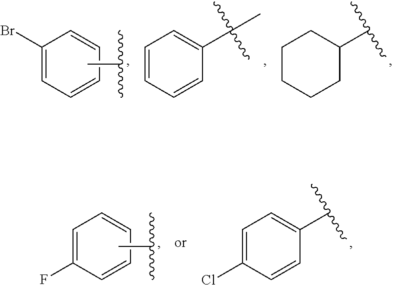

each of which can be further substituted.

The compound of Embodiment 1, 12, 13, 15, or 16, wherein R₁ is:

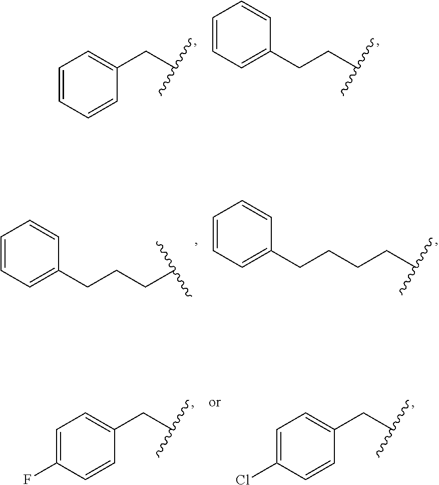

each of which can be further substituted.

A compound of the formula:

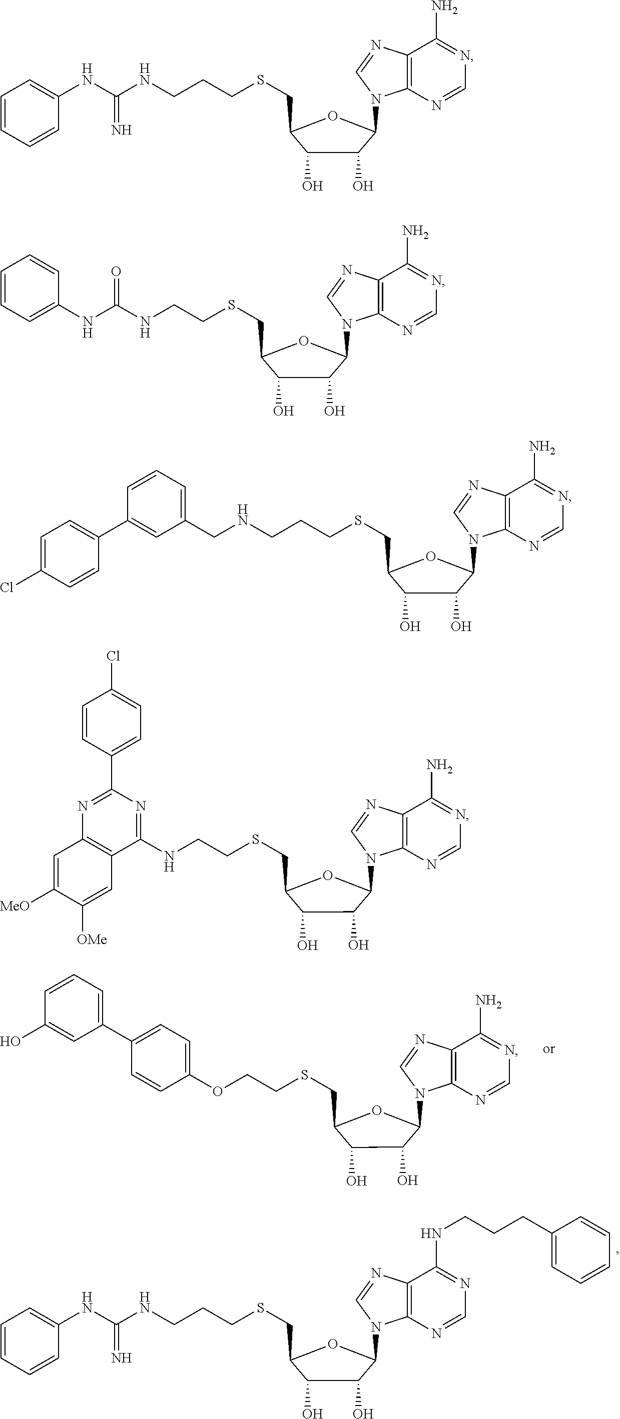

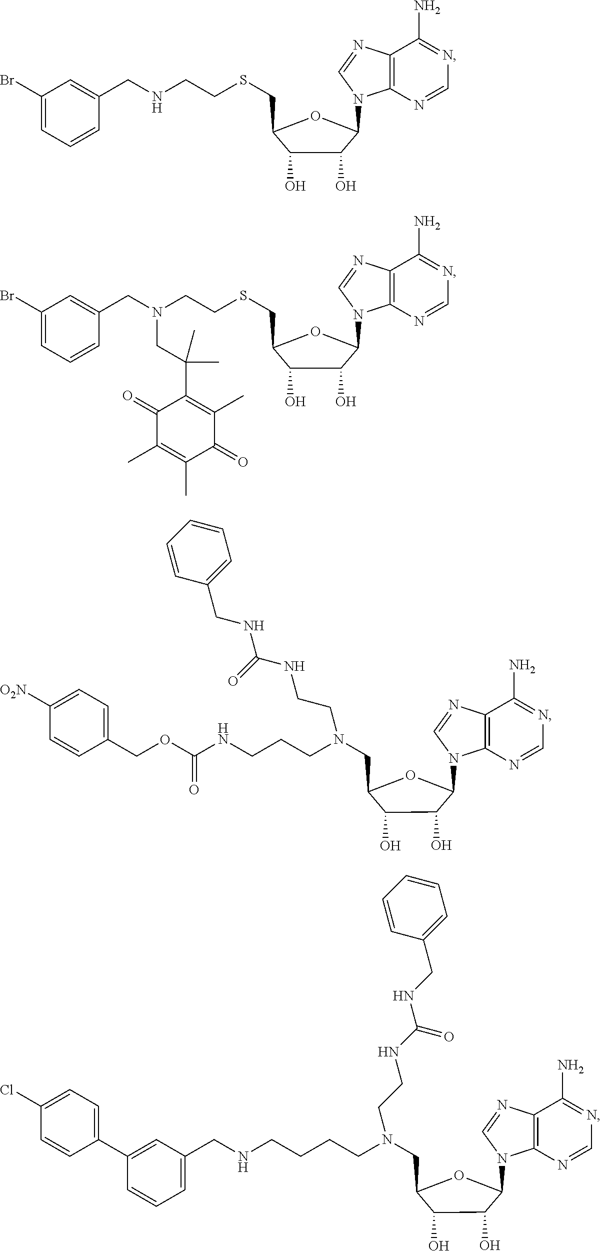

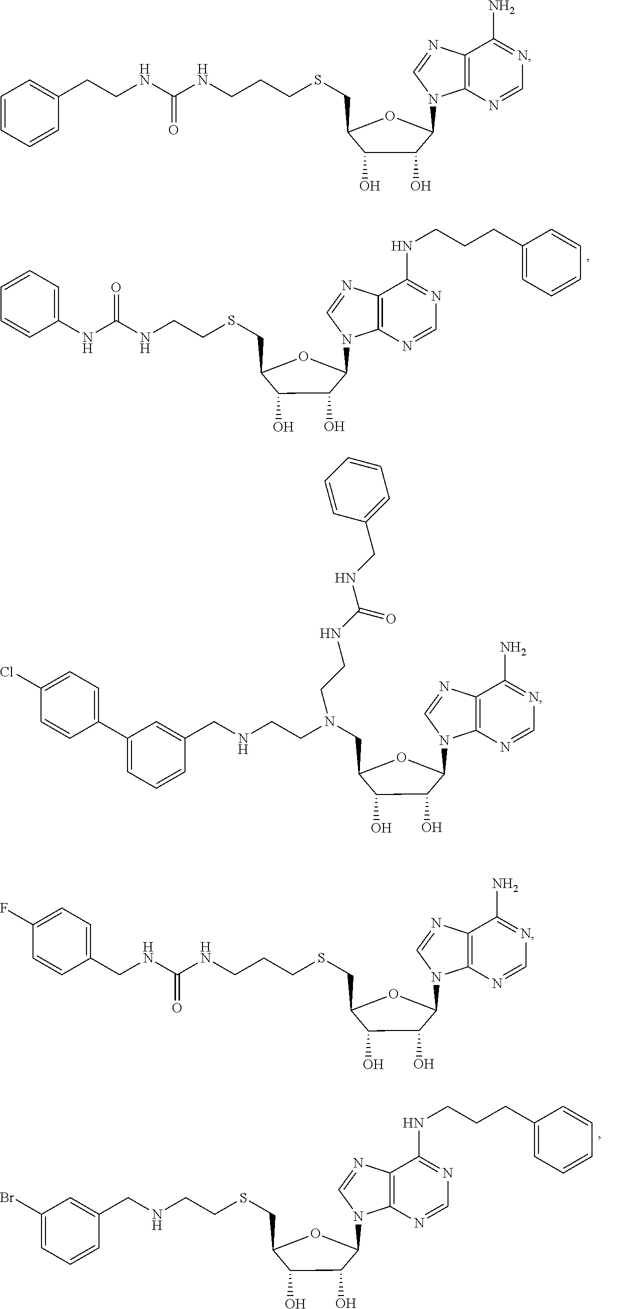

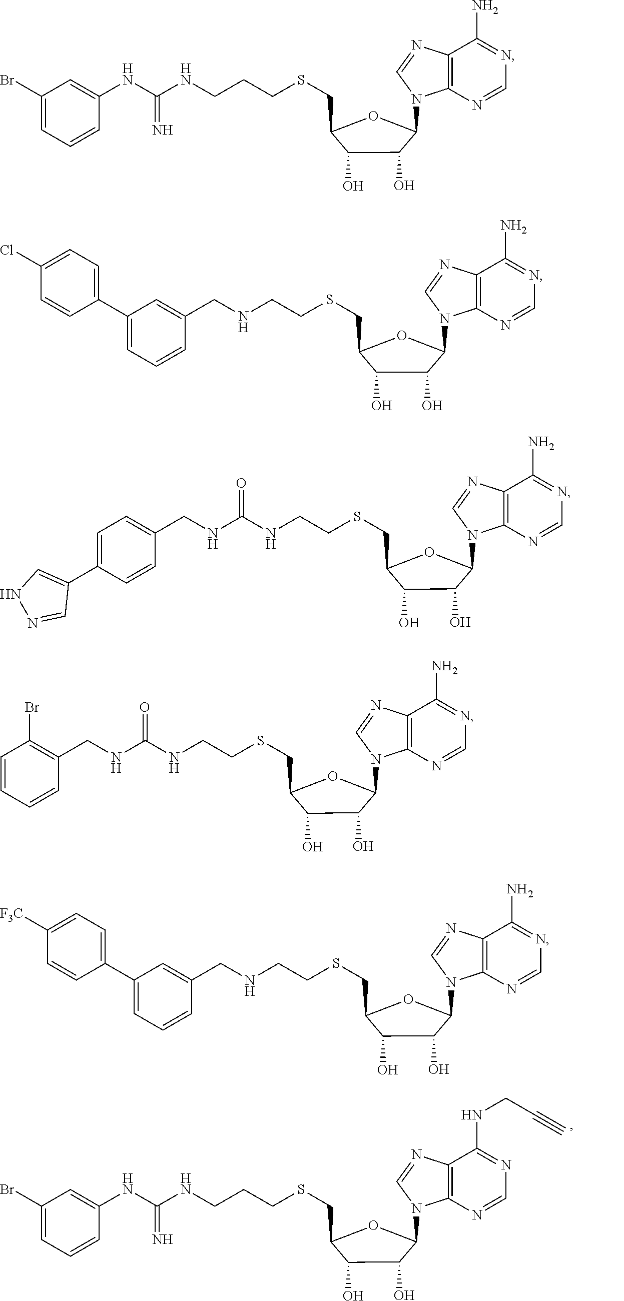 [SMILES: N=C(NCCCSC[C@H]1O[C@@H](n2cnc3c(N)ncnc32)[C@H](O)[C@@H]1O)Nc1cccc(Br)c1 ; Nc1ncnc2c1ncn2[C@@H]1O[C@H](CSCCNCc2cccc(-c3ccc(Cl)cc3)c2)[C@@H](O)[C@H]1O ; Nc1ncnc2c1ncn2[C@@H]1O[C@H](CSCCNC(=O)NCc2ccc(-c3cn[nH]c3)cc2)[C@@H](O)[C@H]1O ; Nc1ncnc2c1ncn2[C@@H]1O[C@H](CSCCNC(=O)NCc2ccccc2Br)[C@@H](O)[C@H]1O ; Nc1ncnc2c1ncn2[C@@H]1O[C@H](CSCCNCc2cccc(-c3ccc(C(F)(F)F)cc3)c2)[C@@H](O)[C@H]1O ; C#CCNc1ncnc2c1ncn2[C@@H]1O[C@H](CSCCCNC(=N)Nc2cccc(Br)c2)[C@@H](O)[C@H]1O]

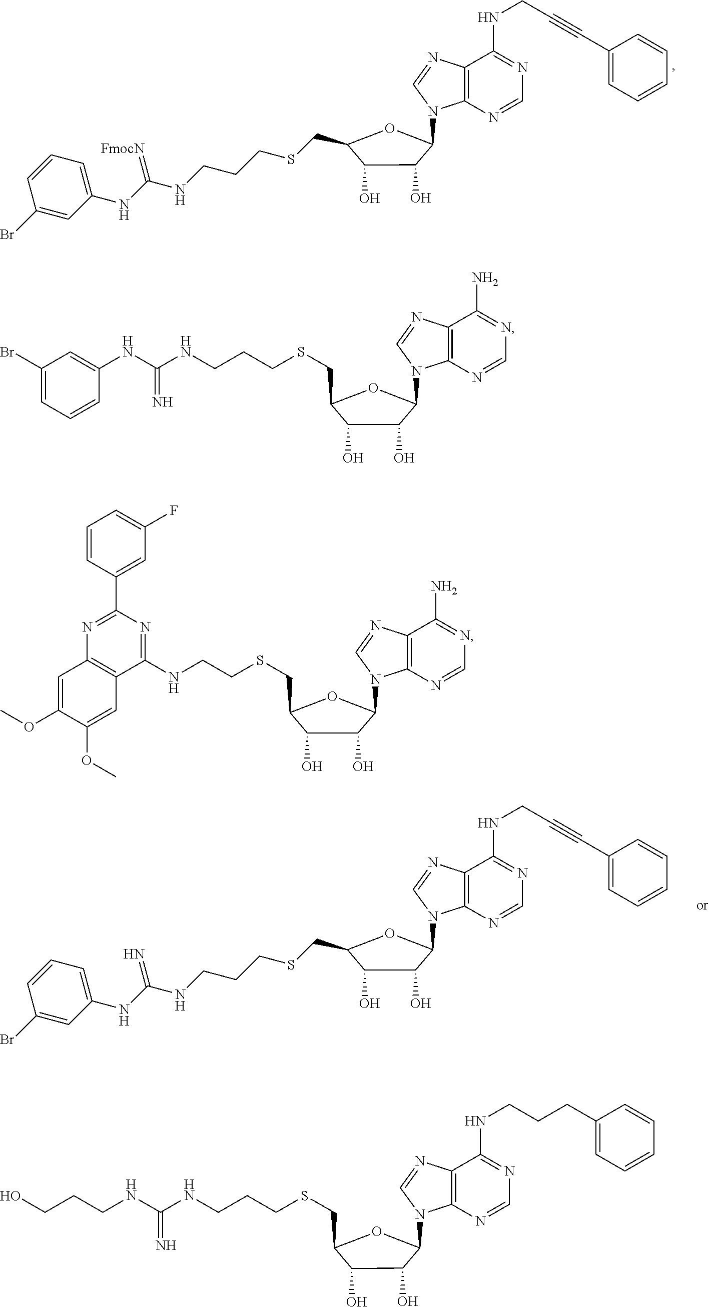

or a pharmaceutically acceptable salt thereof.

A compound of the formula:

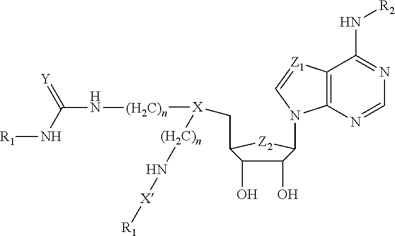

or a pharmaceutically acceptable salt thereof, wherein:
X is a N or S,
X′ is a NH, S or O,
n is an integer from 1-12; and
Y is NR^{p} or O, wherein R^{p} is H or a protecting group, and
Z₁ is N or CH, and
Z₂ is O or CH₂, and
each R₁ is, independently, an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl.

The compound of Embodiment 30, or a pharmaceutically acceptable salt thereof, wherein R₁ is CH₃.

The compound of Embodiment 30, or a pharmaceutically acceptable salt thereof, wherein R₂ is H.

The compound of Embodiment 30, or a pharmaceutically acceptable salt thereof, wherein Z₂ is O.

The compound of Embodiment 30, or a pharmaceutically acceptable salt thereof, wherein Z₂ is CH₂.

The compound of Embodiment 30, or a pharmaceutically acceptable salt thereof, wherein X is S.

The compound of Embodiment 30, or a pharmaceutically acceptable salt thereof, wherein Y is NH.

The compound of Embodiment 30, or a pharmaceutically acceptable salt thereof, wherein Y is O.

The compound of Embodiment 30, or a pharmaceutically acceptable salt thereof, wherein Z₁ is N.

The compound of Embodiment 30, or a pharmaceutically acceptable salt thereof, wherein each R₁ and/or R₂ is independently selected from the group consisting of:

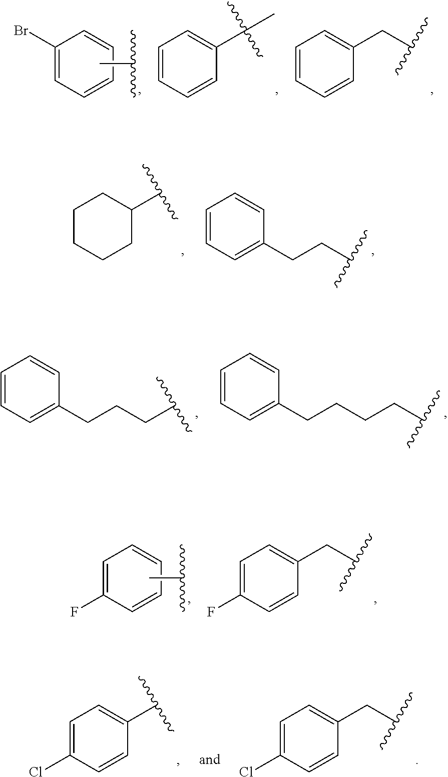

The compound of Embodiment 30, or a pharmaceutically acceptable salt thereof, wherein the compound is:

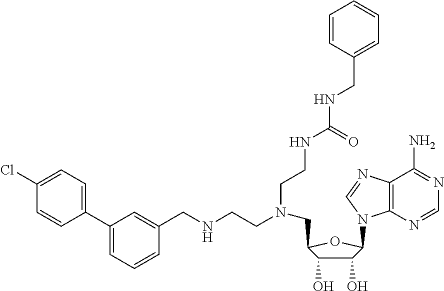 [SMILES: Nc1ncnc2c1ncn2[C@@H]1O[C@H](CN(CCNCc2cccc(-c3ccc(Cl)cc3)c2)CCNC(=O)NCc2ccccc2)[C@@H](O)[C@H]1O]

or a pharmaceutically acceptable salt thereof.

A pharmaceutical composition comprising one or more compounds of Embodiment 1, 12, 13, 15, 16, 18, or 30, or a pharmaceutically acceptable salt thereof, together with one or more diluents, excipients or carriers.

A pharmaceutical composition comprising one or more compounds of Embodiment 1, 12, 13, 15, 16, 18, or 30, or a pharmaceutically acceptable salt thereof, in combination with one or more other compounds by the same or different mode of action, together with one or more diluents, excipients or carriers.

A method for inhibiting PRMT activity comprising the step of administering to a mammal in need of relief from the effects of PRMT activity a therapeutically effective amount of one or more compounds of Embodiment 1, 12, 13, 15, 16, 18, or 30 or a pharmaceutically acceptable salt thereof.

The method of Embodiment 43, wherein the PRMT activity that is inhibited is the PRMT activity of PRMT1, PRMT3, PRMT4, PRMT5, PRMT6 or PRMT8.

The method of Embodiment 43, wherein the one or more compounds is selective for PRMT1.

The method of Embodiment 43, wherein the PRMT activity that is inhibited is the PRMT activity of PRMT3.

The method of Embodiment 43, wherein the one or more compounds is selective for PRMT4.

The method of Embodiment 43, wherein the one or more compounds is selective for PRMT5.

The method of Embodiment 43, wherein the PRMT activity that is inhibited is the PRMT activity of PRMT6.

The method of Embodiment 43, wherein the PRMT activity that is inhibited is the PRMT activity of PRMT8.

The method of Embodiment 43, wherein the inhibition of PRMT4 is at least 1000-fold (e.g., 1000-fold to 2000-fold, 1500-fold to 3000-fold, or 1000-fold to 3000-fold) over other MTases.

The method of Embodiment 43, wherein the inhibition of PRMT3/4/5 is at least 30-fold (e.g. from 30-fold to 100 fold, 50-fold to 75-fold, or 30-fold to 90-fold) to over PRMT1 and 8.

The method of Embodiment 43, wherein the inhibition of PRMT1/3/4/6/8 is at least 100-fold (e.g., 100-fold to 200-fold, 150-fold to 300-fold, or 100-fold to 300-fold) over other MTases.

The method of Embodiment 43, wherein the inhibition of PRMT4 is at least 50-fold (e.g., 50-fold to 500 fold, 50-fold- to 100-fold, 75-fold to 150-fold or 100-fold to 300-fold) over other MTases.

The method of Embodiment 43, wherein the inhibition of PRMT5 is a least 1,000-fold (e.g., 1000-fold to 2000-fold, 1500-fold to 3000-fold, or 1000-fold to 3000-fold) for PRMT5 over other MTases.

The method of Embodiment 43, wherein the inhibition of PRMT4 is at least 100-fold (e.g., 100-fold to 200-fold, 150-fold to 300-fold, or 100-fold to 300-fold) over other MTAses.

The method of Embodiment 43, wherein the inhibition of PRMT4/7 is at least 100-fold (e.g., 100-fold to 200-fold, 150-fold to 300-fold, or 100-fold to 300-fold) for PRMT4/7 over other MTases.

The method of Embodiment 51, wherein the compound that is administered is YD1130.

The method of Embodiment 52, wherein the compound that is administered is YD1113.

The method of Embodiment 53, wherein the compound that is administered is YD1290.

The method of Embodiment 54, wherein the compound that is administered is YD1349.

The method of Embodiment 55, wherein the compound that is administered is YD1195.

The method of Embodiment 56, wherein the compound that is administered is AK442.

The method of Embodiment 57, wherein the compound that is administered is AK447 or YD1133.

### Examples

The disclosure can be better understood by reference to the following examples which are offered by way of illustration. The disclosure is not limited to the examples given herein.

### 1.0 Synthesis and Characterization of the Target Compounds

Starting materials, reagents, and solvents were obtained from commercial sources. Analytical and preparative high-pressure liquid chromatography (RP-HPLC) was performed on Agilent 1260 Series system. Systems were run with a 5-95% acetonitrile/water gradient with a 0.1% TFA. The peptides were synthesized using a Liberty Automated Microwave Peptide Synthesizer (CEM) with the manufacturer's standard coupling cycles at 0.1 mmol scales and cleaved from the resin in a cocktail of TFA/2,2′-(ethylenedioxy)diethanethiol/H₂O/triisopropylsilane (94:2.5:2.5:1) and confirmed by MS. All 1H and 13C NMR spectra were recorded on a Brucker 500 MHz spectrometer. LRMS spectra were recorded on an Agilent 6470 quadrupole LCMS instrument. HRMS spectra were recorded on an Agilent high-resolution 6550quadrupole time-of-flight (Q-TOF) LC-MS instrument. All compounds used for their IC₅₀ determination possessed a purity of over 95%, based on HPLC analysis.

### Synthesis of 2′,3′-O-isopropylideneadenosine

To a stirring solution of adenosine (13.36 g, 50 mmol) in 1.5 L of dry acetone was added p-toluenesulfonic acid monohydrate (10.46 g, 55 mmol) and triethyl orthoformate (42 mL, 250 mmol). The mixture was stirred at room temperature overnight and then neutralized with a saturated aqueous solution of ammonium-hydroxide. The volatiles were removed under reduced pressure and the residues were cooled to crystallize. After filtration, 2′,3′-O-isopropylideneadenosine was obtained as a white solid (13.66 g, 89% yield). LRMS (ESI) m/z calcd for C₁₃H₁₇N₅O₄ [M+H]+: 308.13; Found: 308.12.

### Synthesis of 2

To an ice-cold solution of triphenylphosphine (3.67 g, 14.0 mmol) in dry THE (100 mL) was added diethyl azodicarboxylate (2.2 mL, 14 mmol) dropwise over 5 min. After stirring for 30 min, 2′,3′-O-Isopropylideneadenosine (2.15 g, 7.0 mmol) was added and the reaction continued for 2 h. To the resulting yellow suspension was added a solution of thioacetic acid (1.0 mL, 14.0 mmol) in dry THE (5 mL) dropwise and stirred overnight with the temperature slowly increased to room temperature.

After the reaction was completed, the volatiles were removed under reduced pressure to afford crude oil, which was subsequently purified by flash chromatography (DCM/MeOH, 97/3) to afford 2 (2.32 g, 91% yield). LRMS (ESI) m/z calcd for C15H19N5O4S [M+H]+: 366.12; Found: 366.10.

### Synthesis of 3a and 3b

To a stirring solution of compound 2 (1095 mg, 3.0 mmol) and tert-butyl (2-bromoethyl)carbamate or tert-butyl (3-bromopropyl)carbamate (4.5 mmol) in dry methanol (50 mL) was added sodium methoxide (30% in methanol, 1.62 mL, 9.0 mmol) under an argon atmosphere. After overnight, the volatiles were removed under reduced pressure and the residue was purified by flash chromatography (DCM/MeOH, 97/3) to afford 3a (1160 mg, 83% yield) and 3b (1267 mg, 88% yield) as a colorless oil. 3a, LRMS (ESI) m/z calcd for C20H30N6O5S [M+H]+: 467.21; Found: 467.18. 3b, LRMS (ESI) m/z calcd for C21H32N6O5S [M+H]+: 481.22; Found: 481.19.

### Synthesis of 4a and 4b

To a cooled suspension of 3a or 3b (0.1 mmol) in water (1 mL), TFA (4 mL) was added dropwise at 0° C. The mixture was stirred for 24 h at room temperature and then under lyophilization to offer a pale-yellow solid in quantitative yield. 4a, LRMS (ESI) m/z calcd for C15H22N5O3S [M+H]+: 367.15; Found 367.12. 4b, LRMS (ESI) m/z calcd for C16H24N6O3S [M+H]+: 381.17; Found 381.17.

### Synthesis of Compound 1a (II710)

Compound 1a (II710) was synthesized with reported methods. (lyamu et al. 2021)

### Synthesis of compound 1d

To a stirring solution of 4a (163 mg, 0.5 mmol) in 5 mL anhydrous DMF was added DIPEA (194 mg, 1.5 mmol) and 1-Fmoc-3-benzylthiourea (388 mg, 1.0 mmol) at room temperature. The reaction mixture was stirred at room temperature overnight. Then the mixture was diluted with 100 mL H2O and extracted with EtOAc (3×100 mL). The combined organic layers were washed with brine and dried over anhydrous sodium sulfate. The volatiles were removed under reduced pressure to afford an oil, which was dissolved in DCM/piperidine (10/3 mL) and stirred at room temperature for 1 h. Next, the volatiles were removed under reduced pressure to generate oil. The crude oil was dissolved in 5 mL methanol and separated with prep-HPLC (MeCN/H2O) to afford 1d (116 mg, 51% yield). MS (ESI) m/z calcd for C20H26N8O3S [M+H]+: 459.1922; Found: 459.1934. 1H NMR (500 MHz, CD3OD) δ 8.45 (s, 1H), 8.36 (s, 1H), 7.35 (t, J=1.1 Hz, 1H), 7.31-7.27 (m, 4H), 6.05 (d, J=4.7 Hz, 1H), 4.72 (d, J=5.0 Hz, 1H), 4.40 (s, 2H), 4.32 (t, J=5.1 Hz, 1H), 4.21 (dt, J=6.7, 5.0 Hz, 1H), 3.42 (t, J=6.6 Hz, 2H), 3.05-2.92 (m, 2H), 2.78 (t, J=6.7 Hz, 2H). 13C NMR (126 MHz, CD3OD) δ 156.20, 151.84, 148.72, 145.99, 142.12, 136.22, 128.53, 128.51, 128.11, 127.62, 126.87, 119.24, 89.14, 84.42, 73.76, 72.59, 44.60, 40.85, 33.85, 31.40.

### General Procedure for the Synthesis of 1b-1c and 1e-1m

To a stirring solution of 4a (163 mg, 0.5 mmol) or 4b (170 mg, 0.5 mmol) in 5 mL anhydrous DMF was added TEA (152 mg, 1.5 mmol) and isocyanate (0.6 mmol) at room temperature. The reaction mixture was stirred at room temperature for 1-4 h. At the end of the reaction, the mixture was diluted with 100 mL Et2O and centrifuged. Next, the residue was dissolved in 5 mL methanol and then subjected to prep-HPLC (MeCN/H2O) to afford 1b-1c and 1e-1m (53-88% yield).

### 1-(3-((((2S,3S,4R,5R)-5-(6-amino-9H-purin-9-yl)-3,4-dihydroxytetrahydrofuran-2-yl)methyl)thio)propyl)-3-benzylurea (1b)

White solid 1b (189 mg, 80% yield). HRMS (ESI) m/z calcd for C21H27N7O4S [M+H]+: 474.1918; Found: 474.1922. 1H NMR (500 MHz, CD3OD) δ 8.51 (s, 1H), 8.37 (s, 1H), 7.30-7.21 (m, 4H), 7.20-7.13 (m, 1H), 6.06 (d, J=5.0 Hz, 1H), 4.73 (t, J=5.2 Hz, 1H), 4.32 (t, J=4.9 Hz, 1H), 4.28 (s, 2H), 4.23 (td, J=5.8, 4.4 Hz, 1H), 3.19 (td, J=6.8, 1.3 Hz, 2H), 3.01-2.84 (m, 2H), 2.59 (t, J=7.3 Hz, 2H), 1.74 (p, J=6.8 Hz, 2H). 13C NMR (126 MHz, CD3OD) δ 159.77, 150.77, 148.62, 144.37, 142.61, 139.87, 128.07, 126.75, 126.70, 126.57, 119.16, 89.03, 84.54, 73.96, 72.52, 43.33, 38.59, 33.93, 29.89, 29.71.

### 1-(2-((((2S,3S,4R,5R)-5-(6-amino-9H-purin-9-yl)-3,4-dihydroxytetrahydrofuran-2-yl)methyl)thio)ethyl)-3-benzylurea (1c)

White solid 1c (145 mg, 63% yield). HRMS (ESI) m/z calcd for C20H25N7O4S [M+H]+: 460.1762; Found: 460.1776. 1H NMR (500 MHz, CD3OD) δ 8.50 (s, 1H), 8.36 (s, 1H), 7.33-7.23 (m, 4H), 7.22-7.14 (m, 1H), 6.05 (d, J=5.0 Hz, 1H), 4.72 (t, J=5.2 Hz, 1H), 4.31 (t, J=4.9 Hz, 1H), 4.29 (s, 2H), 4.22 (q, J=5.8 Hz, 1H), 3.32 (d, J=7.2 Hz, 2H), 3.05-2.91 (m, 2H), 2.68 (td, J=6.8, 4.0 Hz, 2H). 13C NMR (126 MHz, CD3OD) δ 159.54, 151.31, 148.71, 145.19, 142.37, 139.86, 128.03, 126.72, 126.54, 119.22, 89.04, 84.68, 73.91, 72.52, 43.27, 39.40, 33.76, 32.80.

### 1-(2-((((2S,3S,4R,5R)-5-(6-amino-9H-purin-9-yl)-3,4-dihydroxytetrahydrofuran-2-yl)methyl)thio)ethyl)-3-(4-fluorobenzyl)urea (1e)

White solid 1e (143 mg, 60% yield). HRMS (ESI) m/z calcd for C20H24FN7O4S [M+H]+: 478.1667; Found: 478.1672. 1H NMR (500 MHz, CD3OD) δ 8.50 (s, 1H), 8.38 (s, 1H), 7.25 (dd, J=8.8, 5.3 Hz, 2H), 6.97 (t, J=8.8 Hz, 2H), 6.06 (d, J=4.9 Hz, 1H), 4.72 (t, J=5.1 Hz, 1H), 4.32 (t, J=4.9 Hz, 1H), 4.26 (s, 2H), 4.22 (d, J=5.3 Hz, 1H), 3.32-3.28 (m, 2H), 3.04-2.91 (m, 2H), 2.67 (td, J=6.7, 2.7 Hz, 2H). 13C NMR (126 MHz, CD3OD) δ 162.82, 159.51, 150.94, 148.62, 144.63, 142.54, 135.95, 128.61, 128.55, 119.16, 114.69, 114.52, 89.05, 84.61, 73.97, 72.52, 42.57, 39.40, 33.79, 32.80.

### 1-(2-((((2S,3S,4R,5R)-5-(6-amino-9H-purin-9-yl)-3,4-dihydroxytetrahydrofuran-2-yl)methyl)thio)ethyl)-3-phenethylurea (1f)

White solid 1f (154 mg, 65% yield). HRMS (ESI) m/z calcd for C21H27N7O4S [M+H]+: 474.1918; Found 474.1920. 1H NMR (500 MHz, CD3OD) δ 8.49 (s, 1H), 8.36 (s, 1H), 7.22 (t, J=7.4 Hz, 2H), 7.19-7.07 (m, 3H), 6.05 (d, J=5.0 Hz, 1H), 4.73 (t, J=5.2 Hz, 1H), 4.32 (t, J=4.9 Hz, 1H), 4.23 (q, J=5.9 Hz, 1H), 3.31 (t, J=7.3 Hz, 2H), 3.27 (t, J=6.9 Hz, 2H), 3.05-2.90 (m, 2H), 2.72 (t, J=7.2 Hz, 2H), 2.64 (td, J=6.8, 2.8 Hz, 2H). 13C NMR (126 MHz, CD3OD) δ 159.56, 151.01, 148.63, 144.76, 142.50, 139.33, 128.45, 128.06, 125.86, 119.15, 89.02, 84.60, 73.95, 72.53, 41.27, 39.39, 36.10, 33.79, 32.75.

### 1-(2-((((2S,3S,4R,5R)-5-(6-amino-9H-purin-9-yl)-3,4-dihydroxytetrahydrofuran-2-yl)methyl)thio)ethyl)-3-(3-phenylpropyl)urea (1g)

White solid 1g (190 mg, 78% yield). HRMS (ESI) m/z calcd for C22H29N7O4S [M+H]+: 488.2075; Found: 488.2077. 1H NMR (500 MHz, CD3OD) δ 8.50 (s, 1H), 8.36 (s, 1H), 7.25-7.17 (m, 2H), 7.16-7.07 (m, 3H), 6.05 (d, J=5.0 Hz, 1H), 4.71 (t, J=5.1 Hz, 1H), 4.32 (t, J=4.9 Hz, 1H), 4.23 (td, J=5.9, 4.5 Hz, 1H), 3.28 (d, J=6.8 Hz, 2H), 3.10 (t, J=6.9 Hz, 2H), 3.05-2.89 (m, 2H), 2.66 (td, J=6.8, 3.1 Hz, 2H), 2.58 (dd, J=8.7, 6.8 Hz, 2H), 1.74 (dtd, J=9.4, 7.5, 6.5 Hz, 2H). 13C NMR (126 MHz, CD3OD) δ 160.93, 150.77, 148.59, 144.30, 142.59, 141.72, 127.97, 127.96, 125.41, 119.17, 117.40, 115.10, 89.06, 84.64, 74.00, 72.51, 39.42, 39.22, 33.79, 32.80, 32.68, 31.77.

### 1-(2-((((2S,3S,4R,5R)-5-(6-amino-9H-purin-9-yl)-3,4-dihydroxytetrahydrofuran-2-yl)methyl)thio)ethyl)-3-phenylurea (1h)

White solid 1h (171 mg, 77% yield). HRMS (ESI) m/z calcd for C19H23N7O4S [M+H]+: 446.1605; Found: 446.1606. 1H NMR (500 MHz, CD3OD) δ 8.50 (s, 1H), 8.36 (s, 1H), 7.30 (dd, J=8.7, 1.2 Hz, 2H), 7.21 (dd, J=8.7, 7.4 Hz, 2H), 6.99-6.91 (m, 1H), 6.06 (d, J=5.0 Hz, 1H), 4.73 (t, J=5.1 Hz, 1H), 4.33 (t, J=4.9 Hz, 1H), 4.24 (dt, J=6.5, 5.1 Hz, 1H), 3.36 (t, J=6.8 Hz, 2H), 3.08-2.94 (m, 2H), 2.72 (td, J=6.8, 5.3 Hz, 2H). 13C NMR (126 MHz, CD3OD) δ 156.80, 150.98, 148.63, 144.71, 142.54, 139.39, 128.41, 122.12, 119.19, 118.85, 89.08, 84.71, 73.92, 72.55, 39.10, 33.73, 32.60

### 1-(2-((((2S,3S,4R,5R)-5-(6-amino-9H-purin-9-yl)-3,4-dihydroxytetrahydrofuran-2-yl)methyl)thio)ethyl)-3-(4-fluorophenyl)urea (1l)

White solid 1i (183 mg, 79% yield). HRMS (ESI) m/z calcd for C19H22FN7O4S [M+H]+: 464.1511; Found: 464.1517. 1H NMR (500 MHz, CD3OD) δ 8.52 (s, 1H), 8.39 (s, 1H), 7.35-7.24 (m, 2H), 7.01-6.89 (m, 2H), 6.06 (d, J=5.0 Hz, 1H), 4.73 (t, J=5.1 Hz, 1H), 4.37-4.30 (m, 1H), 4.24 (dt, J=6.5, 5.2 Hz, 1H), 3.35 (t, J=6.9 Hz, 2H), 3.07-2.93 (m, 2H), 2.78-2.67 (m, 2H). 13C NMR (126 MHz, CD3OD) δ 160.19, 156.82, 150.71, 148.64, 144.25, 142.70, 135.60, 120.75, 120.69, 119.23, 114.85, 114.67, 89.12, 84.78, 73.94, 72.57, 39.11, 33.70, 32.58.

### 1-(2-((((2S,3S,4R,5R)-5-(6-amino-9H-purin-9-yl)-3,4-dihydroxytetrahydrofuran-2-yl)methyl)thio)ethyl)-3-(4-chlorophenyl)urea (1j)

White solid 1j (175 mg, 73% yield). HRMS (ESI) m/z calcd for C19H22ClN7O4S [M+H]+: 480.1215; Found: 480.1219. 1H NMR (500 MHz, CD3OD) δ 8.51 (s, 1H), 8.38 (s, 1H), 7.31 (d, J=9.0 Hz, 2H), 7.19 (d, J=9.1 Hz, 2H), 6.06 (d, J=5.0 Hz, 1H), 4.72 (t, J=5.1 Hz, 1H), 4.33 (t, J=4.9 Hz, 1H), 4.24 (dt, J=6.5, 5.1 Hz, 1H), 3.35 (t, J=6.8 Hz, 2H), 3.07-2.94 (m, 2H), 2.79-2.65 (m, 2H). 13C NMR (126 MHz, CD3OD) δ 156.47, 150.85, 148.62, 144.47, 142.59, 138.42, 128.27, 126.67, 119.91, 119.21, 89.12, 84.75, 73.95, 72.56, 39.07, 33.70, 32.55.

### 1-(2-((((2S,3S,4R,5R)-5-(6-amino-9H-purin-9-yl)-3,4-dihydroxytetrahydrofuran-2-yl)methyl)thio)ethyl)-3-(naphthalen-1-yl)urea (1k)

White solid 1k (139 mg, 56% yield). HRMS (ESI) m/z calcd for C23H25N7O4S [M+H]+: 496.1762; Found: 496.1764. 1H NMR (500 MHz, CD3OD) δ 8.48 (s, 1H), 8.28 (s, 1H), 7.95 (dd, J=6.2, 3.5 Hz, 1H), 7.82 (dd, J=6.0, 3.5 Hz, 1H), 7.62 (td, J=8.2, 1.2 Hz, 2H), 7.44 (dt, J=6.4, 3.4 Hz, 2H), 7.42-7.37 (m, 1H), 6.03 (d, J=5.0 Hz, 1H), 4.71 (t, J=5.2 Hz, 1H), 4.32 (t, J=4.9 Hz, 1H), 4.24 (td, J=5.9, 4.5 Hz, 1H), 3.41 (t, J=6.7 Hz, 2H), 3.02 (qd, J=14.0, 5.9 Hz, 2H), 2.75 (td, J=6.8, 4.8 Hz, 2H). 13C NMR (126 MHz, CD3OD) δ 157.84, 150.56, 148.49, 144.09, 142.65, 134.30, 133.72, 128.19, 127.99, 125.61, 125.33, 124.58, 121.45, 120.64, 119.12, 117.18, 89.13, 84.72, 73.92, 72.54, 39.41, 33.88, 32.70.

### 1-(2-((((2S,3S,4R,5R)-5-(6-amino-9H-purin-9-yl)-3,4-dihydroxytetrahydrofuran-2-yl)methyl)thio)ethyl)-3-ethylurea (1l)

White solid 1l (155 mg, 78% yield). HRMS (ESI) m/z calcd for C15H23N7O4S [M+H]+: 398.1605; Found: 398.1608. 1H NMR (500 MHz, DMSO-d6) δ 8.56 (s, 1H), 8.36 (s, 1H), 5.91 (d, J=5.8 Hz, 1H), 4.68 (t, J=5.4 Hz, 1H), 4.12 (dd, J=5.1, 3.7 Hz, 1H), 4.05-3.98 (m, 1H), 3.11 (t, J=7.0 Hz, 2H), 2.96 (q, J=7.2 Hz, 2H), 2.92-2.79 (m, 2H), 2.52 (td, J=6.9, 3.9 Hz, 2H), 0.94 (t, J=7.2 Hz, 3H). 13C NMR (126 MHz, DMSO-d6) δ158.24, 153.13, 149.33, 148.74, 141.91, 119.41, 88.00, 84.71, 73.46, 72.94, 34.49, 34.23, 32.96, 16.12.

### 1-(2-((((2S,3S,4R,5R)-5-(6-amino-9H-purin-9-yl)-3,4-dihydroxytetrahydrofuran-2-yl)methyl)thio)ethyl)-3-cyclohexylurea (1m)

White solid 51 (198 mg, 88% yield). HRMS (ESI) m/z calcd for C19H29N7O4S [M+H]+: 452.2075; Found: 452.2083. 1H NMR (500 MHz, CD3OD) δ 8.52 (s, 1H), 8.40 (s, 1H), 6.06 (d, J=5.1 Hz, 1H), 4.73 (t, J=5.2 Hz, 1H), 4.39-4.29 (m, 1H), 4.22 (td, J=5.9, 4.4 Hz, 1H), 3.42 (tt, J=10.6, 4.1 Hz, 1H), 3.27 (t, J=7.1 Hz, 2H), 3.05-2.89 (m, 2H), 2.65 (td, J=6.7, 3.3 Hz, 2H), 1.86-1.79 (m, 2H), 1.75-1.67 (m, 2H), 1.63-1.55 (m, 1H), 1.40-1.28 (m, 2H), 1.23-1.06 (m, 3H). 13C NMR (126 MHz, CD3OD) δ 158.92, 150.90, 148.68, 144.50, 142.62, 119.22, 89.06, 84.69, 73.94, 72.53, 48.55, 39.27, 33.72, 33.28, 32.76, 25.31, 24.67.

### Synthesis of 6

To a suspension of 2′,3′-O-isopropylideneadenosine (1.00 g, 3.25 mmol) in 1,4-dioxane (10 mL) was added DPPA (1.40 mL, 6.51 mmol) and DBU (1.46 mL, 9.77 mmol) at room temperature under N2. The solution was stirred for 2 h. Next, NaN3 (1.06 g, 16.30 mmol) and 15-crown-5 (33 mol) were added to the reaction mixture and heated to reflux. After 1 h the solid was removed by filtration. The volatiles were removed under reduced pressure to yield the crude product, purified by silica column (MeOH/DCM, 1/50). The collected fractions were dried under vacuum to offer a light-yellow solid (0.98 g, 90% yield). LRMS (ESI) m/z calcd for C13H16N8O3 [M+H]+: 333.14; Found: 333.12.

### Synthesis of 7

To a stirring solution of 6 (800 mg, 2.4 mmol) in 20 mL degassed MeOH was added 100 mg of 10% Pd/C under hydrogen. And the mixture was stirred under hydrogen overnight. The solid was removed by filtration. Next, N-Boc-2-aminoacetaldehyde (381 mg, 2.4 mmol) and sodium cyanoborohydride (181 mg, 2.88 mmol) were added to the combined filtrates. The mixture was stirred at room temperature for 4 h. After the reaction was finished, the solvent was removed under a vacuum. The generated residue was diluted with H2O (200 mL) and extracted with EtOAc (3×100 mL). The combined organic layers were washed with H2O and brine, and dried with anhydrous sodium sulfate. The volatiles were removed under reduced pressure to afford a crude oil, which was separated with a silica column (MeOH/DCM, 1/50) to generate 7 (885 mg, 82% yield). 1H NMR (500 MHz, CD3OD) δ 8.27 (s, 1H), 8.22 (s, 1H), 6.14 (d, J=3.0 Hz, 1H), 5.44 (d, J=3.1 Hz, 1H), 4.99 (dd, J=6.4, 3.4 Hz, 1H), 4.32 (td, J=6.0, 3.4 Hz, 1H), 3.11 (q, J=6.5 Hz, 2H), 2.90 (d, J=6.1 Hz, 2H), 2.72-2.59 (m, 2H), 1.58 (s, 3H), 1.36 (d, J=2.7 Hz, 13H). 13C NMR (126 MHz, MeOD) δ 157.08, 156.02, 152.66, 148.93, 140.50, 119.30, 114.31, 90.16, 85.11, 83.51, 82.44, 78.65, 50.67, 48.70, 39.23, 27.36, 26.16, 24.25. LRMS (ESI) m/z calcd for C20H31N7O5 [M+H]+ 450.25; Found: 450.26.

### Synthesis of 8

To a stirring solution of 7 (898 mg, 2.0 mmol) in 10 mL THE and 3 mL H2O was added K2CO3 (828 mg, 6.0 mmol) and Cbz chloride (510 mg, 3.0 mmol) under an ice bath. The reaction mixture was stirred overnight, slowly increasing to room temperature. After the reaction, the mixture was diluted with 200 mL H2O and extracted with EtOAc (3×100 mL). The combined organic layers were washed with H2O and brine, dried with anhydrous sodium sulfate, and concentrated under reduced pressure to afford crude oil.

The crude oil from the previous step was dissolved in 21 mL DCM and 3 mL TFA. The reaction mixture was stirred at room temperature for 1 h and concentrated under reduced pressure. The residue was washed with cold Et2O (3×50 mL) to afford a white solid 8 (725 mg, 75% yield in two steps), which was used in the next step without purification. LRMS (ESI) m/z calcd for C23H29N7O5 [M+H]+: 484.23; Found: 484.21.

### Synthesis of Compound 9

To a stirring solution of 8 (483 mg, 1.0 mmol) in 20 mL DCM was added Et3N (303 mg, 3.0 mmol) and benzyl isocyanate (133 mg, 1.0 mmol) at room temperature. The reaction mixture was stirred at room temperature for 4 h and concentrated under reduced pressure. The generated residue was diluted 150 mL H2O and extracted with EtOAc (3×75 mL). The combined organic layers were washed with H2O and brine, dried over anhydrous sodium sulfate, and concentrated under reduced pressure to afford a crude oil directly used in the next step without purification.

The crude oil from the last step was dissolved in 20 mL MeOH. Then, 10% Pd/C (106 mg, 0.1 mmol) and HCO2NH4 (252 mg, 4.0 mmol) were added to the reaction mixture and heated under reflux for 2 h. After the reaction was complete, the mixture was filtered. The filtrates were concentrated in vacuo to afford crude oil, directly used in the next step without purification.

The crude oil from the last step was dissolved in 4 mL H2O and 16 mL TFA at 0° C. The mixture was stirred for 24 h at room temperature and concentrated under reduced pressure. The generated crude oil was separated with prep-HPLC (MeCN/H2O) to afford 9 (270 mg, 61% yield in three steps). 1H NMR (500 MHz, CD3OD) δ 8.41 (s, 1H), 8.40 (s, 1H), 7.28-7.24 (m, 2H), 7.21-7.15 (m, 3H), 6.08 (d, J=5.4 Hz, 1H), 4.86-4.80 (m, 1H), 4.44-4.33 (m, 2H), 4.20-4.06 (m, 2H), 3.64 (dd, J=12.9, 10.2 Hz, 1H), 3.49-3.43 (m, 1H), 3.32-3.29 (m, 1H), 3.26-3.14 (m, 2H), 3.01 (s, 1H). 13C NMR (126 MHz, CD3OD) δ 160.41, 151.78, 148.41, 145.84, 143.08, 139.39, 128.07, 126.78, 126.67, 119.83, 90.41, 80.21, 73.47, 71.87, 49.42, 48.83, 43.33, 36.73. LRMS (ESI) m/z calcd for C20H26N8O4 [M+H]+: 443.21; Found: 443.21.

### Synthesis of Compound 10

To a stirring solution of 9 (221 mg, 0.5 mmol) in 20 mL MeOH was added tert-butyl N-(3-oxopropyl)carbamate (104 mg, 0.6 mmol) and sodium cyanoborohydride (44 mg, 0.7 mmol). The mixture was stirred at room temperature for 4 h and concentrated under reduced pressure. The generated residue was diluted with H2O (50 mL) and extracted with EtOAc (3×30 mL). The combined organic layers were washed with H2O and brine, dried over anhydrous sodium sulfate, and concentrated under reduced pressure to afford crude oil. [0151] The crude oil from the last step was dissolved in 10 mL DCM. Then 5 mL of TFA was slowly added to the mixture at room temperature, stirred for 4 h, and concentrated under reduced pressure. The generated residue was separated with prep-HPLC (MeCN/H2O) to afford 10 (202 mg, 81% yield in two steps). 1H NMR (500 MHz, CD3OD) δ 8.42 (s, 1H), 8.39 (s, 1H), 7.33-7.25 (m, 2H), 7.24-7.12 (m, 3H), 6.09 (d, J=4.9 Hz, 1H), 4.82 (t, J=5.0 Hz, 1H), 4.52 (ddd, J=10.7, 4.7, 2.5 Hz, 1H), 4.39 (t, J=4.9 Hz, 1H), 4.15-4.00 (m, 2H), 3.82 (dd, J=13.7, 10.7 Hz, 1H), 3.63 (dd, J=13.7, 2.5 Hz, 1H), 3.57-3.33 (m, 6H), 3.02 (t, J=7.4 Hz, 2H), 2.27-2.04 (m, 2H). 13C NMR (126 MHz, CD3OD) δ 160.90, 151.73, 148.45, 145.81, 142.92, 139.24, 128.14, 126.72, 126.62, 119.76, 90.52, 78.59, 73.10, 72.10, 55.76, 43.27, 36.34, 35.72, 21.64. LRMS (ESI) m/z calcd for C23H33N9O4 [M+H]+: 500.27; Found: 500.22.

### Synthesis of YD1290

To a stirring solution of 10 (50 mg, 0.1 mmol) and 1-(3-bromophenyl)-3-N-Fmoc-thiourea (68 mg, 0.15 mmol) in anhydrous DCM/DMF (5/5 mL) were added DIPEA (40 mg, 0.3 mmol) and EDC (31 mg, 0.2 mmol) at room temperature. The reaction mixture was stirred overnight at room temperature, diluted with 80 mL H2O, and extracted with EtOAc (3×40 mL). The combined organic layers were washed with H2O and brine, dried over anhydrous sodium sulfate, and concentrated underreduced pressure to afford crude oil.

The crude oil from the last step was dissolved in 5 mL DCM and 5 mL piperidine. The mixture was stirred for 2 h at room temperature and concentrated under reduced pressure. The residue was washed with Et2O (2×30 mL) and dissolved in 4 mL MeOH for prep-HPLC separation (MeCN/H2O) to afford YD1290 (30 mg, 43% yield in two steps).

1H NMR (500 MHz, CD3OD) δ 8.38 (s, 2H), 7.51-7.45 (m, 2H), 7.35 (t, =8.0 Hz, 1H), 7.31-7.26 (m, 2H), 7.24 (ddd, J=8.1, 2.1, 1.1 Hz, 1H), 7.22-7.19 (m, 1H), 7.19-7.14 (m, 2H), 6.08 (d, J=5.0 Hz, 1H), 4.83 (t, J=5.1 Hz, 1H), 4.51 (ddd, J=10.7, 4.7, 2.4 Hz, 1H), 4.39 (t, J=4.9 Hz, 1H), 4.15-4.00 (m, 2H), 3.84 (dd, J=13.7, 10.7 Hz, 1H), 3.63 (dd, J=13.7, 2.5 Hz, 1H), 3.56-3.38 (m, 5H), 3.35 (t, J=6.8 Hz, 3H), 2.19-1.97 (m, 2H). 13C NMR (126 MHz, CD3OD) δ 161.55, 161.28, 160.93, 155.49, 152.18, 148.49, 146.51, 142.65, 139.22, 136.62, 131.13, 130.05, 128.14, 127.87, 126.74, 126.63, 123.69, 122.67, 119.76, 90.49, 78.62, 73.07, 72.13, 55.77, 43.29, 38.48, 35.79, 22.85. HRMS (ESI) m/z calcd for C30H38BrN11O4 [M+H]+: 696.2364; Found: 696.2366.

### 2.0 Biochemical Characterization of the Target Compounds

### Protein Expression and Purification

PRMT1, SAHH, NTMT1, G9a, SETD7 and NNMT were expressed and purified as reported before. (lyamu et al. 2021; Al-Hamashi et al. 2021)

PRMT3 (residues 211-531) was cloned into the pET28a-MHL expression vector for protein production. The recombinant plasmid was transformed into Escherichia coli BL21(DE3). Proteins were overexpressed in Terrific Broth medium and induced with 0.15 mM IPTG at 16° C. overnight. PRMT4 (residues 140-480) was cloned into the pFBOH-MHL vector and expressed in Sf9 insect cells. After sonification, proteins were purified by Ni-NTA agarose column and eluted with the elution buffer (20 mM Tris-HCl PH8.0, 100 mM NaCl, 400 mM imidazole). Subsequently, the eluted sample flowed through a HiTrap™ Q HP column (GE Healthcare) pre-equilibrated with 20 mM Tris-HCl PH 8.0, 100 mM NaCl. The sample was then purified by Superdex 200 10/300 (GE Healthcare). The gel-filtration buffer was 20 mM Tris-HCl PH 8.0, 100 mM NaCl, 1 mM DTT. PRMT3 peak fractions were collected and concentrated to 10 mg/mL for crystallization, while PRMT4 was concentrated to 18 mg/mL for crystallization.

### Inhibition Activity of the Target Compounds

A fluorescence-based SAHH-coupled assay was applied to study the inhibition activity of all synthesized compounds on PRMT 1 and 3.

The PRMT1 methylation assay was performed under the following conditions in a final well volume of 100 μL: 2.5 mM HEPES (pH=7.0), 25 mM NaCl, 25 μM EDTA, 50 μM TCEP, 0.01% Triton X-100, 5 μM SAHH, 0.1 μM PRMT1, 10 μM AdoMet, and 10 μM ThioGlo4. After incubating with the inhibitors for 10 min at 37° C., reactions were initiated by the addition of 5 μM H4-21 peptide (Km value). The fluorescence signal was monitored on a BMG CLARIOstar microplate reader with excitation at 400 nm and emission at 465 nm for 15 min. Data were processed using GraphPad Prism software 8.0.

The PRMT3 methylation assay was performed under the following conditions in a final well volume of 100 μL: 20 mM Tris (pH=7.5), 0.01% Triton X-100, 5 μM SAHH, 0.25 μM PRMT3, 30 μM AdoMet, and 15 μM ThioGlo4. After incubating with the inhibitors for 10 min at 30° C., reactions were initiated by the addition of 30 μM H4-21 peptide (Km value). Fluorescence was monitored on a BMG CLARIOstar microplate reader with excitation 380 nm and emission 505 nm. Data were processed by using GraphPad Prism software 8.0. All experiments were performed in duplicate.

### Inhibition Mechanism Study

The inhibition mechanism of YD1113 on PRMT3 was studied with the fluorescence-based SAHH-coupled assay. Varying concentrations of SAM (from 30 to 240 μM) with 30 μM fixed concentration of H4-21 or varying concentration of H4-21 (from 15 to 240 μM) with 30 μM fixed concentration of SAM were included in reactions at concentration of YD1113 ranging from 1.5 nM to 100 μM.

The inhibition mechanism of YD1290 was study on PRMT1 with the fluorescence-based SAHH-coupled assay. Varying concentrations of SAM (from 5 to 40 μM) with 4 μM fixed concentration of H4-21 or varying concentration of H4-21 (from 4 to 32 μM) with 5 μM fixed concentration of SAM were included in reactions at concentration of YD1290 ranging from 0.15 nM to 10 μM.

All the IC₅₀ values were determined in triplicate. Fluorescence was monitored on a BMG CLARIOstar microplate reader with excitation 380 nm and emission 505 nm. Data were processed by using GraphPad Prism software 8.0.

### Selectivity

A fluorescence-based SAHH-coupled assay was applied to study the effect of the compound on methyltransferase activity of NTMT1, SETD7, G9a, NNMT, tbPRMT7 and SAHH. For NTMT1, the assay was performed in a final well volume of 100 μL: 25 mM Tris (pH=7.5), 50 mM KCl, 0.01% Triton X-100, 5 μM.

SAHH, 0.1 μM NTMT1, 3 μM AdoMet, and 10 μM ThioGlo4. After incubation for 10 min with the inhibitor, reactions were initiated by the addition of 0.5 μM GPKRIA peptide, and the reaction was monitored for 15 min. For SETD7, the assay was performed in a final well volume of 100 μL: 25 mM potassium phosphate buffer (pH=7.6), 0.01% Triton X-100, 5 μM SAHH, 1 μM SETD7, 2 μM AdoMet, and 10 μM ThioGlo1. After incubation for 10 min with the inhibitor, reactions were initiated by the addition of 90 μM H3-21 peptide, and the reaction was monitored for 15 min. For G9a, the assay was performed in a final well volume of 100 μL: 25 mM potassium phosphate buffer (pH=7.6), 1 mM EDTA, 2 mM MgCl2, 0.01% Triton X-100, 5 μM SAHH, 0.1 μM His-G9a, 10 μM AdoMet, and 10 μM ThioGlo4. For NNMT, the assay was performed in a final well volume of 100 μL: 25 mM Tris (pH=7.5), 50 mM KCl, 0.01% Triton X-100, 5 μM SAHH, 0.1 μMNNMT, 10 μM AdoMet, and 10 μM ThioGlo1. After incubation for 10 min with the inhibitor, reactions were initiated by the addition of 10 μM nicotinamide, and the reaction was monitored for 18 min. For tbPRMT7, the assay was performed in a final well volume of 100 μL: 25 mM Tris (pH=7.5), 50 mM KCl, 0.01% Triton X-100, 5 μM SAHH, 0.2 μM PRMT7, 3 μM AdoMet, and 15 μM ThioGlo1. After incubation for 10 min with the inhibitor, reactions were initiated by the addition of 60 μM H4-21 peptide, and the reaction was monitored for 15 min. For The inhibitors were added at three concentrations: 100, 33.3, 11.1 μM.

The effect of the inhibitors on the coupled enzyme, SAHH, was also evaluated. The assay was performed in a final well volume of 100 μL: 25 mM Tris (pH=7.5), 50 mM KCl, 0.01% Triton X-100, 0.1 μM SAHH, and 15 μM ThioGlo1. After incubation for 10 min with the compound, 0.5 μM SAH was added to initiate the reactions.

All experiments were performed in duplicate. Fluorescence was monitored on a BMG CLARIOstar microplate reader with excitation at 380 nm and emission at 505 nm.

The selectivity of compounds YD1113 and YD1290 on PRMTs were performed by Reaction Biology Corp.

### Thermal Shift Assay

The fluorescence-based thermal shift assay was performed using a StepOne plus qPCR instrument (Thermal Fisher). For PRMT1, 10 μM of protein was mixed with 2× sypro orange and DMSO, or 100 μM SAM, or 100 μM YD1113, or 100 μM YD1290 in a buffer consisting of 2.5 mM HEPES (pH=7.0), 25 mM NaCl, 25 μM EDTA, 50 μM TCEP, 0.01% Triton X-100. For PRMT3, 20 μM of protein was mixed with 2× sypro orange and DMSO, or 100 μM SAM, or 100 μM YD1113, or 100 μM YD1290 in a buffer consisting of 20 mM Tris (pH=7.5), 0.01% Triton X-100. For PRMT4, 20 μM of protein was mixed with 2× sypro orange and DMSO, or 100 μM SAM, or 100 μM YD1113, or 100 μM YD1290 in a buffer consisting of 20 mM bicine (pH=8.5), 0.01% Triton X-100. All samples were heated from 25° C. to 75° C. at an increased rate of 0.5° C. per minute. Protein denaturation was monitored by the increased fluorescence signal of Sypro Orange, which captures exposed hydrophobic residues during thermal unfolding. The recorded curves were analyzed by the software StepOne plus and plotted in GraphPad Prism 8 software.

### Co-Crystallization and Structure Determination

Proteins were incubated with compounds at a 1:2 molar ratio for 0.5 h on ice. Afterwards, protein-compound complexes were crystallized by sitting drop vapor diffusion method with mixing 1 μl proteins and 1 μl reservoir solutions. The crystals of PRMT3-II710 were grown in 20% PEG3350, 0.3 M Sodium Formate. The crystals of PRMT3-YD1113 were obtained in 0.1 M Tris·HCl (pH 8.5), 2.0 M Ammonium sulfate. The PRMT4-YD1113 was crystallized in 0.2 M Calcium chloride dihydrate, 0.05 M HEPES sodium pH 7.5, 28% v/v Polyethylene glycol 400, 0.002 M Spermine. The PRMT4-YD1290 was crystallized in 0.1 M BICINE pH 9.0, 2.0 M Magnesium chloride hexahydrate. The crystals were flash-frozen in liquid nitrogen using a cryoprotetant consisting for reservoir solution supplemented with 15% glycerol.

X-ray diffraction data for both PRMT3+YD1113, II710 and PRMT4+YD1290, YD1113 were collected at 100K on the 241D-E or 241D-C of NCAT at Advanced Photon Source (APS), Argonne National Laboratory and all the data was processed using the HKL-3000 suite. (Otwinowski & Minor 1997)

All 4 structures were solved by molecular replacement using PHASER with PDB entry 2FYT and 5U4X as search template for PRMT3 and PRMT4 respectively. REFMAC was used for all structure refinement. (McCoy et al. 2007; Murshudov et al. 1997) Geometry restraints for all compound refinement were prepared with by GRADE developed at Global Phasing Ltd. (Smart et al. 2012) Graphics program COOT was used for all model building and visualization. (Emsley et al. 2004) MOLPROBITY was used for structure validation. (Davis et al. 2004)

Structural Alignment of PRMT3 with Other PRMTs

Structural alignment of PRMT3 with other PRMTs were taken with the protein structure alignment module in Maestro 12.8. The structures and interaction diagram were drawn with Maestro. PRMT1 (PDB ID, 6NT2), PRMT4 (PDB ID, 51H3), PRMT5 (PDB ID, 4X63), PRMT6 (PDB ID, 4Y2H), PRMT7 (PDB ID, 4C4A) and PRMT8 (PDB ID, 4X41) were obtained from protein data bank (www.rcsb.org).

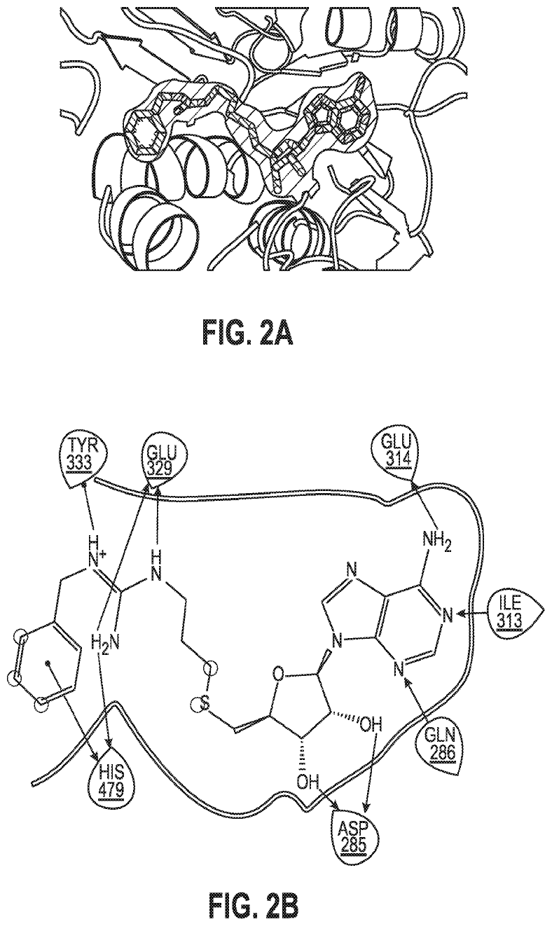

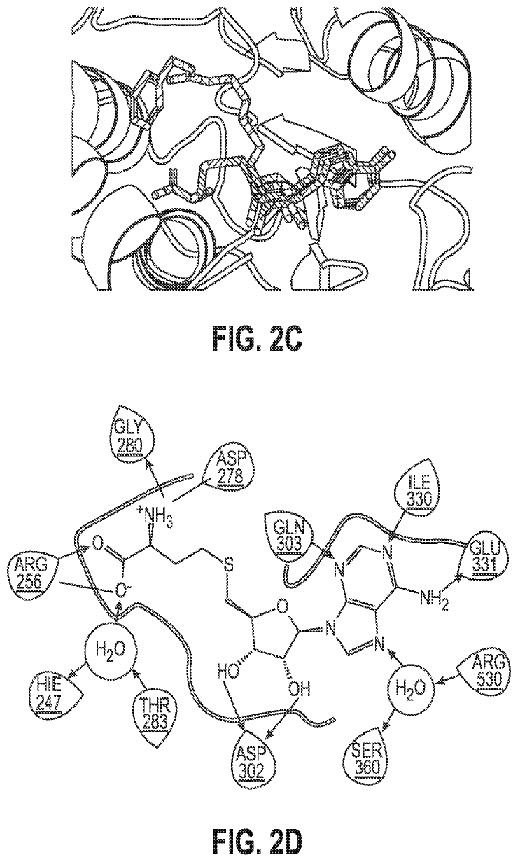

To dissect the binding mode of PRMT inhibitors that connect 5′-thioadenosine with a tripeptide through a substituted guanidine group the co-crystal structures of PRMT3 complexed with II710 (1a, IC₅₀=12 μM for PRMT3) (FIG. 2) was obtained. The overall topology of the PRMT3-II710 complex aligned well with the reported co-crystal structure of the PRMT3-SAH (PDB ID: 2FYT), which clearly showed II710 as a bisubstrate analogue residing in the active site of PRMT3 (FIG. 2a). Specifically, the thioadenosine moiety of II710 aligned well with the adenosine moiety of SAH, forming hydrogen bonds with Asp 285, Gin 286, Ile 313, and Glu 314. The benzyl guanidine group protrudes into the arginine binding channel to interact with Glu 329, Tyr 333, and His 479 (FIG. 2b), confirming the bisubstrate feature of inhibition. Compared to SAH, II710 had no interaction with the homocysteine binding site (FIG. 2c-2d).

To dissect the binding mode, the co-crystal structures of PRMT3 complexed with II710 (1a, IC₅₀=12 μM for PRMT3) (FIG. 2) was obtained. The overall topology of the PRMT3-II710 complex aligned well with the reported cocrystal structure of the PRMT3-SAH (PDB ID: 2FYT), which clearly showed II710 as a bisubstrate analogue residing in the active site of PRMT3 (FIG. 2a). Specifically, the thioadenosine moiety of II710 aligned well with the adenosine moiety of SAH, forming hydrogen bonds with Asp 285, Gln 286, Ile 313, and Glu 314. The benzyl guanidine group protrudes into the arginine binding channel to interact with Glu 329, Tyr 333, and His 479 (FIG. 2b), confirming the bisubstrate feature of inhibition. Compared to SAH, II710 had no interaction with the homocysteine binding site (FIG. 2c-2d).

To enhance the inhibition, the guanidino group was replaced with a urea group to afford 1b, inspired by the PRMT 5/7 dual inhibitor DS437 bearing a 2-C atom linker between the 5′-thioadenosine and urea group.13 However, 1b only inhibited 50% of PRMT3 activity at 70 μM. Given the importance of the linker length for the bisubstrate analogues,14, 15 the linker was reduced from a 3C- to 2C-atom to produce YD1113 (1c), resulting in 140-fold increased inhibition for PRMT3. However, a similar modification in II710 generated 1d containing a 2C-atom linker, causing a 3-fold decrease. Next, the effect of substitutions on the urea moiety of YD1113 (Table 1) was examined. Briefly, introducing a 4-fluoro (1e) group to the benzyl group of YD1113 led to an over 10-fold reduction. Replacement of the benzyl group with phenylethyl (1f) or phenylpropyl (1g) abolished the inhibition activity (>100 μM). Whereas replacing the benzyl ring of YD1113 with phenyl (1h) or parafluorophenyl ring (1i) only led to a 4-fold decreased inhibition, a para-chlorophenyl (1j) reduced 10-fold compared to 1i with a fluoride substitution. Not surprisingly, naphthalene (1k) caused over 40-fold reduction compared to phenyl (1h). The decreased activity of 1c to 1g and 1 h to 1k indicated that the binding site of the benzyl urea group is sensitive to the steric effects, implying a narrow space. However, 11 and 1m with ethyl and cyclohexane groups showed IC₅₀ values over 100 μM, suggesting the benzyl ring of YD1113 may contribute to the binding by forming pi-pi or cation-pi interactions.

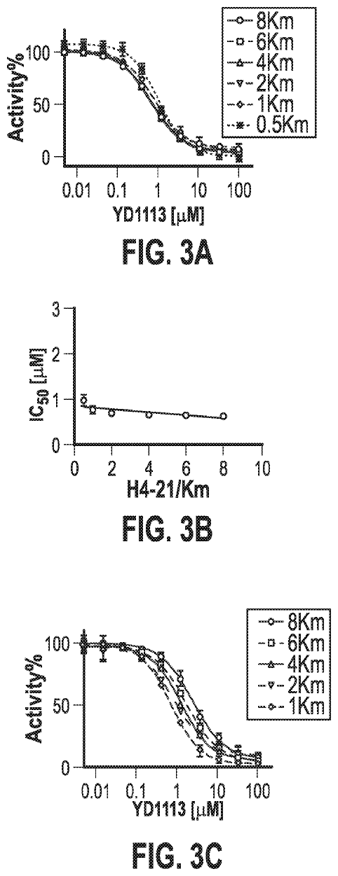

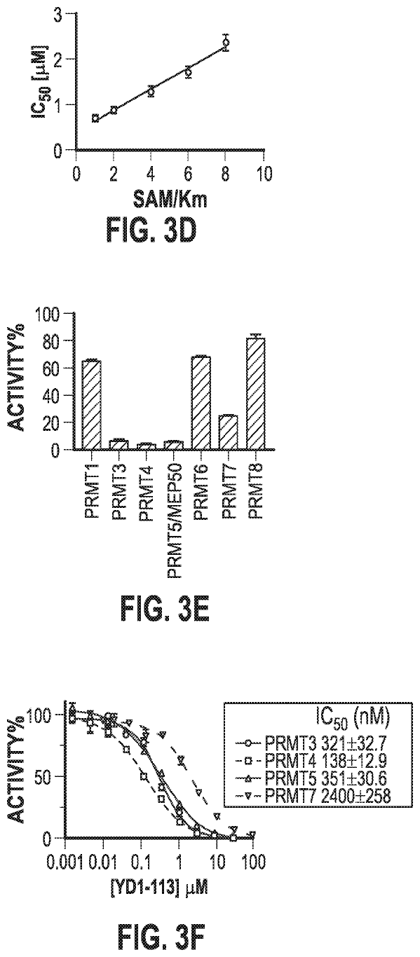

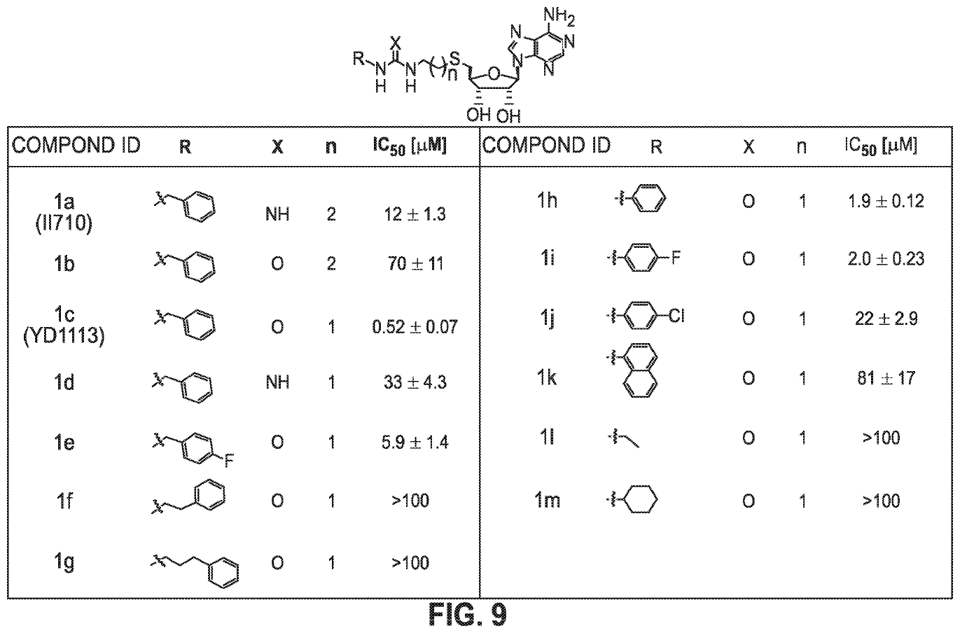

The sensitivity of YD1113 to the steric effects in the above SAR studies (Table 1; FIG. 9) is inexplicable by a relatively open and tolerable peptide substrate binding channel of PRMTs. (Tewary et al. 2019) This contradiction prompted the hypothesis that YD1113 binds to an alternative pocket rather than acts as a bisubstrate analogue to reach the peptide binding pocket. This hypothesis was tested by investigating its inhibition mechanism with a SAHH-coupled fluorescence-based assay. (Richardson et al. 2015) The IC₅₀ values of YD1113 remained constant with the increased ratio of peptide/Km (FIG. 3a-b) while increasing linearly with the ratio of SAM/Km (FIG. 3c-d). This result demonstrated that YD1113 is competitive to SAM and noncompetitive to the peptide substrate of PRMT3, supporting the hypothesis. Thus, the urea substitution altered the binding mode of YD1113 compared to the guanidine analogue II710 with the bisubstrate feature, as YD1113 lost engagement with the peptide binding site.

Next, the selectivity of YD1113 was investigated on a panel of PRMTs at 10 μM. YD1113 showed significant inhibition to PRMT3, 4, and 5 (>95%), moderate inhibition to PRMT7 (>75%), and limited inhibition to PRMT1, 6, and 8 (<35%) (FIG. 3e). As YD1113 exhibited over 50% inhibition for PRMT3, 4, 5, and 7 at 10 μM, a choice was made to determine the IC₅₀ values of YD1113 for these four PRMTs. Indeed, YD1113 demonstrated a potent and comparable activity for PRMT3 and 5 with an IC₅₀ value at 321 nM and 351 nM, respectively. Moreover, YD1113 is 2-fold more potent for PRMT4 and 7-fold less potent for PRMT7 than PRMT3, respectively.

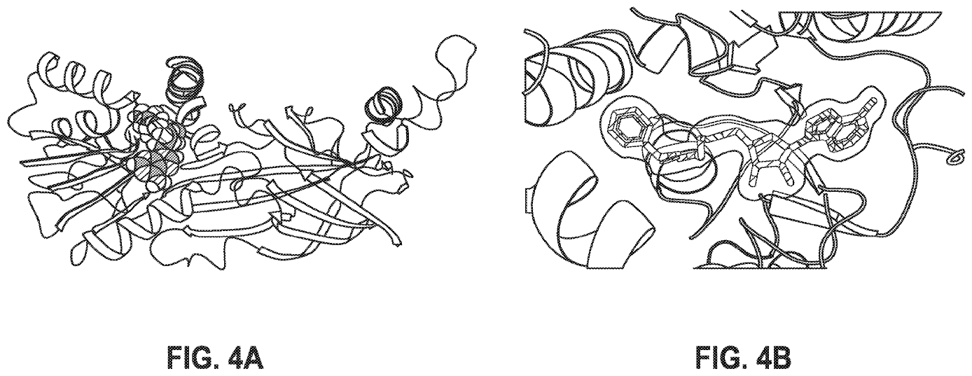

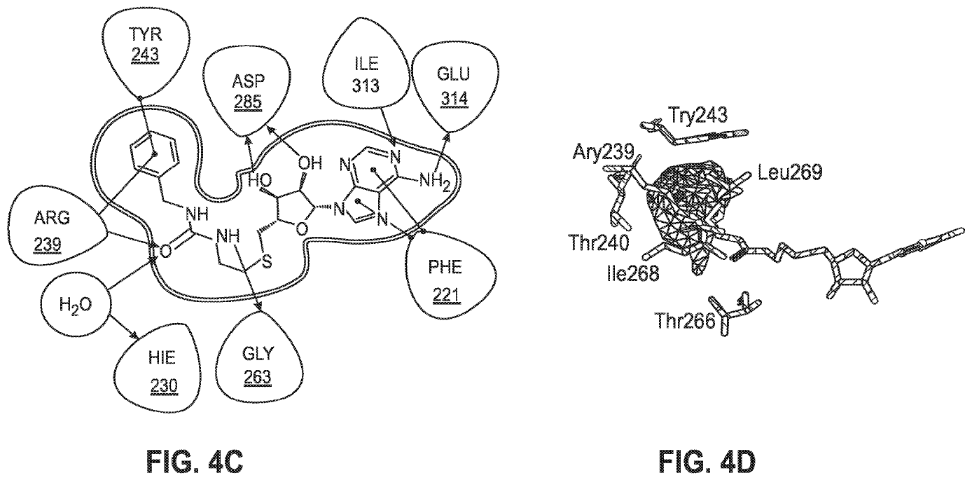

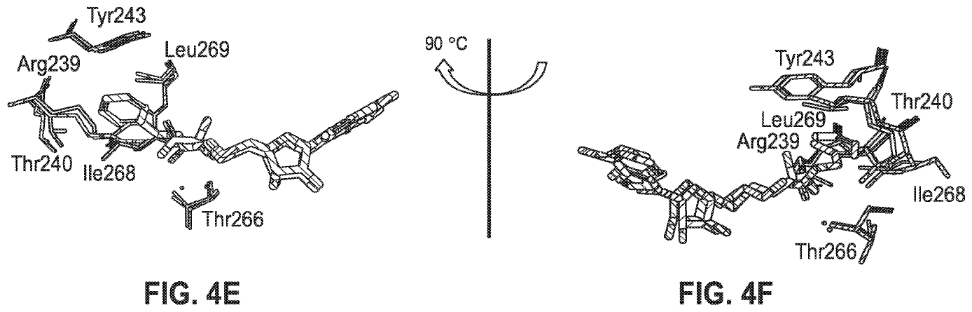

To understand the binding modes of YD1113, the X-ray structures of PRMT3 and PRMT4 complexed with YD1113, respectively, were determined. The overall topology of the PRMT3-YD1113 complex is similar to those in the complex with SAH (FIG. 4). As illustrated in the electron density map, YD1113 only occupied the SAH binding site without interactions with the double E loop region located at the edge of the substrate peptide binding channel (FIG. 4a). Similar to SAH, theadenosine moiety of YD1113 interacted with Phe221, Asp285, Ile313, and Glu314 (FIG. 4b). The urea group bound at the same position as the α-amino carboxylate of SAH, forming similar interactions with Arg 239, Gly 263, and water (FIG. 4b-4c). Strikingly, the benzyl urea reached into a hitherto unrecognized pocket adjacent to the binding pocket of a-amino carboxylate moiety of SAH (FIG. 4). Remarkably, the conformations of Arg 239, Thr 240, Ile 268, and Leu 269 were altered to create a hydrophobic pocket (FIG. 4d), where Arg 239, Ile 268, and Leu 269 located at the entrance and stacked with the benzyl group in a sandwich manner. Meanwhile, Tyr 243 at the bottom of the hydrophobic pocket formed a pi-pi interaction with the benzyl group. Furthermore, limited space in this new pocket provided a structural basis to explain why different substitutions on the benzyl ring of YD1113 substantially impacted its inhibitory activity on PRMT3 (Table 1). For example, the SARs studies supported that the pi-pi interaction between Tyr 243 and the benzyl group was critical for binding, as exemplified by a dramatic decrease in binding with a cyclohexane or ethyl replacement. In summary, the co-crystal structure provided the first evidence that the benzyl urea functioned as a unique mimic for the a-amino carboxylate moiety of SAH for binding to PRMT3. This newly uncovered pocket does not exist in the previous structures of PRMT3 in complexes with SAH or II710. Thus, this uniquely induced binding pocket was attributed to the benzyl urea of YD1113.

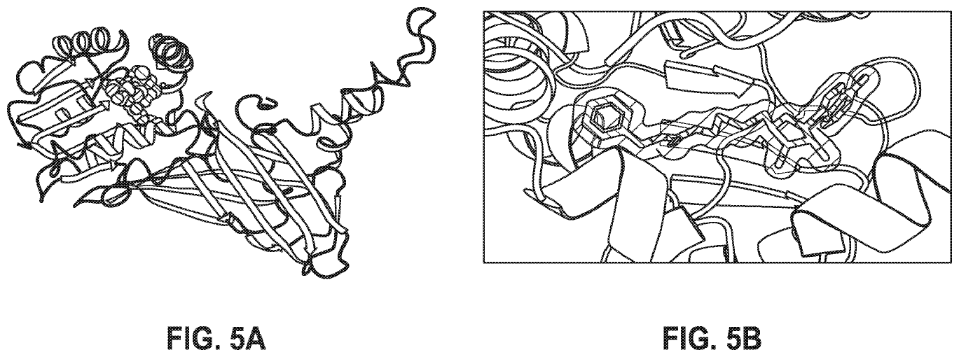

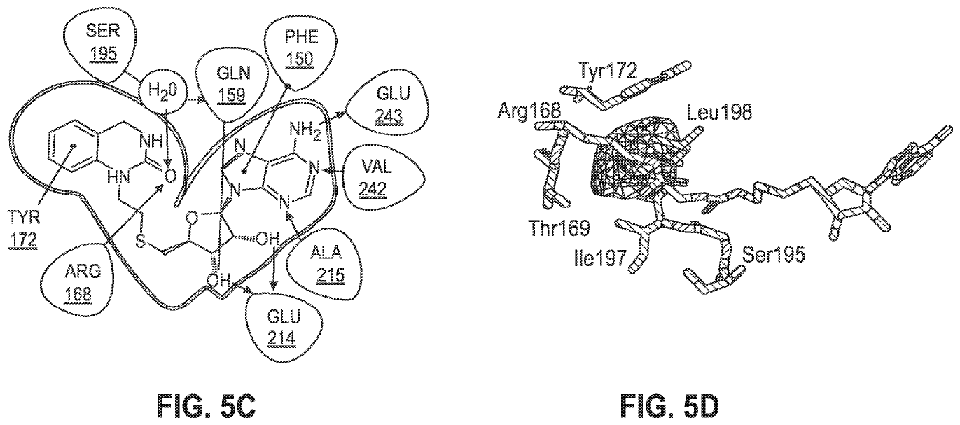

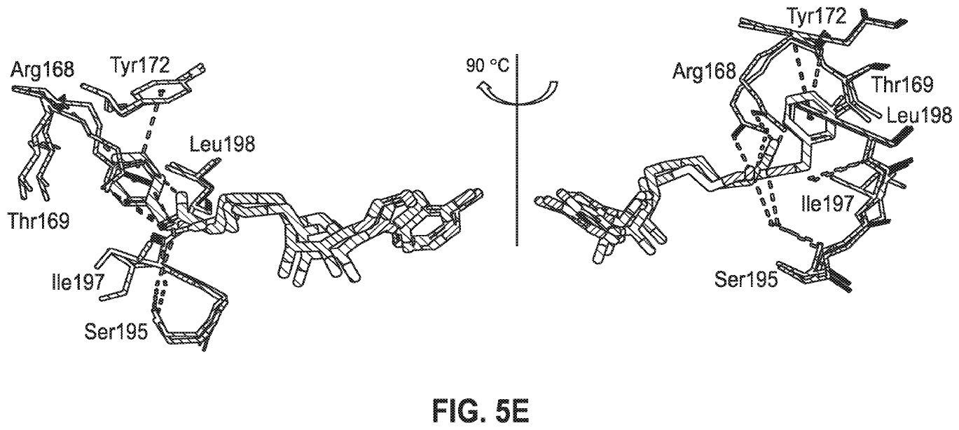

Likewise, YD1113 displayed a similar binding pose in the active site of PRMT4. Briefly, the adenosine moiety of YD1113 forms hydrogen bonds with Gin 159, Glu 214, Ala 215, Val 242, and Glu 243, and pi-pi interactions with Phe 150 (FIG. 5a-5c). Meanwhile, the urea group of YD1113 reaches the location where the carboxylic acid moiety of SAH resides, forming hydrogen bonds with Arg 168 and water. In addition, the benzyl group of YD1113 protruded into a new hydrophobic pocket (formed by Arg 168, Ile 197, and Leu 198) and created a pi-pi interaction with Tyr 172 (FIG. 5b-5d). This result further certifies that YD1113 acted as a unique surrogate of SAH by interacting with both the SAM binding pocket and a newly formed hydrophobic pocket.

As the key residues of the SAM binding pocket are conserved across all PRMT members,16 it was speculated that a similar hydrophobic pocket may exist in all PRMTs. To investigate this prospect, the SAH binding pockets of all seven PRMTs with reported co-crystal structures in complex with SAH were aligned. Then the residues around the hydrophobic pocket as PRMT3 for comparison were extracted. Indeed, all type I PRMTs share identical residues (one Arg, two lie, and one Tyr) at the same position (Table S1). Interestingly, PRMT7 has similar residues (Arg 44, Leu 77, Leu 78, and Tyr 48) at the same position as type I PRMTs. Compared to type I and III PRMTs, PRMT5 has similar residues despite minor differences. For instance, PRMT5 has a Tyr 337 for the pi-pi interaction and a matching set of three residues with similar characteristics (Lys 333, Pro 370, and Leu 371) to create a hydrophobic pocket. Although no X-ray structures have been obtained for YD1113 in complex with PRMT5/7, potent inhibition of YD1113 for PRMT5/7 (IC₅₀<2.4 μM) inferred that the benzyl group may induce and interact with a hydrophobic pocket as it was observed in PRMT3/4. Altogether, it is reasonable to predict that a similar hydrophobic pocket adjacent to homocysteine commonly existed in all PRMTs, which the benzyl urea of YD1113 or similar analogues may induce. Even though the hydrophobic pocket exists in all PRMTs, it does not mean these pockets are identical, as YD1113 exhibited different inhibitory activity for each PRMT. In other words, the benzyl urea group may be fine-tuned further to increase the selectivity.

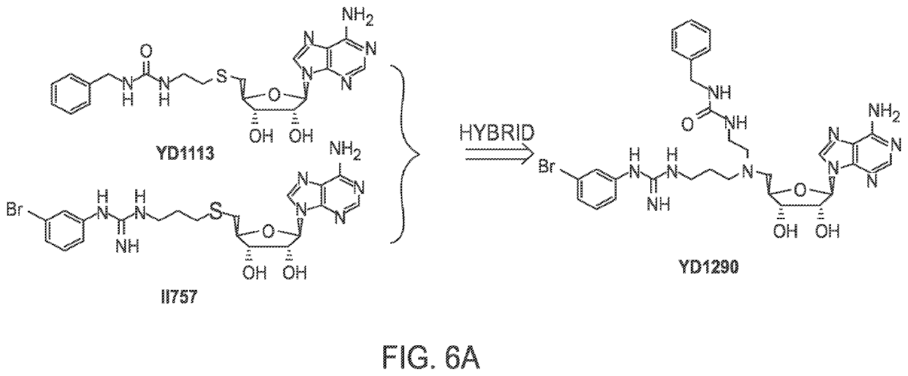

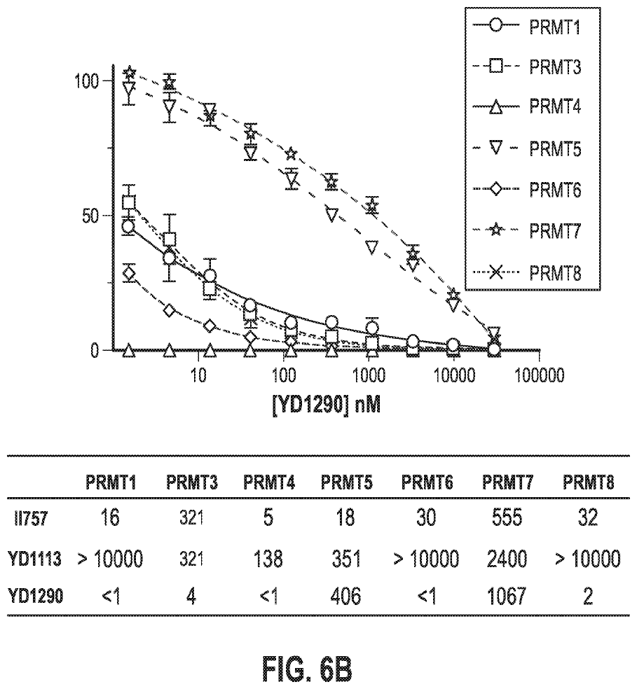

After validating the benzyl urea moiety as an unconventional homocysteine mimic of SAH for PRMTs, it was hypothesized that incorporating the benzyl urea moiety into the original pan PRMT inhibitor II757 would build a potent bisubstrate inhibitor. Hence, YD1290 was designed to merge YD1113 and II757 to form a “T-shaped” (FIG. 6a). To enable a stable tri-substitution, the sulfur atom with a nitrogen atom was replaced. YD1290 was synthesized following the reported methods 9, 15 and tested in a radioactivity assay for PRMTs (FIG. 6b). Like its parent compound II757, YD1290 demonstrated potency to all tested PRMTs. Compared to YD1113, YD1290 completely abolished PRMT4 activity even at 1.5 nM and displayed >80-fold potency to PRMT 1, 3, 4, 6, and 8. To date, YD1290 is the most potent inhibitor for type I PRMTs.

Next, the thermal shift assay (TSA) was conducted as an orthogonal method to examine the effects of YD1290 on the thermal stability of PRMT1, 3, and 4 (Figure S1). Incubation of YD1290 with all three PRMTs induced a higher thermal stabilization (ΔTm=+8.7° C., +23.4° C., and +10.9° C.) than the additive contributions of SAM (ΔTm=+1.6° C., +1.1° C., +1.9° C.) and YD1113 (ΔTm=+0.6° C., +5.8° C., +2.6° C.). These results suggested that YD1290 can significantly increase the thermal stability of the tested PRMTs, validating its interaction with PRMT1/3/4.

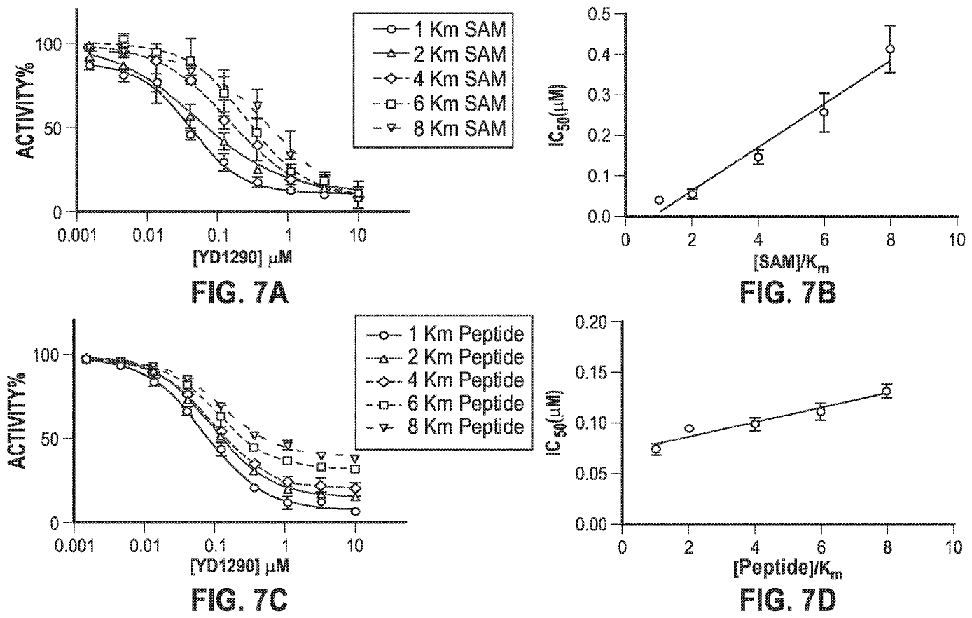

To confirm the potent inhibition activities of YD1290 on type I PRMTs resulting from bisubstrate characteristics, its inhibition mechanism on PRMT1 was determined through the SAHH-coupled fluorescence-based assay. Like II757 and YD1113, the IC₅₀ values of YD1290 increased linearly with the ratio of SAM/Km (FIG. 7a-7b), indicating YD1290 is a SAM-competitive inhibitor. However, the IC₅₀ of YD1290 was slightly increased when the ratio of peptide/Km increased (FIG. 7c-7d). These results demonstrated that YD1290 not only retained the SAM competitive ability as its parent compounds YD1113 and II757, but also gained some interactions with the substrate peptide binding pocket as II757, as it was hypothesized.

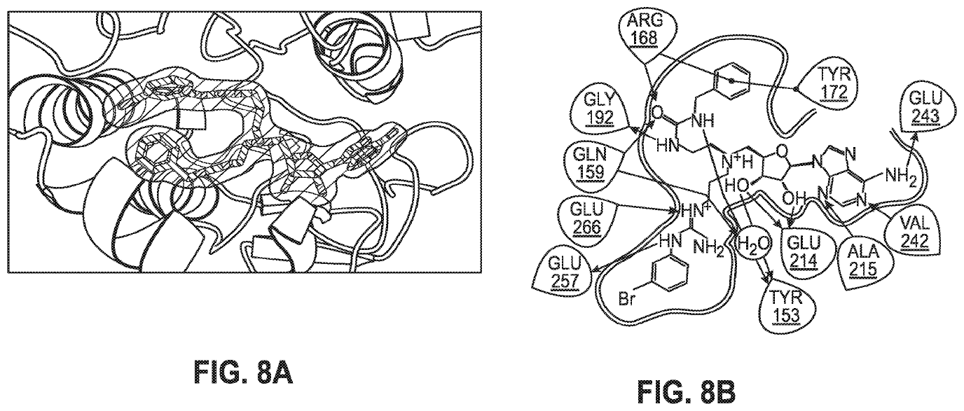

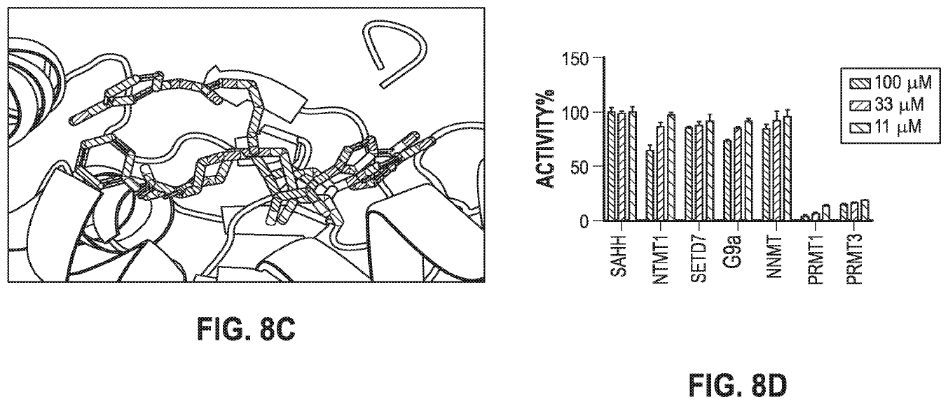

To further validate the binding mode of YD1290, the cocrystal structure of PRMT4 complexed with YD1290 was determined. Indeed, YD1290 demonstrated a “T-shaped” binding mode in the active site by engaging with both SAM and peptide binding pockets (FIG. 8a). Like YD1113, the benzyl urea moiety of YD1290 imitated the homocysteine to interact with Gin 159, Arg 168, Tyr 172, and Gly 192 (FIG. 8b-8c). Furthermore, the meta-bromobenzene guanidine of YD1290 located at the double E loop region interacted with Glu 257 and Glu 266, supporting the peptide substrate competitive ability of YD1290. In addition, the tertiary amine of YD1290 also formed a cation-pi interaction with Tyr 153.

To examine the selectivity, the inhibition of YD1290 on four inhouse methyltransferases, including protein N-terminal methyltransferase 1 (NTMT1), nicotinamide N-methyltransferase (NNMT), and two protein lysine methyltransferases PKMT (G9a and SETD7) were tested. In addition, SAHH was included as it is used in the coupled fluorescence assay and possesses a SAH binding site. As shown in FIG. 8d, YD1290 did not display any significant inhibition to SAHH, NTMT1, SETD7, G9a, and NNMT at 100 μM.

In summary, it was discovered and thoroughly verified that the benzyl urea moiety can function as an unconventional analogue of a-amino carboxylate moiety of SAH to retain the interaction with PRMTs. Preliminary SAR data also indicates that the inhibition is sensitive to the substitution in the urea group. Among them, 2-(3-benzylureido)ethyl)thioadenosine (YD1113) is a noncanonical SAH mimic to interact with PRMTs. Co-crystal structures of YD1113 complexed with PRMT3 or PRMT4 illustrated the molecular interaction, providing the first evidence to confirm the biocompatibility of 3-ethyl-1-benzyl urea to mimic homocysteine. Data boost the credibility of the projection of the urea group of DS437 to engage the homocysteine binding site.

Furthermore, it was demonstrated that the benzyl urea moiety can be applied in developing “T-shaped” PRMT bisubstrate inhibitors to engage both SAM and peptide binding pockets. YD1290 is a potent and selective type I PRMT inhibitor, implying that the active site of type II PRMTs differs from those of type I PRMTs. Compared to SAH, YD1113 is less polar with the benzyl urea to replace the a-amino carboxylate moiety. Thus, YD1113 has the potential to serve as a building block to develop potent and selective inhibitors for PRMTs.

A similar hydrophobic pocket was created in DOT1L by the tert-butyl phenyl urea moiety of EPZ004777. (Basavapathruni et al. 2012; Yu et al. 2012) It is believed that the high selectivity of EPZ004777 for DOT1L was attributed to its interactions with the hydrophobic pocket. PRMTs and DOT1L belong to the class I methyltransferases of a Rossmann fold. Because the class I Rossmann fold methyltransferases have the conserved SAM binding sites, it was speculated that a similar hydrophobic pocket may also exist in other class I methyltransferases. (Konc et al. 2010 (1); Konc et al. 2010 (2)) Meanwhile, the structural similarities between YD1113 and EPZ004777 suggest that benzyl or phenyl urea are potential pharmacophores to explore the hydrophobic pocket, and may also be adapted and tuned to discover potent inhibitors for other class I Rossmann fold methyltransferases.

### PMRT Inhibitors as Potential Drugs.

The development and mechanistic characterization of PRMT small molecule inhibitors disclosed herein provides for novel therapeutics for treating various disease states involving the misregulation and aberrant expression of PRMTs. Such diseases include and are not limited to, cancer, autoimmune disease, developmental disorders, and neurodegenerative disease.

### Design of Focused Library for Targeting PRMTs

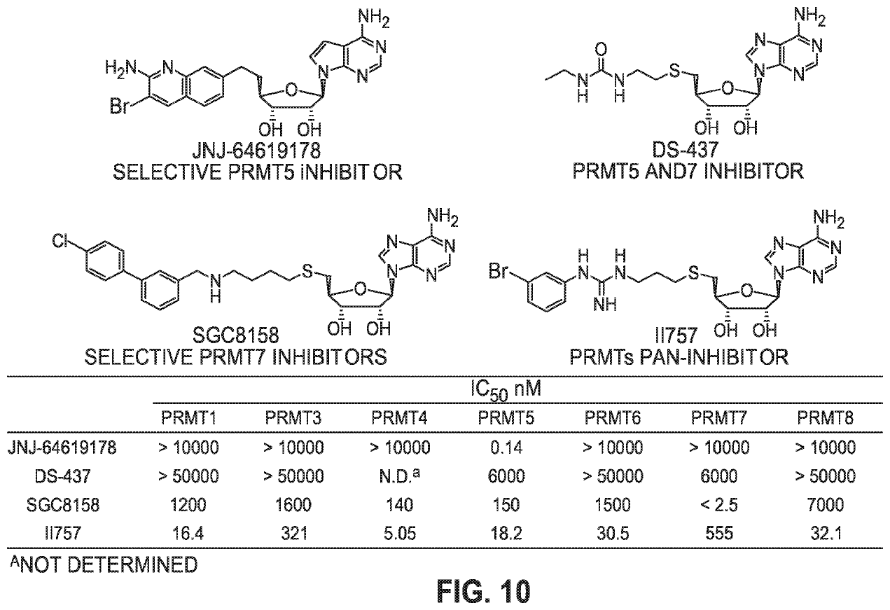

The PRMT bisubstrate inhibitor AH237 of the instant disclosure, incorporating 5′-thioadenosine and a GAR consensus motif with a propyl linker, exhibited different inhibition among PRMTs. Thus, it is rational to believe that fine difference exists among the active site of PRMTs. In addition, there are several analogues containing the core structure of adenosine, establishing the feasibility of high potency and good selectivity as PRMT inhibitors (FIG. 10). In addition to PRMT5 inhibitor JNJ-64619178, DS-437 shows dual inhibition to PRMT 5/7, and SGC8158 selectively inhibits PRMT7 inhibitor. (Szewczyk et al 2020) Recently, II757 showed broad and potent inhibition to all PRMTs. (lyamu et al 2021) Thus, thoroughly examining the active site of PRMTs would shed some light on the difference and guide us to design selective inhibitors for each PRMT. Herein, the construction of a focused SAH library to profile the active site of type I PRMTs was described.

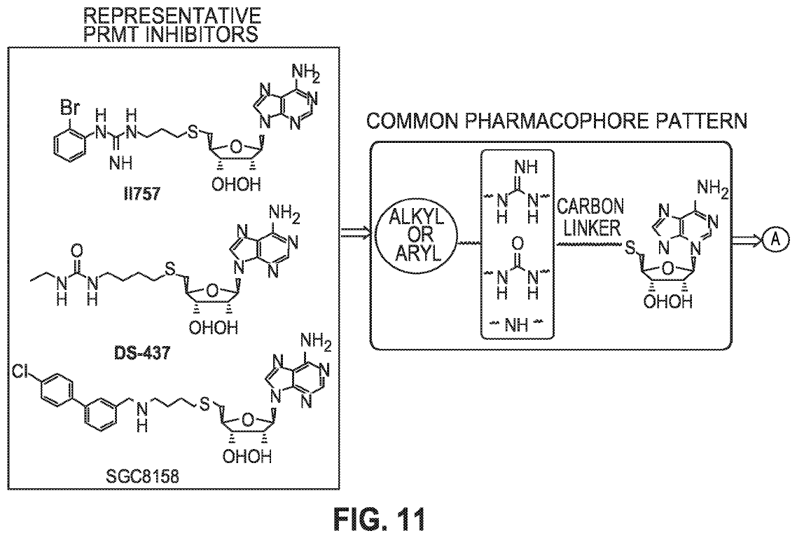

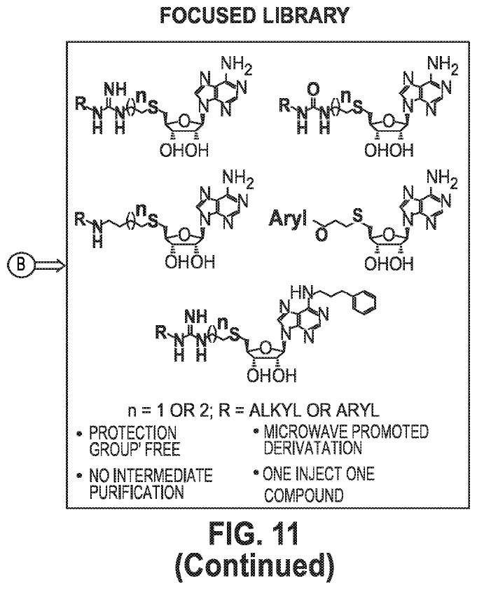

Three representative types of reported adenosine-based PRMTs inhibitors have a shared pharmacophore pattern (FIG. 11). The right part is 5′-thioadenosine. The middle part comprises a flexible carbon linker and a functional group resembling the guanidino group of the arginine side chain. The carbon linker ranges from 2C- to 4C-atom; the functional group is guanidino, urea, or secondary amine. The left part introduces different substitutions on the functional group. Despite the same 5′-thioadenosine at the right, the middle and left parts impact the selectivity and potency. Based on this pharmacophore pattern, five sets were designed to increase the diversity. The first set is II757 analogues comprising guanidino groups and different carbon linkers. In the second set, a urea group was introduce to replace the guanidino group to remove the hydrogen donation ability. The third set has a benzylamine or arylamine, with only one site hydrogen bond donor/acceptor ability. The fourth set bears a phenol ether group without the hydrogen bond donor ability. In the fifth set, a phenylpropane group at the N-6 position of adenosine was introduced to explore the steric effects in the adenine binding pocket. Altogether, a focused library containing 100 adenosine analogues was designed, which covers the carbon linker, functional groups from guanidine, urea, secondary amine to oxygen, and N-6 position of adenosine with or without substitution.

### Synthesis

The description provided here relates to a general synthetic method to efficiently construct a focused adenosine library, which would facilitate us to profile PRMTs and share it among the research community. To minimize the synthetic steps and simplify the purification procedure, a consecutive 5-step and protection-free reaction from adenosine to the final crude product was devised. Thus, only one purification was required to get the final products (Scheme 1). Briefly, the commercially available adenosine was subjected to the Mitsunob reaction to produce crude 1. The reaction mixture was first diluted with water and washed with 30% ethyl acetate in hexanes to remove triphenylphosphine oxide. Because the starting material adenosine is more polar than 1 and has a much lower solubility in ethyl acetate, the mixture was then extracted with ethyl acetate to obtain 1 in high purity for direct use. In the second step, 20% mol of triphenylphosphine was added to prevent the formation of a disulfide bond. After the substitution to install different linkers, the reaction mixture was diluted with water, neutralized with acetic acid, and washed with 30% ethyl acetate in hexane to remove any excess reactants and the majority of triphenylphosphine oxide. Next, removing the Boc group with TFA and washing the concentrated residue with diethyl ether yielded 3a-b in high purity, which proceeded to further modifications on the left part.

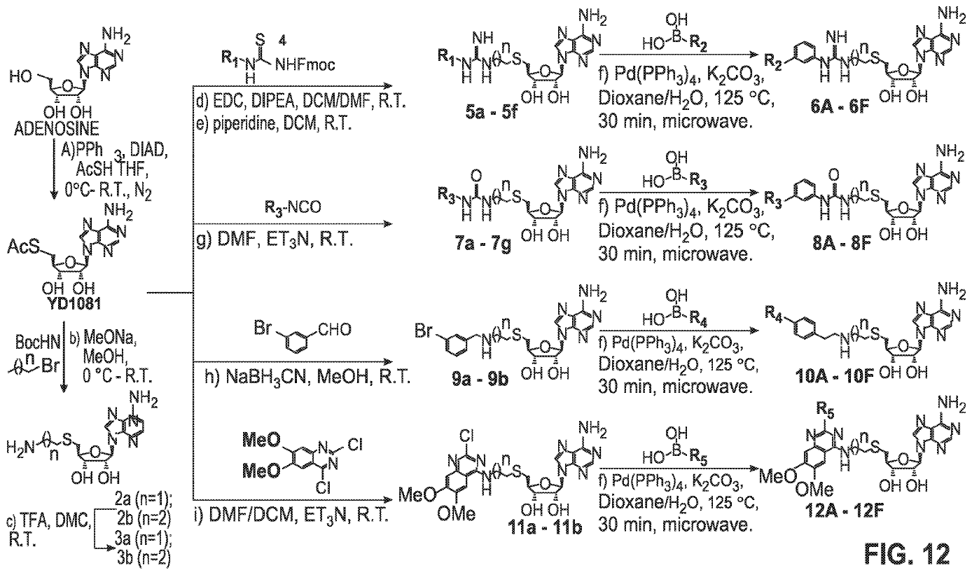

### Scheme 1. Synthesis of Analogues 6, 8, 10, and 12. (FIG. 12)

Intermediate 4 was prepared with reported methods in quantitative yields.26 Compounds 5a-5f were synthesized by condensation of the primary amine (3a or 3b) and 4, followed by deprotection. The obtained residue was washed with diethyl ether and afford 5a-5f. The primary amine (3a or 3b) readily reacted with various isocyanates in DMF. After the reaction was completed, the mixture was diluted with ethyl acetate and filtered to afford 5a-5f in high yield. Next, reductive amination of the primary amine (3a or 3b) with 3-bromophenyl aldehyde generated 9a-9b. After the reaction, the mixture was diluted with water, extracted with nbutanol/ethyl acetate (1/1), washed with brine, and concentrated to afford 9a-9b in good purity. Next, 2,4-dichloro-6,7-dimethoxyquinazoline was reacted with 3a or 3b, diluted with diethyl ether, and filtered to afford 11a-11b in high purity. Meanwhile, 5c, 5f, 7g, 9a, 9b, 11a, 11b was further derivatized with a microwave-promoted Suzuki cross-coupling reaction. Upon the completion of the reaction, the reaction mixture was acidified (pH, 3.0) with acetic acid and washed with ethyl acetate. The aqueous phase was diluted with methanol, filtered, and subjected to preparative HPLC.

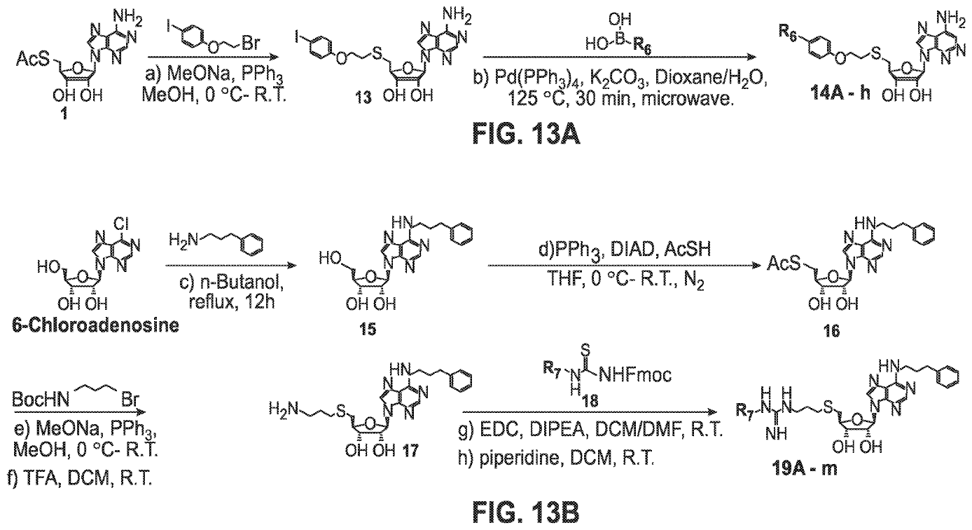

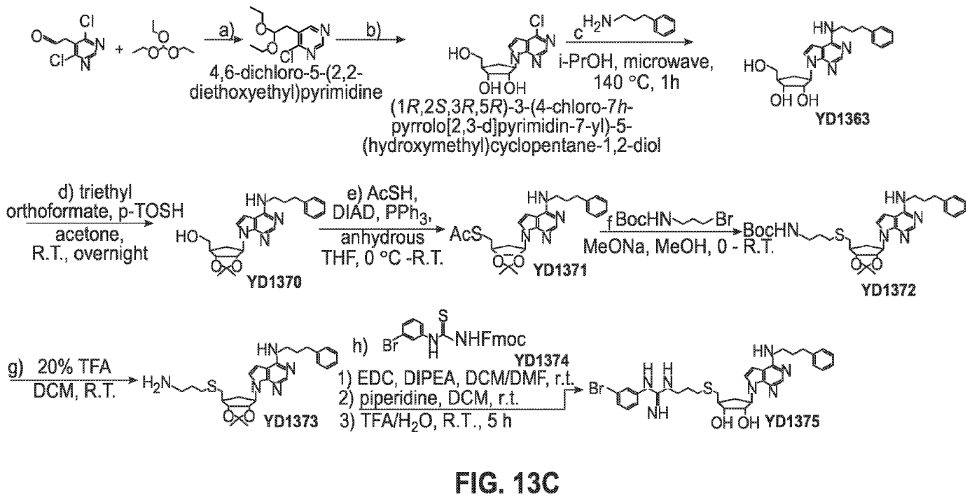

### Scheme 2. Synthesis of Analogues 14a-14f and 19a-19m (FIG. 13)

Compound 13 was prepared similarly to 3a or 3b, undergoing a microwave-promoted Suzuki cross-coupling reaction to afford 14a-14f. Finally, a similar synthetic method was applied on 6-chloroadenosine to prepare Nphenylpropyl guanidine analogues 19a-19k.

In summary, 100 adenosine analogues were facilely synthesized through a 5-step reaction sequence with no intermediate purification. Furthermore, the product was readily separated with preparative HPLC in a one-injection one compound manner to construct this focused library efficiently. Finally, all synthesized analogues in the focused library were characterized with HPLC, HRMS, and NMR to ensure quality.

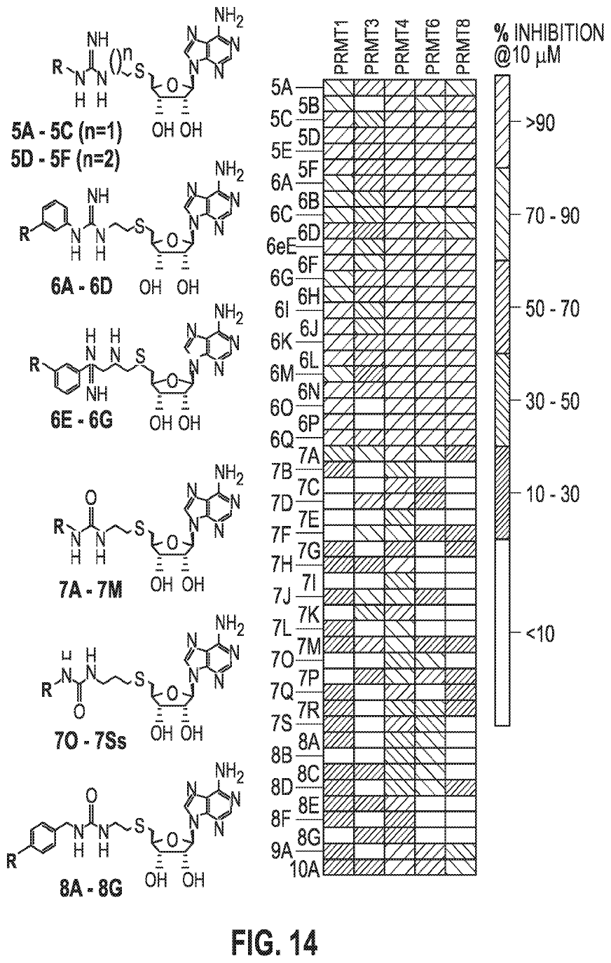

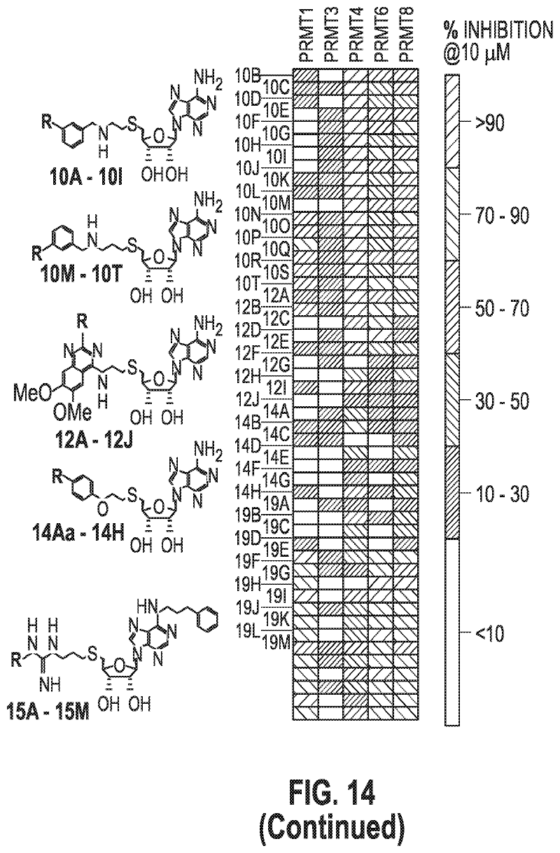

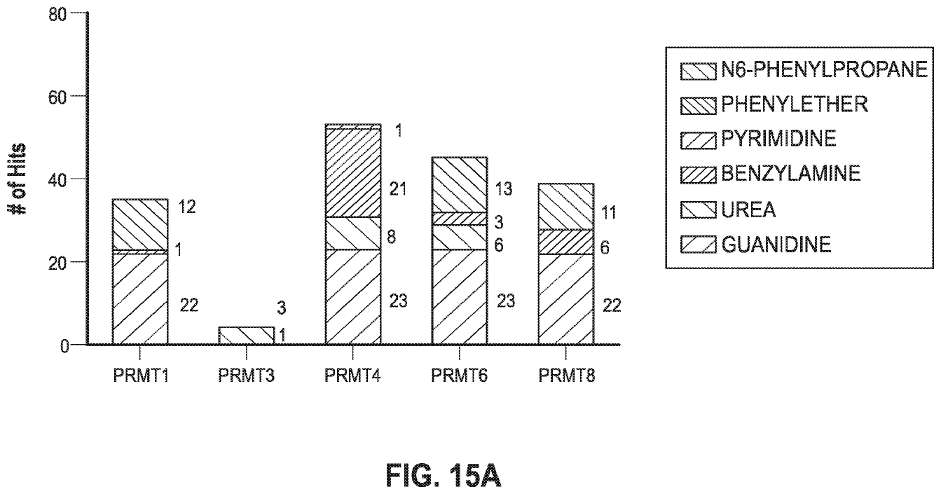

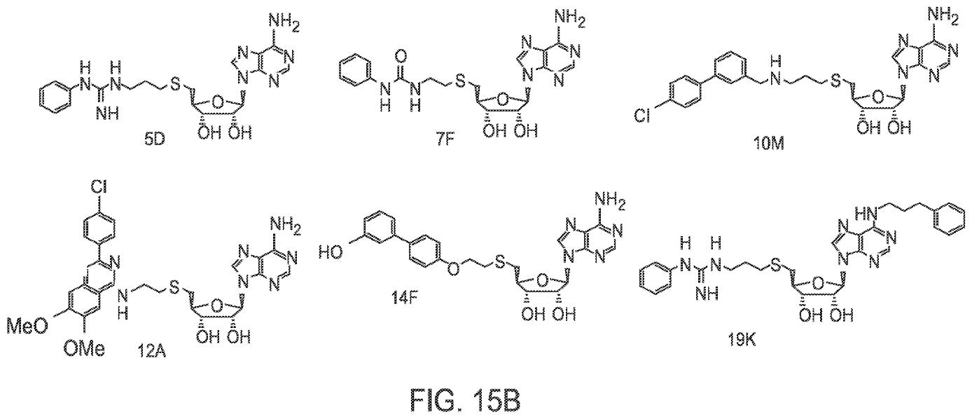

Next, the inhibition activity of the library was investigated for five PRMT members from type I PRMTs. For PRMT1 and 3, the activities at 10 μM compounds through a SAHH-coupled fluorescence assay under the Km concentration of both SAM and the respective peptide substrate were tested. For PRMT4, 6, and 8, inhibitory activities were tested with 10 μM compounds by a radioactive assay under the Km concentration of both SAM and the respective protein substrate at Reaction Biology Corp. A Heat map was generated indicating activity of the synthesized compounds (FIG. 14)

PRMT1. All guanidine analogues 5a-6q showed >50% PRMT1 inhibition at 10 μM. Among them, compounds with a propylene linker (5d-5f, 6e-6q) are slightly more potent than those with an ethylene linker (5a-5c, 6a-6d). On the contrary, all urea analogues 7a-8g showed <50% inhibition at 10 μM, suggesting the importance of imine of the guanidine group. Meanwhile, benzylamine analogues 10m-10t with a propylene linker showed higher potency than those with an ethylene result indicates replacing the guanidine with the urea group can afford selective PRMT4 inhibitor. Surprisingly, almost all benzylamine analogues 9a-10t showed strong inhibition (>90%) to PRMT4 at 10 μM and selectivity over other tested PRMTs. And no obvious inhibition differences were witnessed among analogues with different substitution groups and carbon linkers. This result indicates that benzylamine can be applied to generate a selective PRMT4 inhibitor. Only a handful of analogues in the 12 and 14 series showed moderate PRMT4 inhibition, but all these analogues demonstrated selective inhibition to PRMT4 over other tested PRMTs. Interestingly, guanidine analogues 19a-19m with an N-6 phenylpropane group showed much lower inhibition than 5a-6q, indicating strict requirement at the N-6 position of the PRMT4 SAM binding site.

PRMT6. Like PRMT4, most guanidine analogues 5a-6q showed strong inhibition (>90%) to PRMT6 at 10 μM. Although some urea compounds showed promising inhibition to PRMT6, the structures of the urea hits of PRMT6 were very different from PRMT3 and 4. Specifically, compounds 8a-8d with a nonpolar biphenyl substitution showed selective inhibition to PRMT6. While the urea hits (7d, 7f and 7j) of PRMT3 and 4 were inactive to PRMT6. Benzylamine analogues 9a-10t showed moderate to good PRMT 6 inhibition (50%-70%) at 10 μM, which suggests replacing the guanidine with the urea group will decrease PRMT6 inhibition to some degree. Only two compounds (12a and 12b) in series 12 showed selective inhibition to PRMT6 than other tested PRMTs. Analogues in series 14 did not show obvious inhibition (<50%) to PRMT6, indicating that replacing the guanidine with phenol ether is not favored. Guanidine analogues 19a-19m with an N-6 phenylpropane group showed slightly decreased inhibition (70-95%) than 5a-6q, indicating N-6 substitution can retain PRMT6 inhibition at some degree.

PRMT8. Most guanidine analogues 5a-6q showed strong inhibition (>95%) to PRMT8 at 10 μM. On the contrary, urea analogues 7a-8g did not show obvious inhibition (<30%) to PRMT8, indicating that replacing the guanidine with the urea group is not favoured. Benzylamine analogues 9a-10t showed moderate to good PRMT 8 inhibition (50%-70%) at 10 μM, which suggests replacing the guanidine with the urea group will decrease PRMT8 inhibition to some degree. Analogues in series 12 and 14 showed less than <50% to PRMT8, indicating that replacing the guanidine with either pyrimidine or phenol ether is not favored. Guanidine analogues 19a-19m with an N-6 phenylpropane group showed slightly decreased inhibition (70-95%) than 5a-6q, indicating the tractability of N-6 substitution for PRMT8 inhibition.

### Chemical Trends for PRMTs.

To focus the analysis on the trends of different sets, a “hit compound” was defined as a compound exhibiting over 70% inhibition on each PRMT (FIG. 4). The guanidine compounds showed inhibitions to all tested PRMTs, consistent with previous reports of guanidine compounds as PRMT pan inhibitors. On the other hand, phenylether compounds were the least favorable sets for type I PRMTs. PRMT3 showed higher restriction compared to other PRMTs, as PRMT3 only displayed a preference for urea compounds in addition to guanidine analogues. Meanwhile, pyrimidine compounds are selective to PRMT4. For N6 substitution, PRMT3 and 4 were not tolerant.

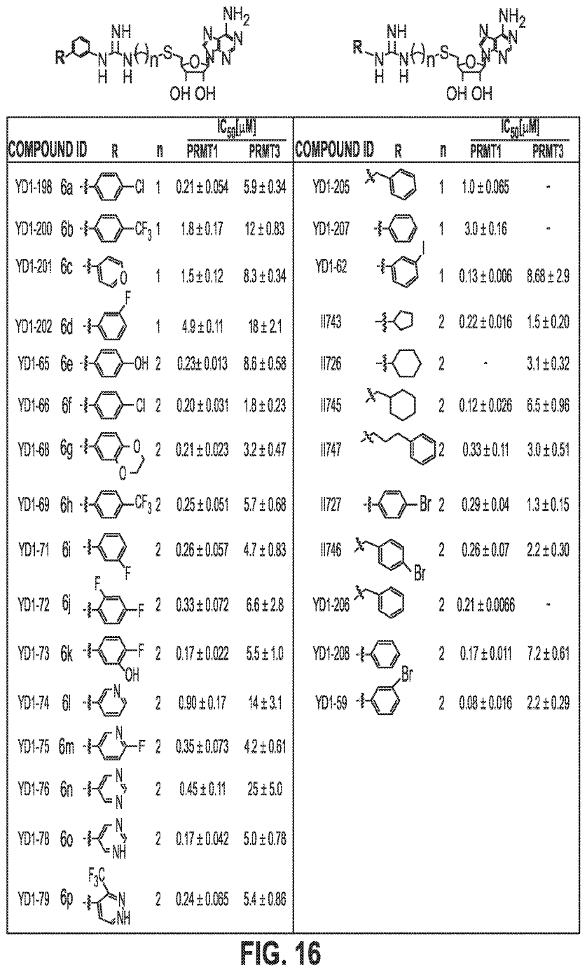

To understand the interactions of the guanidine analogues with PRMTs, it was initially attempted to obtain the co-crystal structures of PRMTs complexed with different guanidine hits but only obtained the co-crystal structures of PRMT3 complexed with the respective hits YD1208 and YD166 (FIG. 16).

The overall topology of the PRMT3-YD1208 complex clearly illustrated YD1208 as a bisubstrate analogue in the active site of PRMT3 (FIG. 16a). Structural alignment of YD1208 and SAH (PDB ID, 2FYT) suggested that the phenyl guanidine binds to the arginine binding channel rather than the homocysteine binding sites (FIG. 16b). Specifically, the thioadenosine moiety of YD1208 forms hydrogen bonds with Asp 302, Gln 303, Ile 330, and Glu 331. The phenyl guanidine group binds to the arginine binding channel by forming interactions with Glu 346 and His 496 (FIG. 16c), confirming the bisubstrate feature of inhibition. Given the high similarity of the structures of type I PRMTs, it is reasonable to predict that the guanidine analogues adopt similar binding modes in other PRMTs.

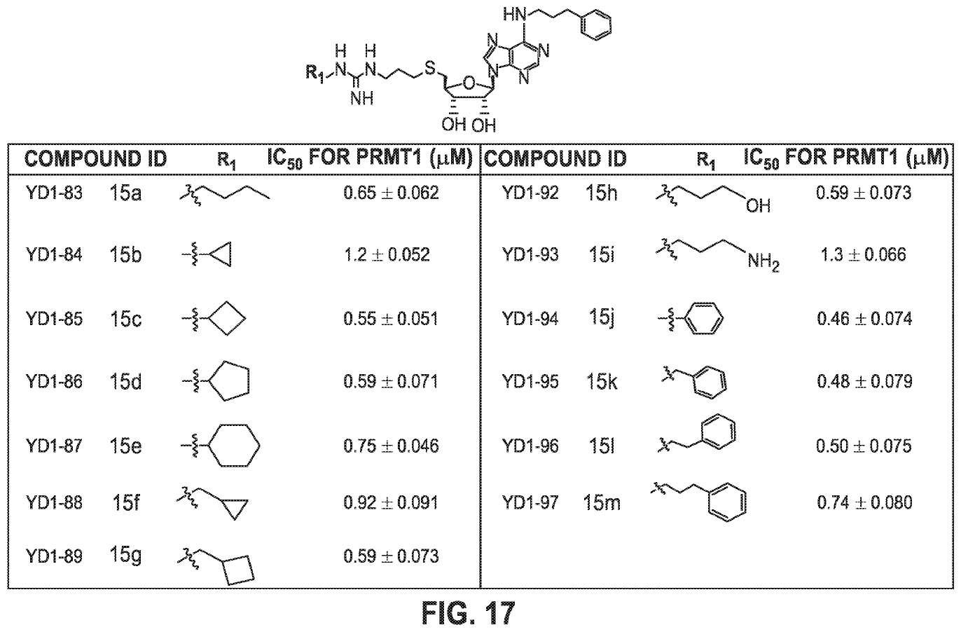

The structures of urea compounds only have one atom difference from the guanidine analogues, but showed selective inhibition to PRMT3 and 4, implying they have different binding modes from the guanidine compounds. Moreover, the urea compounds inhibit PRMT3/4 in a similar trend, suggesting urea compounds may bind at the exact location in PRMT3/4. To investigate the binding details, the co-crystal structures of PRMT3 complexed with urea compound YD1214 (7f) was determined. Surprisingly, YD1214 occupied the SAH binding site rather than the arginine binding channel (FIGS. 17a and 17b). The adenosine moiety of YD1214 remained in the same binding mode as SAH through hydrogen bonds with Asp 282, Glu 310, and Glu 311, and pi-pi interactions with Phe 218.

To investigate the binding mode of YD1214 for PRMT4, a docking study was performed by using the co-crystal structure of PRMT3-YD1214 as the template. As predicted, YD1214 resided in the SAH binding pocket of PRMT4 (FIG. 17c), similar to the pose in PRMT3. Meanwhile, urea compounds with a 2-C linker showed better inhibition to PRMT3/4 than 3-C linker ones, suggesting a limited space in the homocysteine binding sites of PRMT3/4.

Benzylamine compounds are more potent and selective inhibition to PRMT4 than other tested PRMTs. Compound 10m (SGC8172) was previously reported as a potent PRMT4/7 inhibitor, consistent with previous screening results. And selective PRMT7 inhibition was achieved by increasing the carbon linker of 10m from 3-C to 4-C(SGC8158), implying the linker length can affect the PRMT 4 inhibition. Compound 10a, a close analogue of 10m but with a 2-C linker, showed equal potency on PRMT4. Compounds 10a and 10m showed more potent PRMT4 inhibition activity than SGC8158, suggesting that 2-C and 3-C linkers can fit the binding site of PRMT4 better than 4-C linker. To understand the molecular interaction, 10m on PRMT4 was docked by using the reported co-crystal structure of PRMT7-SGC8158 as a template. In FIG. 17d, the predicted binding pose of 10m displayed similar interactions as SAH for the adenosine moiety, suggesting the reliability of the docking result. Meanwhile, the benzylamine moiety appeared to bind at the peptide binding sites rather than the homocysteine site, similar to the binding pose of SGC8158 in PRMT7.

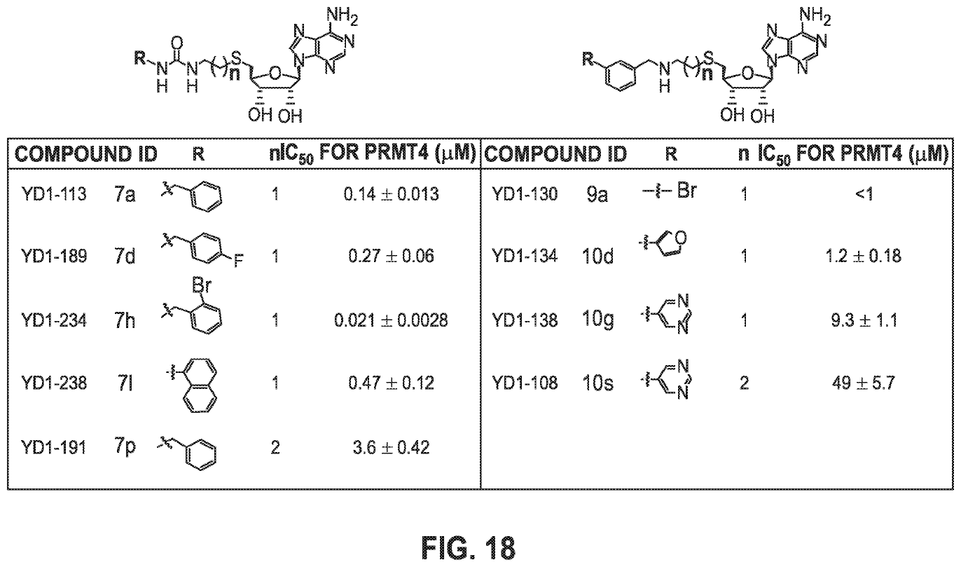

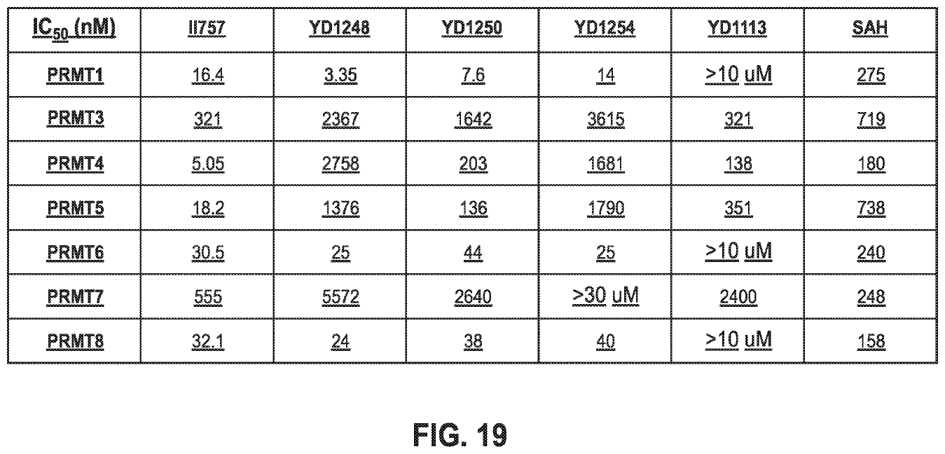

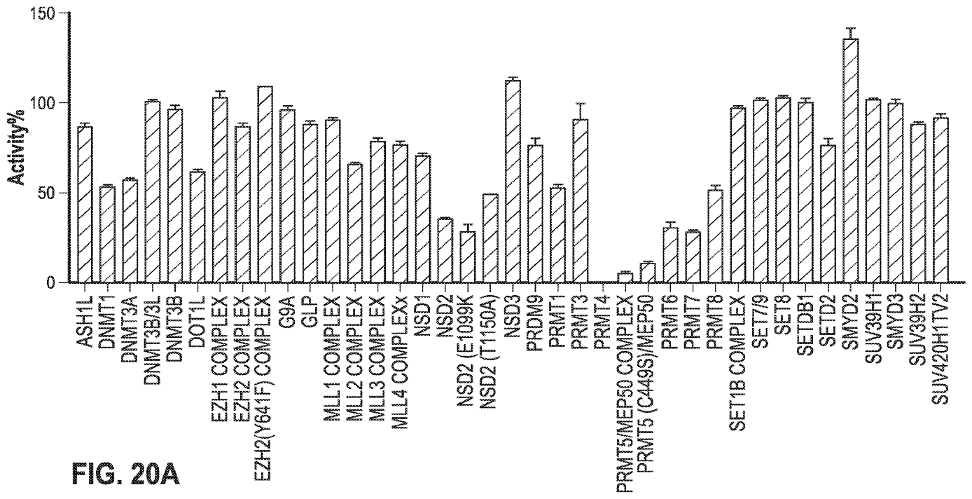

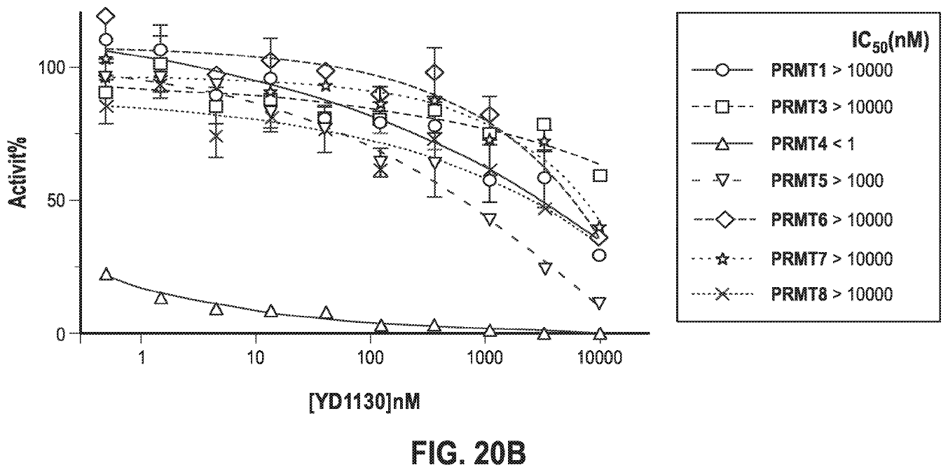

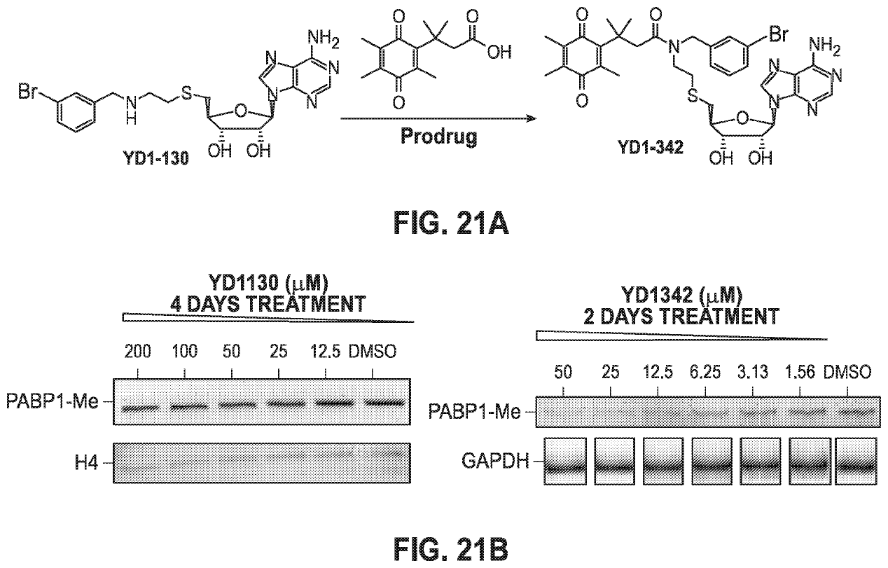

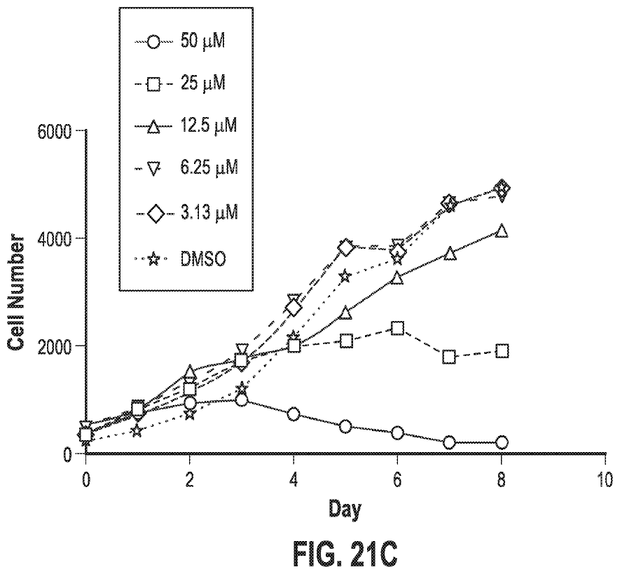

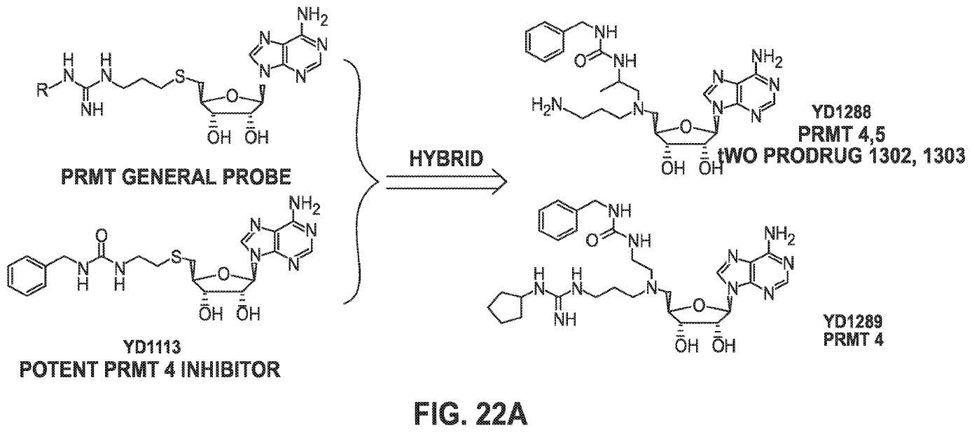

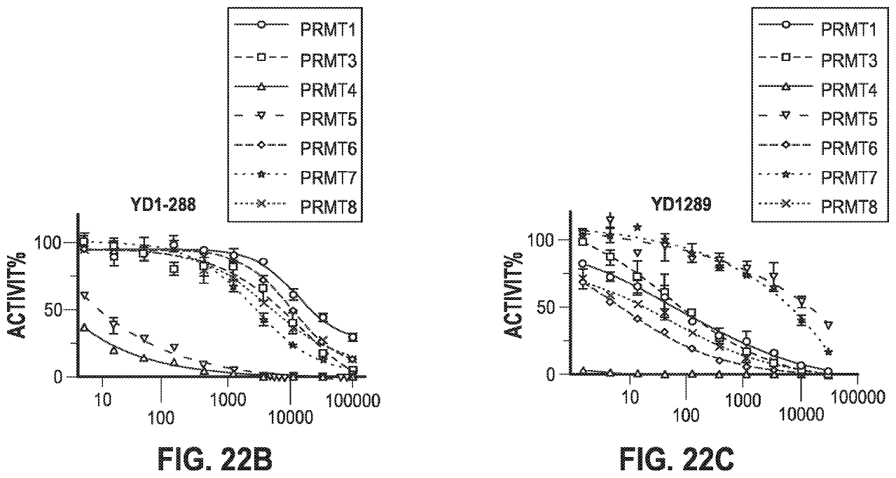

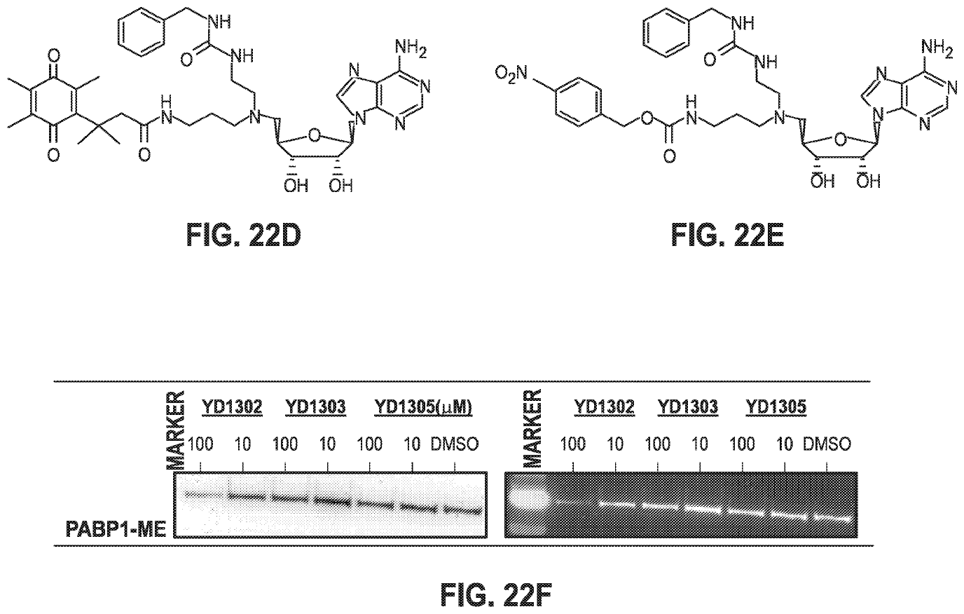

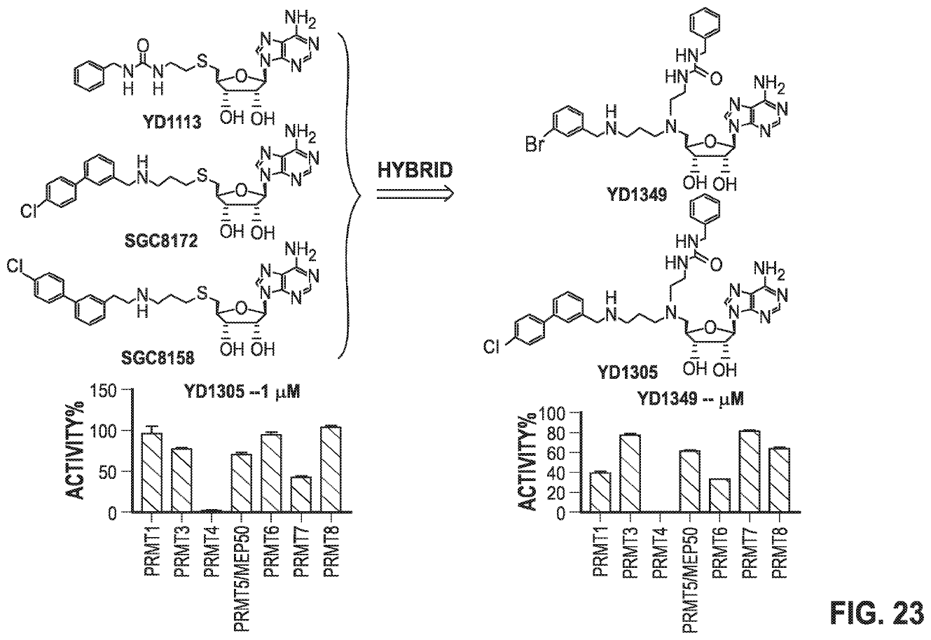

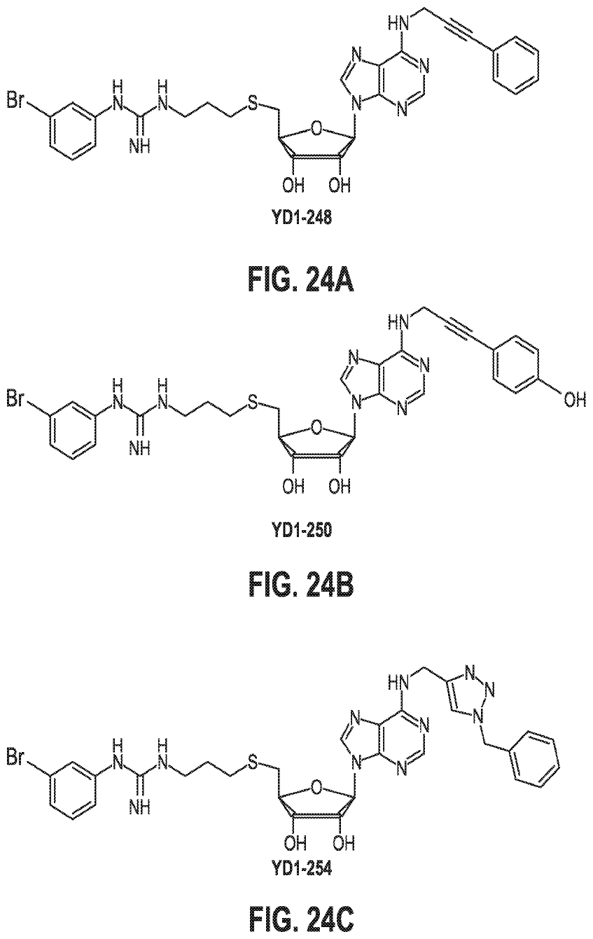

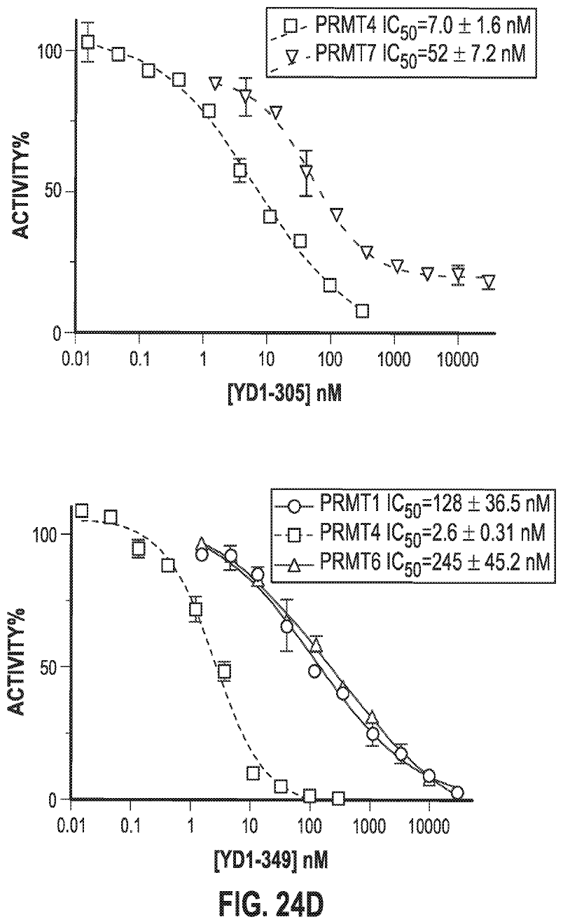

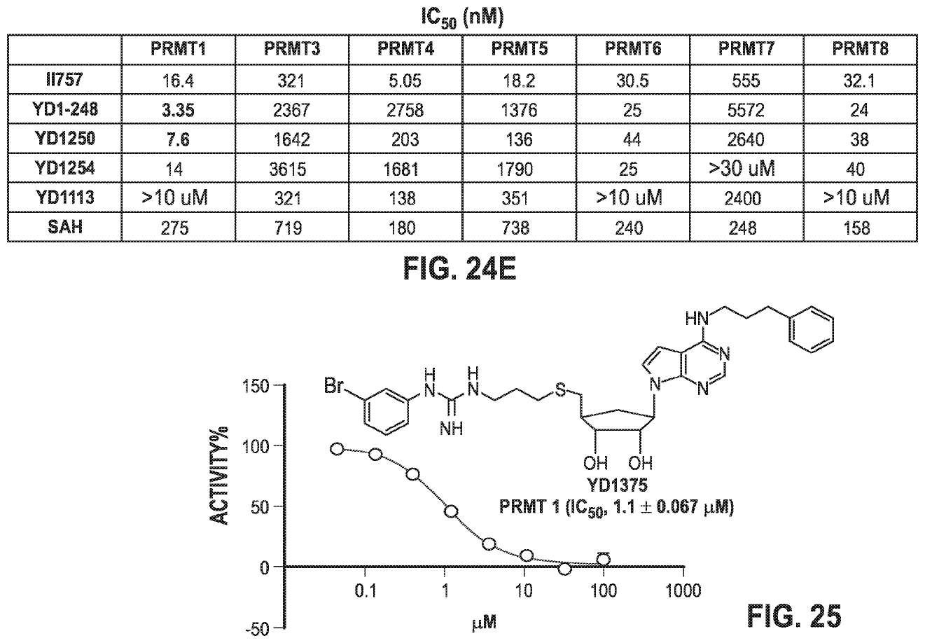

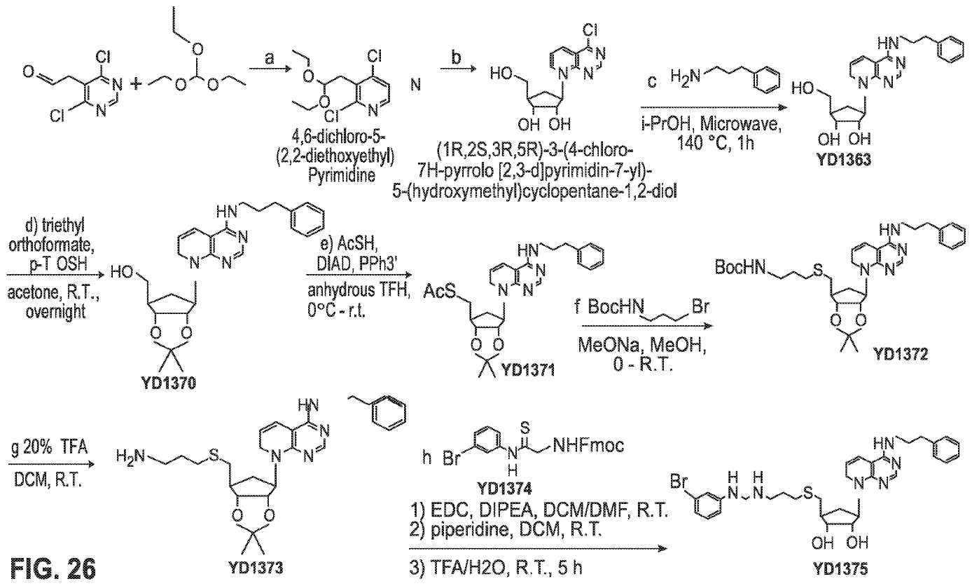

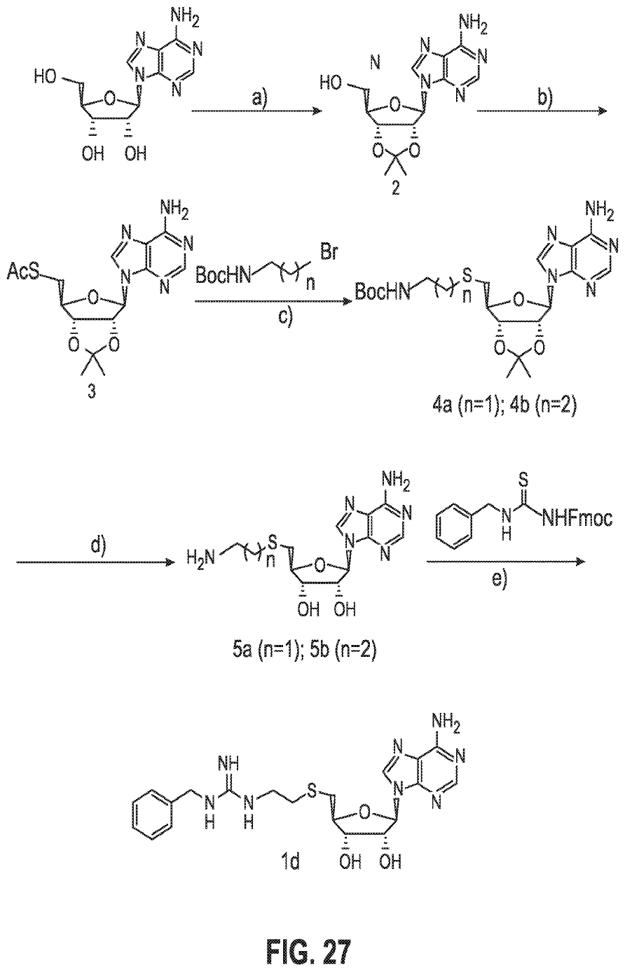

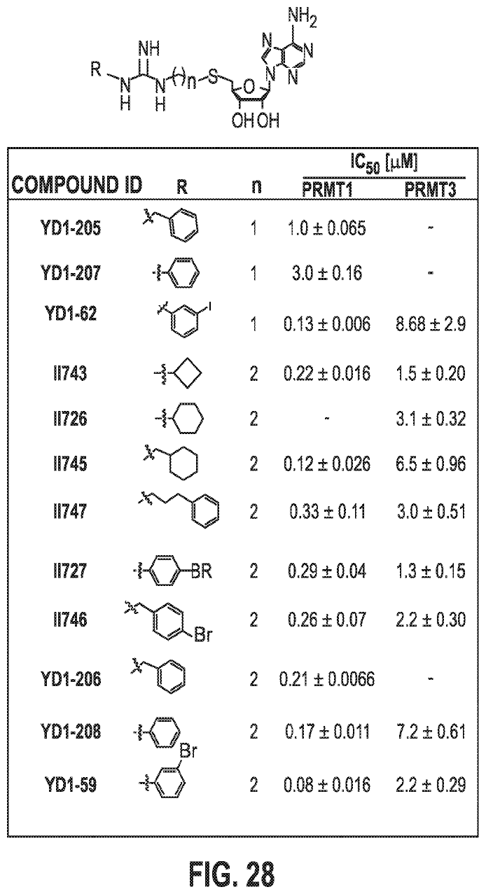

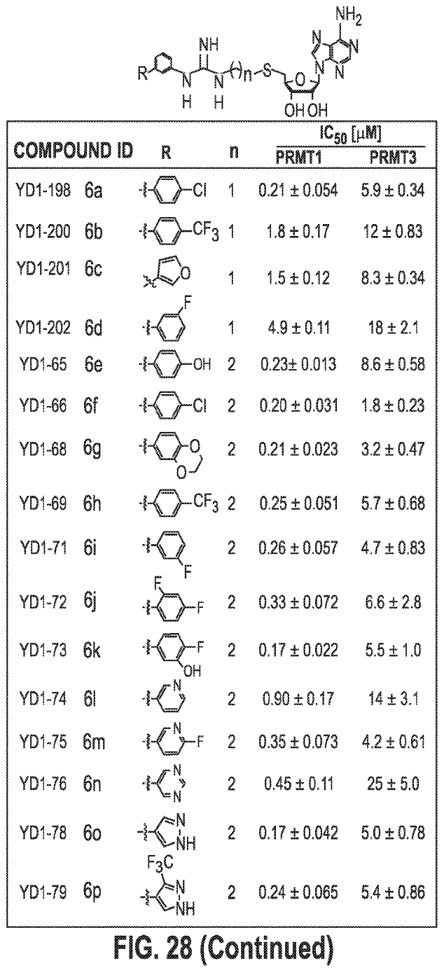

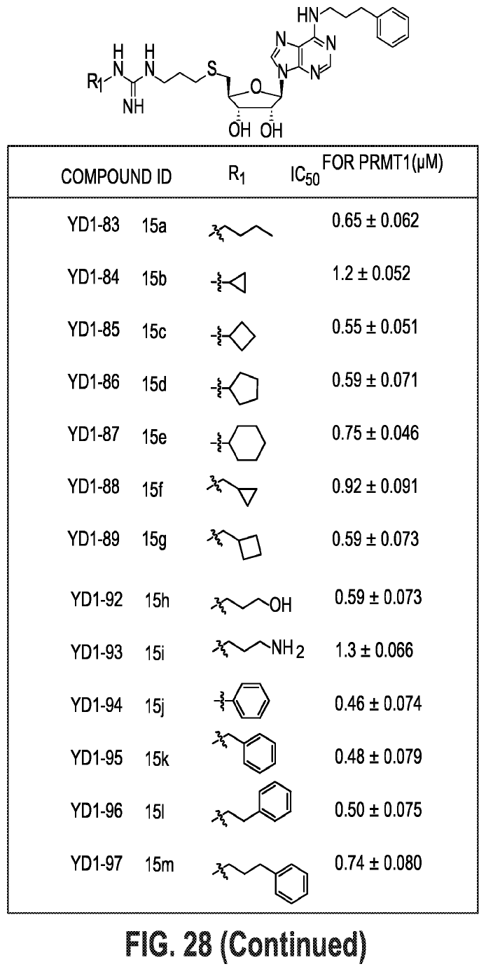

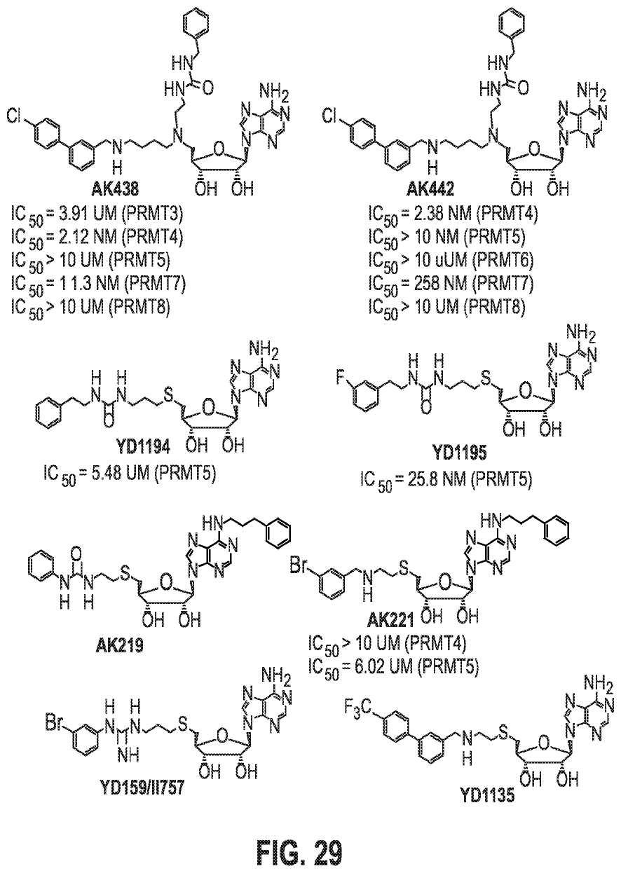

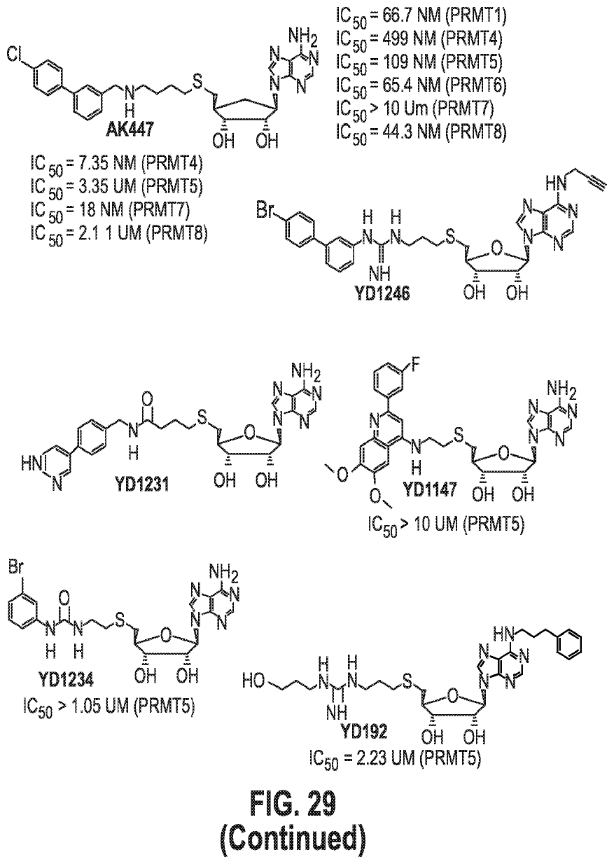

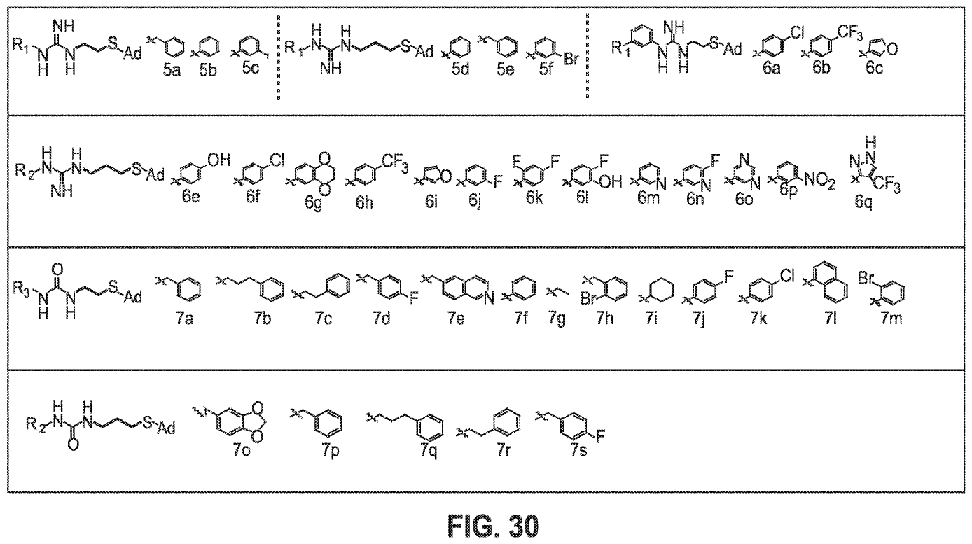

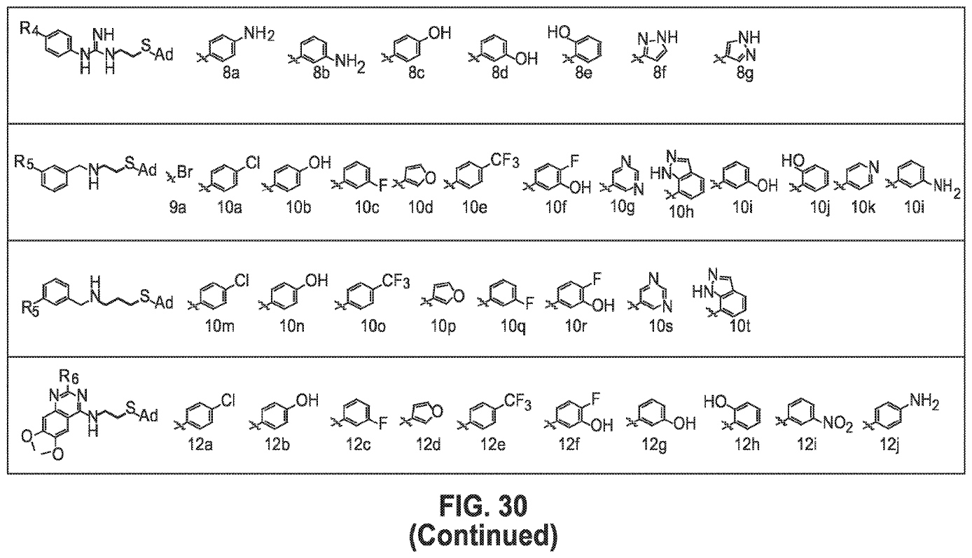

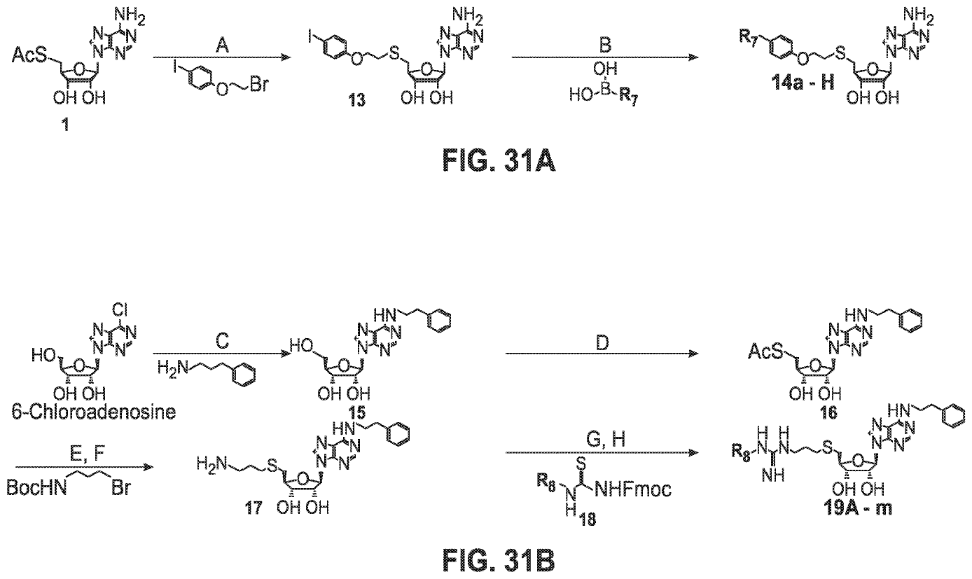

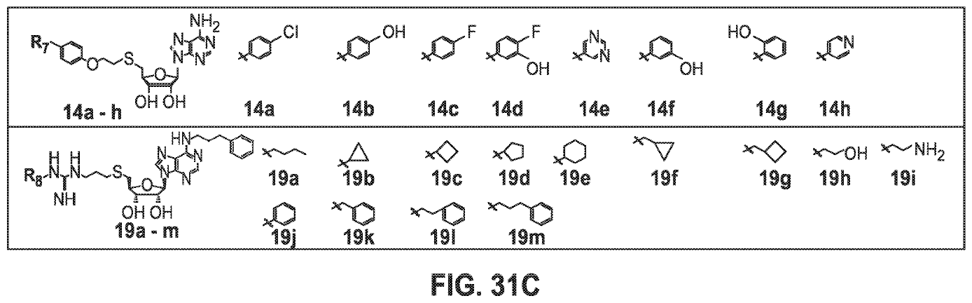

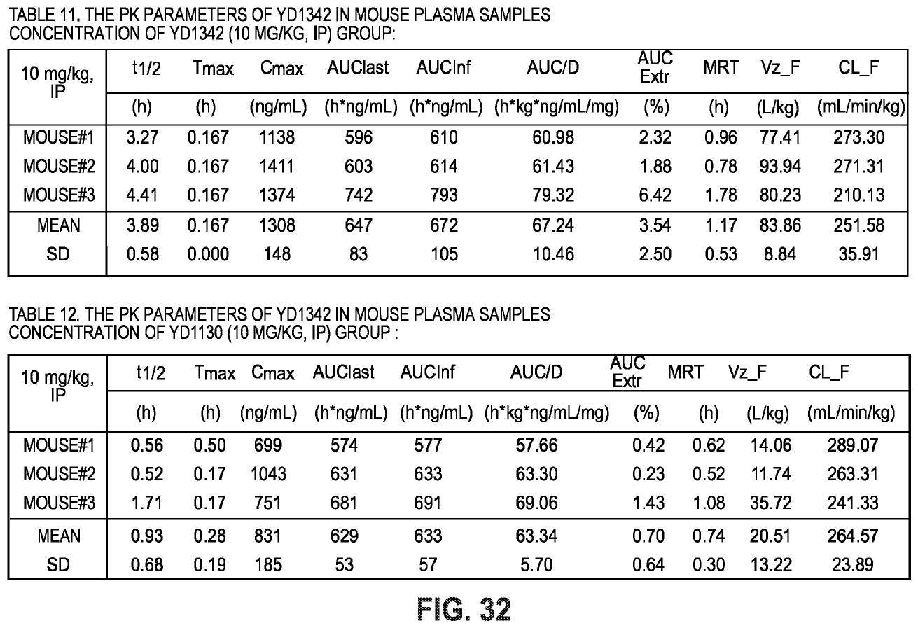

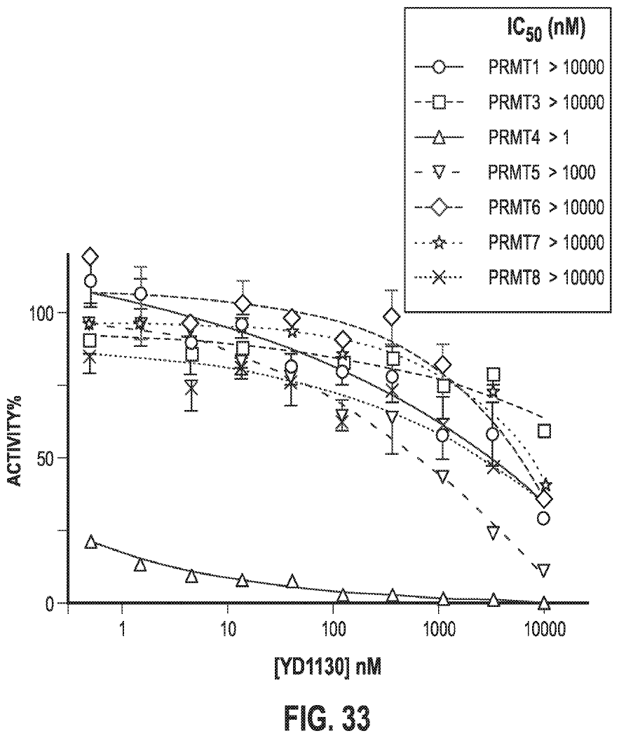

The guanidine compounds showed general inhibition to all tested PRMTs. However, introducing a phenylpropyl group at the N-6 position exhibited different inhibition. Notably, the inhibition potency to PRMT3 and 4 was decreased but remained for PRMT1/6/8. One possible reason may be due to the buried SAH binding sites of PRMT3 and 4 (FIGS. 16 and 17). On the contrary, an open-spaced channel can be found around the N-6 position of SAH binding sites in PRMT1/6/8. As shown in FIG. 18, YD194 adopted similar binding poses with guanidine analogues in PRMT1/6/8 from docking studies, except that the phenyl propyl group binds at a channel near the N-6 position of adenosine as expected.

### General Experimental Procedure

The commercial reagents were used as received. All 1H and 13C NMR spectra were recorded on a Brucker 500 MHz spectrometer. HRMS spectra were recorded on an Agilent high-resolution 6550 quadrupole time-of-flight (Q-TOF) LCMS instrument. Samples were analyzed on Agilent 1260 HPLC systems. The mobile phase consisted of buffer A (ultrapure H2O containing 0.1% TFA and 0.1% CH3CN) and buffer B (chromatographic grade CH3CN containing 0.1% HCOOH) was applied at a flow rate of 1 mL min-1. All compounds used for biochemical assay possessed a purity of at least 95% based on HPLC analysis.

### Chemistry

### Synthesis of Compound 1

To a stirring solution of PPh3 (5.24 g, 20.0 mmol) in anhydrous THE (250.0 mL) under nitrogen was added DIAD (4.04 g, 20.0 mmol) in the ice bath and stirred for 1 hour. Then, adenosine (2.67 g, 10.0 mmol) was added and stirred for another 2 hours. Next, thiol acetic acid (1.14 g, 15.0 mmol) was added. The reaction was slowly raised to room temperature and stirred for 24 h. After the reaction, the mixture was filtered and concentrated under reduced pressure. The crude product was dissolved in 500 mL water (pH, 2.0) and washed with 3×300 mL EA/hexane (1:5). Next, the aqueous layer was basified with saturated aqueous NaHCO₃, and extracted with 3×300 mL EA. Combined organic layers were washed with brine, dried over anhydrous sodium sulfate, and concentrated under reduced pressure to afford 1 (1.69 g, 52%) in high purity, which was directly used in the next step.

### General Procedure for Synthesis of 2a and 2b

To a solution of compound 1 (975.0 mg, 3.0 mmol) and tert-butyl (2-bromoethyl)carbamate or tert-butyl (3-bromopropyl)carbamate (4.5 mmol) in anhydrous methanol (50.0 mL) was added sodium methoxide (30% in methanol, 1.62 mL, 9.0 mmol) at 0° C. After the reaction mixture was stirred overnight, 1.0 mL acetic acid and 300 mL water was added. The mixture was washed with 3×100 mL EA/hexane (1:10). Next, aqueous layer was basified with saturated aqueous NaHCO₃, and extracted with 3×150 mL EA. Combined organic layers were washed with brine, dried over anhydrous sodium sulfate, and concentrated under reduced pressure to afford 2a (805 mg, 63%) or 3a (911 mg, 69%) in high purity, which was directly used in the next step.

### Synthesis of 3a and 3b

[To a stirred solution of compound 2a (852 mg, 2.0 mmol) or 2b (880 mg, 2.0 mmol) in 10 ml DCM was slowly added 10 ml TFA. The reaction mixture was stirred at room temperature for 4 h and concentrated under reduced pressure. The generated crude oil was washed with 3×30 ml Et2O and concentrated to afford 3a (593 mg, 91%) or 3b (605 mg, 89%) as an oil.

### General Procedure for Synthesis of Thiourea 4

To a stirring solution of Fmoc-thioisocynate (843 mg, 3.0 mmol) in 30 ml DCM was slowly added a solution of amine (654 mg, 3.0 mmol) in 20 ml of DCM in the ice bath. The mixture was stirred for 1-4 h and concentrated under reduced pressure to generate an oil, which was directly used in the next step.

### General Procedure for Synthesis of 5

To a stirring solution of 3a (163 mg, 0.5 mmol) or 3b (170 mg, 0.5 mmol) in anhydrous DCM/DMF (8/2 ml) was added thiourea 4 (0.6 mmol), DIPEA (97 mg, 0.75 mmol) and EDC (78 mg, 0.5 mmol) at room temperature. The reaction mixture was stirred overnight and diluted with EA. After washed with brine, the organic layer was concentrated under reduced pressure. Then the residue was dissolved in 10 ml DCM and 10 ml piperidine, stirred for 2 h at room temperature, and concentrated under reduced pressure. The residue was washed with 3×30 mL EA and dissolved in 4 mL of methanol, which was separated with prep-HPLC (MeCN/H2O) to afford 5a-5f (38%-70%).

### General Procedure for Synthesis of 6

To a stirring solution of 5c or 5f (0.07 mmol), phenylboronic acid (0.09 mmol), dioxane (1.6 mL) and H2O (0.4 mL) were added Pd(PPh3)4 (8.0 mg, 0.007 mmol) and K2CO3 (29 mg, 0.21 mmol). The resulting solution was stirred inside a microwave at 125° C. for 30 min. After cooling, the volatiles were removed under vacuo. The residue was redissolved in methanol and purified by HPLC (MeCN/H2O) to give 6a-6q (45%-75%).

### General Procedure for Synthesis of Urea Compounds 7

To a stirring solution of 3a (163 mg, 0.5 mmol) or 3b (170 mg, 0.5 mmol) in 5 mL anhydrous DMF was added TEA (152 mg, 1.5 mmol) and isocyanate (0.6 mmol) at room temperature. After stirred at room temperature for 1-4 h, the mixture was diluted with 100 mL Et2O and centrifuged. Then the residue was dissolved in 5 mL methanol for purdification with prep-HPLC (MeCN/H2O) to afford 7a-7s (56%-88%).

### General Procedure for Synthesis of Compounds 8

Compounds 8a-8f (45%-78% yield) were synthesized by following the same methods as 6.

### General Procedure for Synthesis of 9

To a stirring solution of 3a (163 mg, 0.5 mmol) or 3b (170 mg, 0.5 mmol) in 5 mL methanol was added 3-bromobenzenealdehyde (96 mg, 0.53 mmol) and sodium cyanoborohydride (40 mg, 0.6 mmol). The reaction mixture was stirred at room temperature overnight. The mixture was filtered and separated with prep-HPLC (MeCN/H2O) to afford benzylamine compounds 9a and 9b (81%-86%).

### General Procedure for Synthesis of 10

Compounds 10a-10t (43%-82%) were synthesized by following the same method as 6.

### Synthesis of 11

To a stirring solution of 3a (170 mg, 0.5 mmol) in anhydrous DCM/DMF (8/2 ml) was added 2,4-Dichloro-6,7-dimethoxyquinazoline (142 mg, 0.55 mmol) and TEA (152 mg, 1.5 mmol) at room temperature. The reaction mixture was stirred overnight. After removal of the volatiles under vacuo, the mixture was diluted with 30 mL Et2O and centrifuged to afford 11 (227 mg, 83%).

### General Procedure for Synthesis of 12

Compounds 12a-12j (31%-64%) were synthesized similarly as 6.

### Synthesis of 13

To a stirring solution of 3a (905 mg, 2.78 mmol) in 30 mL anhydrous methanol was added 1-(2-bromoethoxy)-4-iodobenzene (978.0 mg, 3.0 mmol) and sodium methoxide (30% in methanol, 5.4 mL, 8.34 mmol) at 0° C. The reaction mixture was stirred overnight with the temperature slowly increasing to room temperature. At the end of the reaction, 4 mL acetic acid was added, and the volatiles were removed under vacuo. The residues were dissolved in 200 ml water, washed with hexane (3×100 mL), and extracted with EA (3×150 mL). The combined organic layers were washed with water and brine, dried over anhydrous sodium sulfate, and concentrated under reduced pressure to afford 13 (1147 mg, 78%).

### General Procedure for Synthesis of Compounds 14

Compounds 14a-14f (54%-81% yield) were synthesized similarly as 6.

### Synthesis of 15

A mixture of 3-phenylpropylamine (395 mg, 1.4 mmol), 6-chloropurine riboside (143 mg, 0.5 mmol), and TEA (2 mL, 12.0 mmol) in 1-propanol (25 mL) was heated at 70° C. overnight. The mixture was concentrated under reduced pressure and triturated with H2O to give white precipitates, which were filtered to yield 15 (151 mg, 81%).

### Synthesis of 16

To an ice-cold solution of triphenylphosphine (2.62 g, 10.0 mmol) in dry THE (100 mL), diethyl azodicarboxylate (1.6 mL, 10 mmol) was added dropwise over 5 min and stirred for 30 min. Then 15 (1.92 g, 5.0 mmol) was added and continued to stir for 2 h. To the resulting yellow suspension was slowly added a solution of thioacetic acid (0.7 mL, 10.0 mmol) in dry THE (5 mL) and stirred overnight while the temperature slowly increased to room temperature. Then the mixture was filtered and the filtrates were concentrated under reduced pressure. The crude product was dissolved in 500 mL water (pH, 2.0) and washed with 3×300 mL EA/hexane (1:9). Next, the aqueous layer was basified with saturated aqueous NaHCO₃ and extracted with 3×300 mL EA. The combined organic layers were washed with brine, dried over anhydrous sodium sulfate, and concentrated to afford 16 (1.57 g, 71%) for direct usage.

### Synthesis of 17

To a stirring solution of 16 (445 mg, 1.0 mmol) and tert-butyl (3-bromopropyl)carbamate (1.5 mmol) in dry methanol (20 mL) was added sodium methoxide (30% in methanol, 0.6 mL, 3.0 mmol) under an argon atmosphere. A mixture of 1.0 mL acetic acid and 300 mL water was added to quench the reaction. The reaction mixture was washed with 3×100 mL EA/hexane (1:10). Next, the aqueous layer was basified with saturated aqueous NaHCO₃, and extracted with 3×150 mL EA. combined organic layers were washed with brine, dried over anhydrous sodium sulfate, and concentrated to afford an oil, which was dissolved in DCM (5 mL) and TFA (5 mL) and stirred for 4 h at room temperature. The volatiles were removed under reduced pressure to afford the desired product 17 (352 mg, 77%).

### Synthesis of 18

To a stirring solution of Fmoc-isothiocyanate (140 mg, 0.5 mmol) in 25 mL DCM was slowly added the respective amines (0.5 mmol) at 0° C. The reaction mixture was stirred at room temperature for 1 h. The volatiles were removed under reduced pressure to afford 18 in quantitative yield, which was directly used for the next step.

### General Procedure for Synthesis of Compounds 19

Compounds 19a-19m (38%-64% yield) were synthesized similarly as 6.

### Synthesis of YD1375

(1) To a stirred solution of (1R,2S,3R,5R)-3-(4-chloro-7H-pyrrolo[2,3-d]pyrimidin-7-yl)-5-(hydroxymethyl)cyclopentane-1,2-diol (283 mg, 1.0 mmol) in 2-propanol 3 mL was added with 3-phenylpropan-1-amine (0.5 mL) and irradiated with microwave at 140° C. for 1 hour under nitrogen. When the reaction was complete, the mixture was separated with flash column (DCM/MeOH) to afford YD1363 (184 mg, 65%).

(2) p-Toluenesulfonic acid monohydrate (34 mg, 0.2 mol) and triethyl orthoformate (30 mg, 2.0 mmol) were subsequently added into the solution of adenosine YD1363 (153 mg, 0.4 mmol) in 10 mL acetone. The resulted mixture was stirred at room temperature overnight. After neutralization with saturated solution of sodium carbonate, the solution was extracted with EA (3×50 mL). The extracts were combined, washed with brine, dried with anhydrous sodium sulfate. The volatiles were removed under low pressure. The generated oil was separated with combiflash (DCM/MeOH, 10/1) to afford YD1370 (132 mg, 78%).

(3) To a stirred solution of PPh3 (262 mg, 1.0 mmol, 2.0 eq) in anhydrous THE (10 mL) under nitrogen was added DIAD (202 mg, 1.0 mmol, 2.0 eq) at ice bath and stirred for 1 hour. Then, YD1370 (211 mg, 0.5 mmol, 1.0 eq) was added and stirred for another 2 hours. Next, thiol acetic acid (56 mg, 0.75 mmol, 1.5 eq) was added. The reaction was slowly raised to room temperature and stirred for 24 hours. After the reaction was finished, the mixture was filtered, and the filtrates was evaporated under vacuum. The generated oil was separated with combiflash (DCM/MeOH, 10/1) to afford YD1371 (180 mg, 75%).

(4) To a stirred solution of YD1371 (240 mg, 0.5 mmol) in anhydrous MeOH (10 mL) under nitrogen was added tert-butyl (3-bromopropyl)carbamate (143 mg, 0.6 mmol) and sodium methoxide (30% in methanol, 539 μl, 3.0 mmol) at 0° C. The reaction mixture was stirred overnight. At the end of the reaction, acetic acid (0.5 ml) was added, and the solvent was removed under vacuo. The residues were dissolved in 50 mL water and extracted with EA (3×50 mL). The extracts were combined, washed with water and dried with anhydrous sodium sulfate. The volatiles were removed under low pressure. The generated oil was separated with combiflash (DCM/MeOH, 10/1) to afford YD1372 (241 mg, 81%).

(5) To a stirred solution of YD1372 (238 mg, 0.4 mmol) in DCM (10 mL) was added 2 mL TFA. The reaction mixture was stirred for 4 h. At the end of the reaction, the volatiles were removed under vacuo. The residues were washed with cold Et2O (3×50 mL). The residues were dissolved in methanol and adjusted pH with 2M NaOH. After evaporation of the solvent, the crude product was washed with Hexane and directly used in the next step.

(6) To a solution of YD1373 (198 mg, 0.4 mmol) and YD1374 (272 mg, 0.6 mmol) in anhydrous DCM/DMF (8/2 mL) was added DIPEA (155 mg, 1.2 mmol) and EDC (154 mg, 0.8 mmol) at room temperature. The reaction mixture was stirred overnight. At the end of the reaction, the mixture was diluted with EA and washed with brine. The solvent was removed under vacuo. The residue was dissolved in 10 mL DCM and 10 mL piperidine. The mixture was stirred for 2 h at room temperature. The solvent was removed under vacuo. The residues were washed with diethyl ether (2×30 mL) and dissolved in 10 mL of TFA containing 10% water, which was stirred at room temperature for 5 h. After that, the solvent was removed under nitrogen and dissolved in 2 mL MeOH, which was separated with preparative HPLC (MeCN/H2O) to afford YD1375 (107 mg, 41%).

### Biochemical Assay

### Protein Expression and Purification

PRMT1, PRMT3, PRMT4 and SAHH were expressed and purified as reported before.

A fluorescence-based SAHH-coupled assay was applied to determine the inhibition activity of all synthesized compounds on PRMT 1 and 3. The PRMT1 methylation assay was performed under the following conditions in a final well volume of 100 μL: 2.5 mM HEPES (pH=7.0), 25 mM NaCl, 25 μM EDTA, 50 μM TCEP, 0.01% Triton X-100, 5 μM SAHH, 0.1 μM PRMT1, 10 μM AdoMet, and 10 μM ThioGlo4. After incubating with the inhibitors for 10 min at 37° C., reactions were initiated by the addition of 5 μM H4-21 peptide (Km value). The fluorescence signal was monitored on a BMG CLARIOstar microplate reader with excitation at 400 nm and emission at 465 nm for 15 min. Data were processed using GraphPad Prism software 8.0. All experiments were performed in duplicate.

The PRMT3 methylation assay was performed under the following conditions in a final well volume of 100 μL: 20 mM Tris (pH=7.5), 0.01% Triton X-100, 5 μM SAHH, 0.25 μM PRMT3, 30 μM AdoMet, and 15 μM ThioGlo4. After incubating with the inhibitors for 10 min at 30° C., reactions were initiated by the addition of 30 μM H4-21 peptide (Km value). Fluorescence was monitored on a BMG CLARIOstar microplate reader with excitation 380 nm and emission 505 nm. Data were processed by using GraphPad Prism software 8.0.

The PRMT 4, 6 and 8 methylation assays were performed by Reaction Biology corp.

Other compounds that can be synthesized using the methods described herein include:

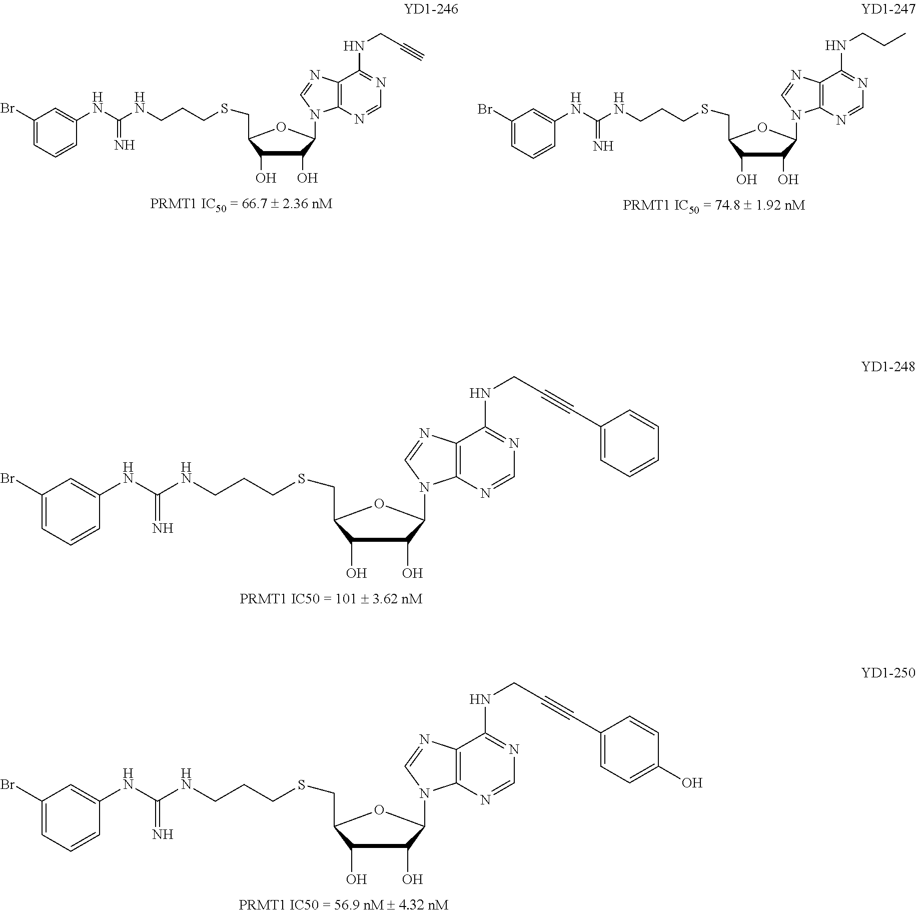

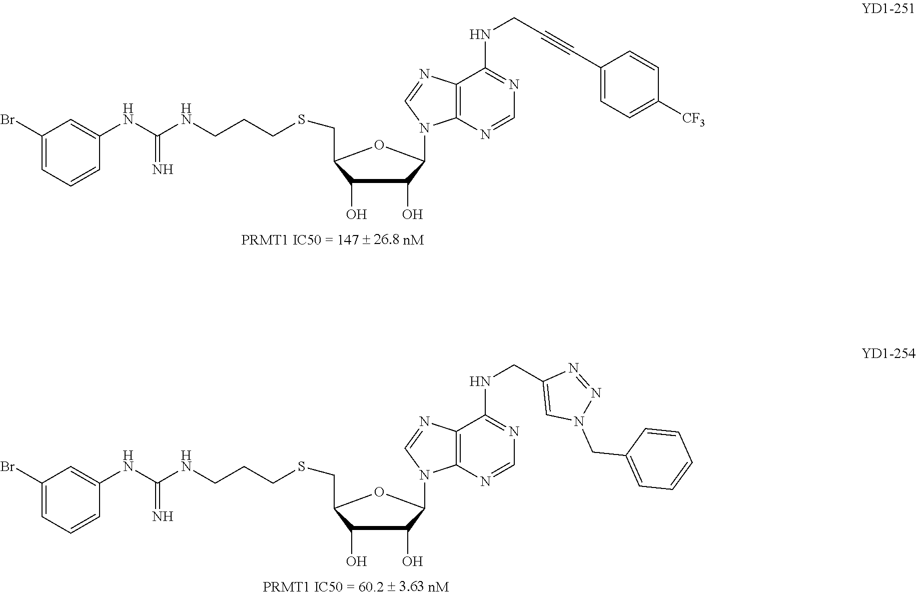

and the compounds in FIG. 25 (synthesis of YD1375).

### Synthesis of YD1375

Compound A was synthesized as previously reported. A mixture of 2-(4,6-dichloropyrimidin-5-yl)acetaldehyde (5 g, 26.1 mmol), triethoxymethane (4.65 g, 1.2 mmol), and Ts-OH (298.7 mg, 0.06 mmol) in EtOH (30 mL) was stirred at 40° C. for 2 h. After completion of the reaction, aqueous Na2CO3 was added to the mixture to adjust pH to 8. The solvent was removed under reduced pressure and the residue extracted with EtOAc (3×), washed with water (10 ml). The organic layer was dried over anhydrous Na2SO4, filtered, and concentrated under reduced pressure to give the residue that was purified by column chromatography on silica gel to afford compound 1 (5.2 g, 75%) as a clear oil.

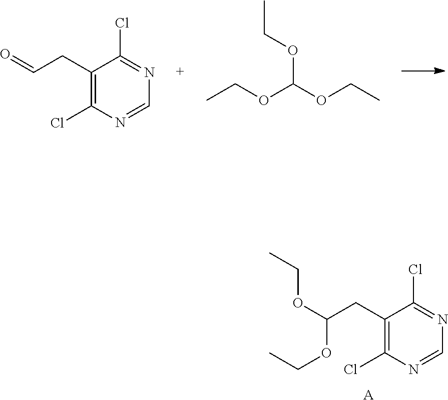

Compound B was synthesized following an earlier reported procedure. To a mixture of 1 (600 mg, 2.30 mmol) and (1S,2R,3S,5S)-3-amino-5-(hydroxymethyl cyclopentane-1,2-diol. HCl (464 mg, 2.53 mmol) in IPA: H2O (7:1) 10 mL was added TEA (800 μL, 5.74 mmol) in one portion at rt under N2. The reaction was heated to 90° C. and stirred for 23 h. The mixture was cooled to 50° C. and 4 M HCl (1.15 mL, 4.59 mmol) was added slowly. The reaction was then stirred at 50° C. for 2 h. The reaction was cooled to rt and NaHCO₃ was added slowly. The reaction mixture was extracted with EA, dried with Na2SO4, filtered, and purified by flash column eluting with 8% MeOH in DCM to afford compound 2 (430 mg, 67%) as a yellow solid.

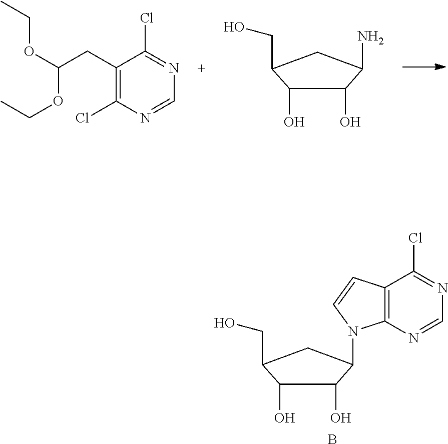

To a stirred solution of (1R,2S,3R,5R)-3-(4-chloro-7H-pyrrolo[2,3-d]pyrimidin-7-yl)-5-(hydroxymethyl)cyclopentane-1,2-diol (283 mg, 1.0 mmol) in 2-propanol 3 mL was added with 3-phenylpropan-1-amine (0.5 mL) and irradated with microwave at 140° C. for 1 hour under nitrogen. When the reaction was complete, the mixture was separated with flash column (DCM/MeOH) to afford YD1-363 (184 mg, 65%).

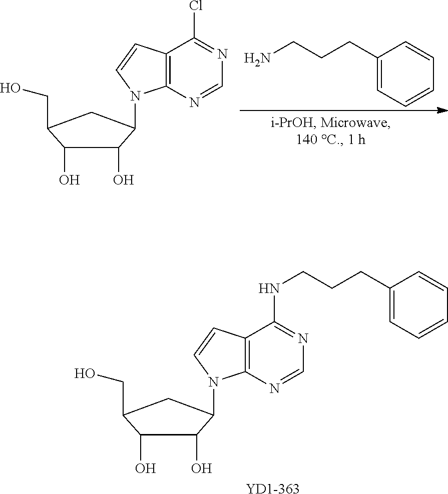

p-Toluenesulfonic acid monohydrate (34 mg, 0.2 mol) and triethyl orthoformate (30 mg, 2.0 mmol) were subsequently added into the solution of adenosine YD1-363 (153 mg, 0.4 mmol) in 10 mL acetone. The resulted mixture was stirred at room temperature overnight. After neutralization with saturated solution of sodium carbonate, the solution was extracted with EA (3×50 mL). The extracts were combined, washed with brine, dried with anhydrous sodium sulfate. The volatiles were removed under low pressure. The generated oil was separated with combiflash (DCM/MeOH, 10/1) to afford YD1-370 (132 mg, 78%).

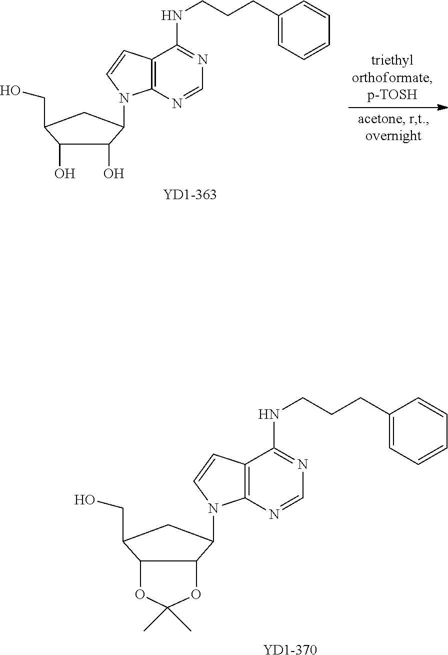 [SMILES: OCC1CC(n2ccc3c(NCCCc4ccccc4)ncnc32)C(O)C1O ; "YD1-363" ; CC1(C)OC2C(CO)CC(n3ccc4c(NCCCc5ccccc5)ncnc43)C2O1 ; "YD1-370"]

To a stirred solution of PPh3 (262 mg, 1.0 mmol, 2.0 eq) in anhydrous THE (10 mL) under nitrogen was added DIAD (202 mg, 1.0 mmol, 2.0 eq) at ice bath and stirred for 1 hour. Then, YD1-370 (211 mg, 0.5 mmol, 1.0 eq) was added and stirred for another 2 hours. Next, thiol acetic acid (56 mg, 0.75 mmol, 1.5 eq) was added. The reaction was slowly raised to room temperature and stirred for 24 hours. After the reaction was finished, the mixture was filtered and the filtrates was evaporated under vacuum. The generated oil was separated with combiflash (DCM/MeOH, 10/1) to afford YD1-371 (180 mg, 75%).

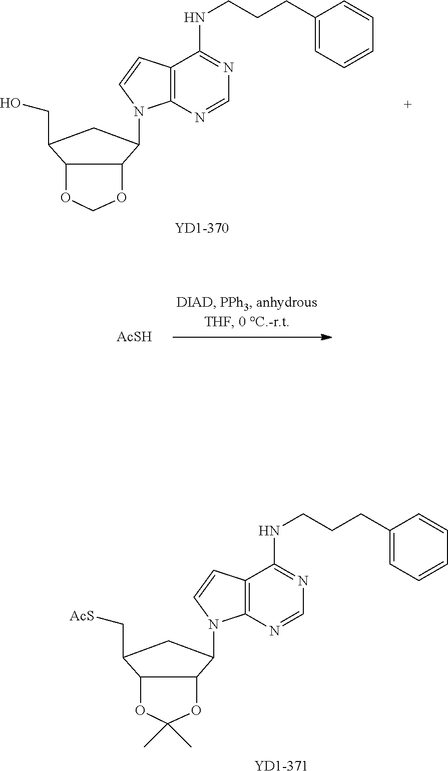

To a stirred solution of YD1-371 (240 mg, 0.5 mmol) in anhydrous MeOH (10 mL) under nitrogen was added tert-butyl (3-bromopropyl)carbamate (143 mg, 0.6 mmol) and sodium methoxide (30% in methanol, 539 μl, 3.0 mmol) at 0° C. The reaction mixture was stirred overnight. At the end of the reaction, acetic acid (0.5 ml) was added and the solvent was removed under vacuo. The residues were dissolved in 50 ml water and extracted with EA (3×50 mL). The extracts were combined, washed with water and dried with anhydrous sodium sulfate. The volatiles were removed under low pressure. The generated oil was separated with combiflash (DCM/MeOH, 10/1) to afford YD1-372 (241 mg, 81%).

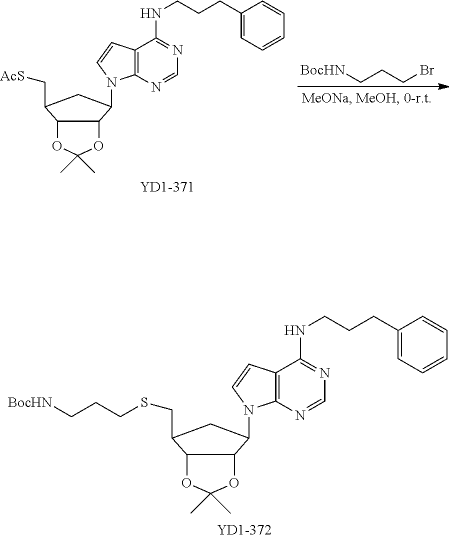

To a stirred solution of YD1-372 (238 mg, 0.4 mmol) in DCM (10 mL) was added 2 mL TFA. The reaction mixture was stirred for 4 h. At the end of the reaction, the volatiles were removed under vacuo. The residues were washed with cold Et2O (3×50 mL). The residues were dissolved in methanol and adjusted pH with 2M NaOH. After evaporation of the solvent, the crude product was washed with Hexane and directly used in the next step.

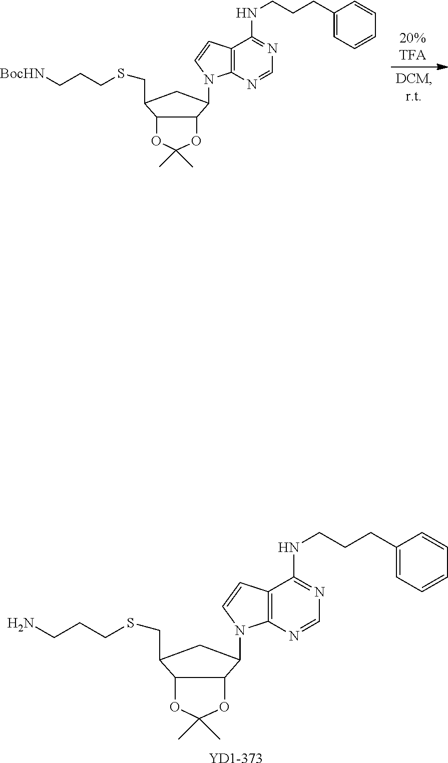

To a solution of Fmoc-thioisocynate (167 mg, 0.6 mmol) in 10 ml of DCM was slowly added 3-bromoaniline (103 mg, 0.6 mmol) in 10 ml DCM under ice bath. The mixture was stirred overnight at room temperature. The solvent was then disstiled to obtain a oil crude, wich was directly used for the next step.

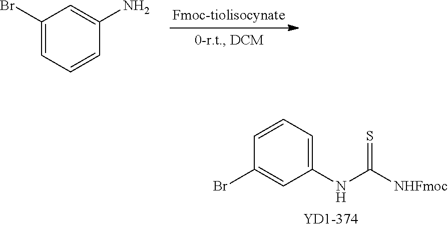

To a solution of YD1-373 (198 mg, 0.4 mmol) and YD1-374 (272 mg, 0.6 mmol) in anhydrous DCM/DMF (8/2 ml) was added DIPEA (155 mg, 1.2 mmol) and EDC (154 mg, 0.8 mmol) at room temperature. The reaction mixture was stirred overnight. At the end of the reaction, the mixture was diluted with EA and washed with brine. The solvent was removed under vacuo. The residue was dissolved in 10 ml DCM and 10 ml piperidine. The mixture was stirred for 2 h at room temperature. The solvent was removed under vacuo. The residues was washed with diethyl ether (2×30 ml) and dissolved in 10 ml of TFA containing 10% water, which was stirred at room temperature for 5 h. After that, the solvent was removed under nitrogen and dissolved in 2 ml MeOH, which was separated with preparative HPLC (MeCN/H₂O) to afford YD1-375 (107 mg, 41%).

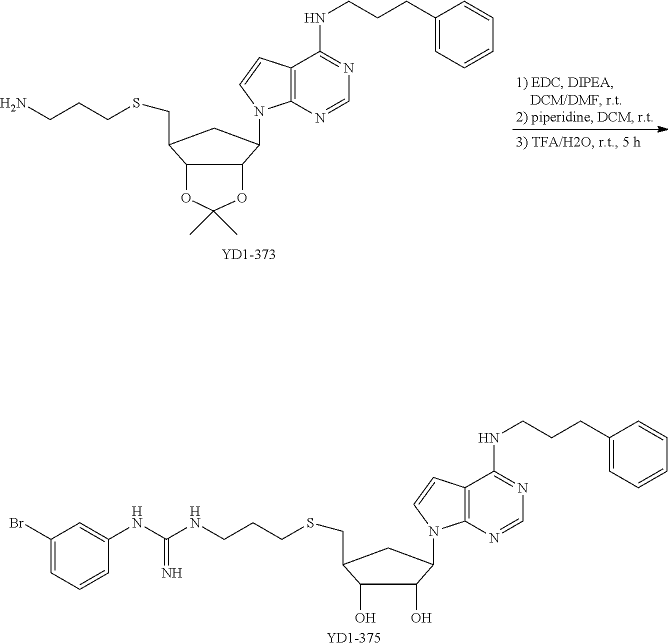

### Synthesis of YD247

A mixture of propargylamine (55.0 mg), 6-chloropurine riboside (143 mg, 0.5 mmol), and Et3N (2 mL, 12 mmol) in 1-propanol (25 mL) was heated at 70° C. for 6 h. The mixture was concentrated under reduced pressure, and trituratedn with H₂O to give white precipitates, which were filtered to yield YD1-242 (125 mg, 82%).

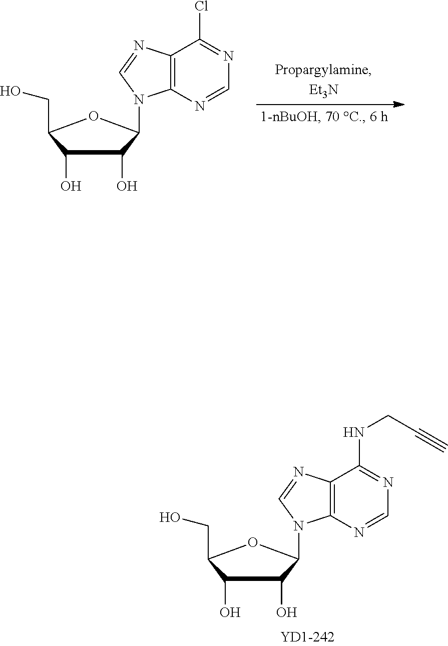

To a solution of PPh3 (2080.0 mg, 8.0 mmol, 2.0 eq) in anhydrous THE (50.0 mL) under nitrogen was added DIAD (1600.0 mg, 8.0 mmol, 2.0 eq) at ice bath and stirred for 1 hour. Then, YD1-242 (1220.0 mg, 4.0 mmol, 1.0 eq) was added and stirred for another 2 hours. Next, thiol acetic acid (464.0 mg, 6.0 mmol, 1.5 eq) was added. The reaction was slowly raised to room temperature and stirred for 24 hours. After the reaction was finished, the mixture was filtered and the filtrates was evaporated under vacuum. The crude product was dissolved in methanol and separated with pre-HPLC (Mobile phase, MeOH and water) to generate YD1-243.

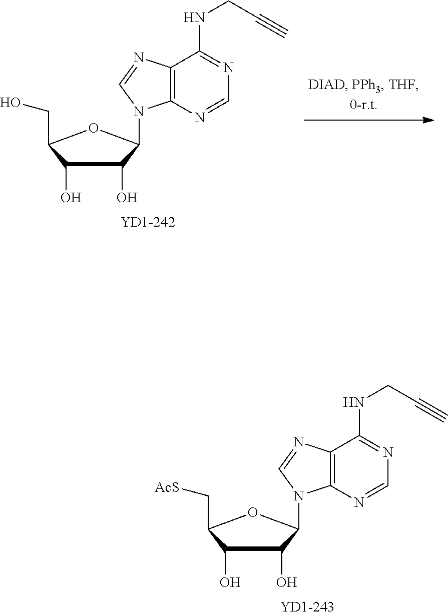

To a solution of YD1-08 (3250.0 mg, 10.0 mmol) and Bromoalkyl (2844.0 mg, 12 mmol) in anhydrous Methanol (100.0 ml) was added sodium methoxide (30% in methanol, 5390.0 μl, 30.0 mmol) at 0° C. The reaction mixture was stirred overnight. At the end of the reaction, acetic acid (4000 μml) was added and the solvent was removed under vacuo. The residues were dissolved in 200 ml water and extracted with EA (3×150 mL). The extracts were washed with water and dried with anhydrous sodium sulfate. After evaporation of the solvent, the crude product was washed with Hexane.

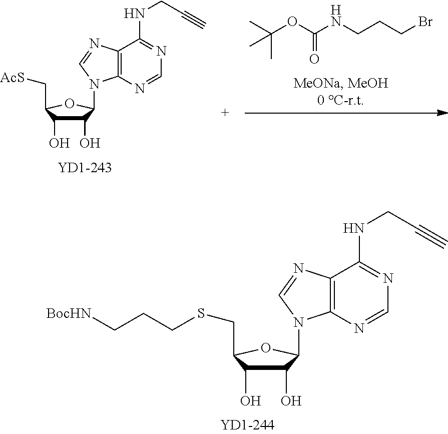

To a solution of Fmoc-thioisocynate (562.0 mg, 2.0 mmol) in 20 ml of DCM was slowly added 3-bromoaniline (344.0 mg, 2.0 mmol) in 10 ml DCM under ice bath. The mixture was stirred overnight at room temperature. The solvent was then disstiled to obtain a oil crude, wich was separated with silica gel (Hexane/EA, 20/1) to afford YD1-245 (654.0 mg, 83% yield).

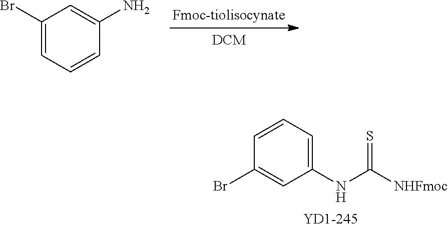

To a solution of YD1-244 (756.0 mg, 2.0 mmol) and YD1-245 (906.0 mg, 2.0 mmol) in anhydrous DCM/DMF (16/4 ml) was added DIPEA (387.0 mg, 3.0 mmol) and EDC (372.0 mg, 2.4 mmol) at room temperature. The reaction mixture was stirred overnight. At the end of the reaction, the mixture was diluted with EA and washed with brine. The solvent was removed under vacuo. The residue was dissolved in 10 ml DCM and 10 ml piperidine. The mixture was stirred for 2 h at room temperature. The solvent was removed under vacuo. The residues was washed with EA (2×30 ml) and dissolved in 4 ml of MeOH, which was separated with prep-HPLC (MeCN/water) to afford YD1-63.

### Synthesis of YD248

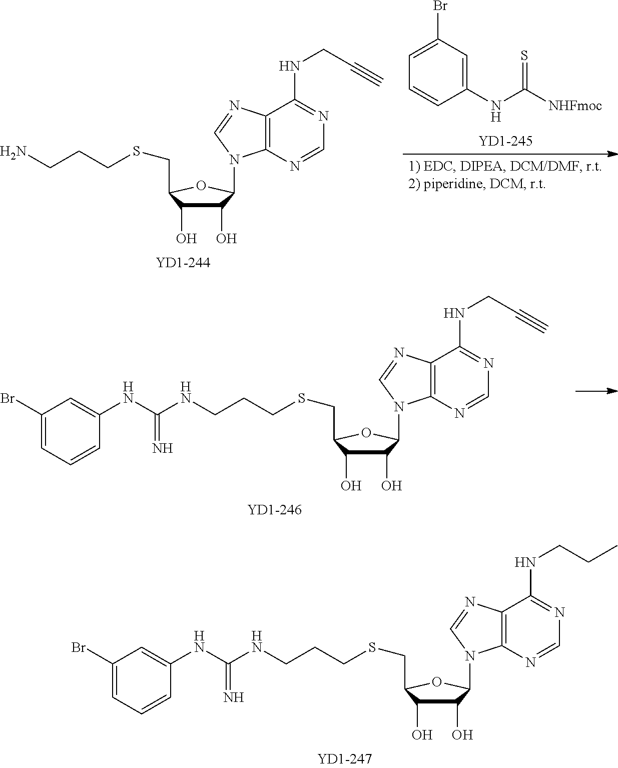

### Synthesis of YD248

To a solution of CuI (3.8 mg, 0.020 mmol), PdCl2(PPh3)2 (7.0 mg, 0.010 mmol), and the aryl iodide (45.0 mg, 0.22 mmol) in THF/Et3N (4/1, 0.45 M) was added under nitrogen. Then, YD1-246 (114.0 mg, 0.2 mmol) was added and the mixture was stirred overnight at rt. A saturated NH4Cl aqueous solution was added to the reaction mixture upon completion, then extracted with Et2O three times. The combined organic layer was washed with brine and dried over anhydrous Na2SO4, filtered, and concentrated under reduced pressure. The residue was purified by silica gel column chromatography (PE/EA/Et3N=2/1/1%). The corresponding propargyl amines were obtained in high yields.

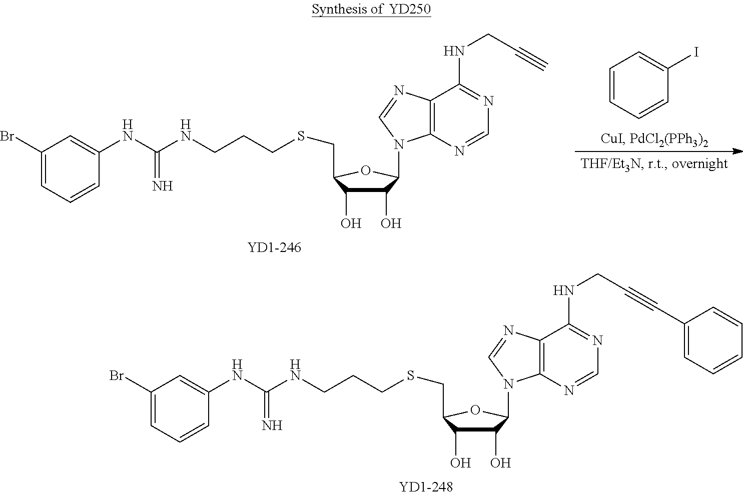

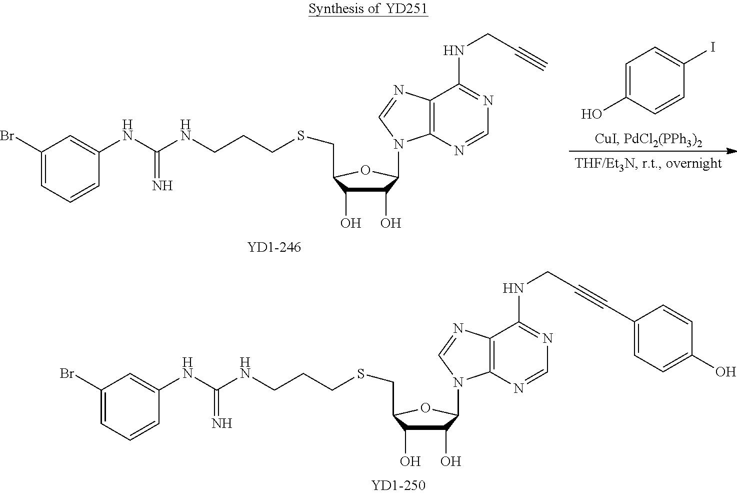

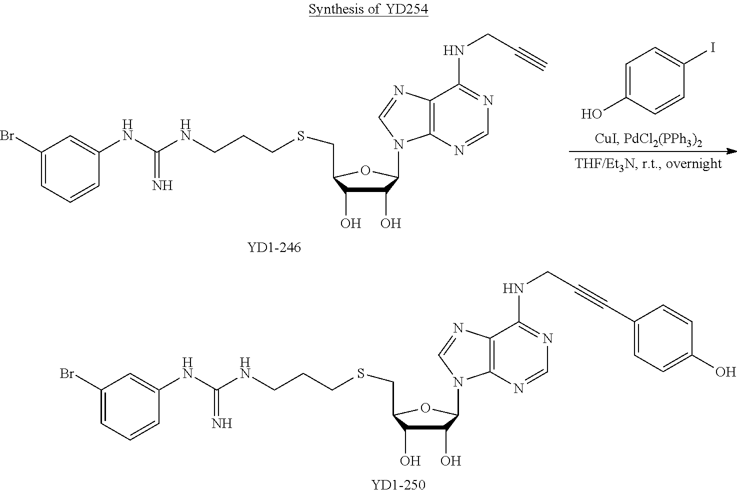

To a stirred solution of NaN3 (71.5 mg, 1.1 mmol) in DMSO (2 mL) was added bromoalkane (1 mmol). The reaction mixture was stirred at 80° C. overnight. Then the reaction mixture was cooled to room temperature and diluted with water (5 mL). The mixture was extracted with ether (3×5 mL) and washed by brine, dried over Na2SO4 and concentrated under vacuum to give the products in quantitative yields. It was used directly without further purification.

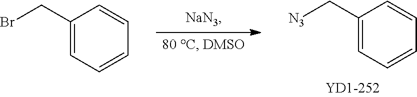 [SMILES: BrCc1ccccc1 ; [N-]=[N+]=NCc1ccccc1]

To a mixture of YD1-246 (115.0 mg, 0.2 mmol) and benzyl azide (39.0 mg, 0.3 mmol) in EtOH (14 mL) was added a solution of CuSO4 (8.0 mg, 0.05 mmol), (+) sodium L-ascorbate (15.0 mg, 0.075 mmol) and K2CO3 (28.0 mg, 0.2 mmol) in H₂O (36 mL). After the mixture was stirred at room temperature for 15 h, the mixture was then concentrated and extracted with DCM (3×50 mL). The combined extracts were washed with brine, dried over anhydrous sodium sulfate, filtered, and concentrated. Crystallization of the crude residue by using solvent system with n-hexane gave the product Linker 1-II (7.88 g, 83%).

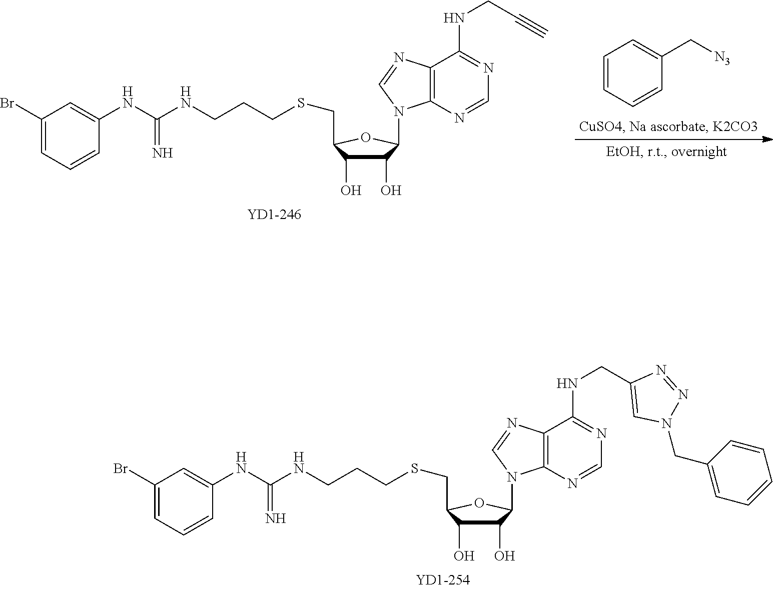

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A compound having a formula (I): 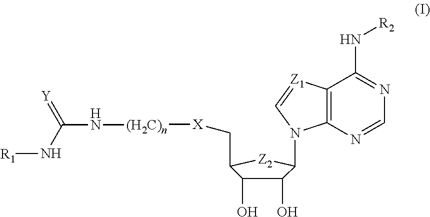
or a pharmaceutically acceptable salt thereof, wherein:
X is a N or S,
n is an integer from 1-12; and
Y is NR^{p} or O, wherein R^{p} is H or a protecting group, and
Z₁ is N or CH, and
Z₂ is O or CH₂, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl.

2. (canceled)

3. (canceled)

4. (canceled)

5. (canceled)

6. The compound of claim 1, or a pharmaceutically acceptable salt thereof, wherein X is S and/or Z₁ is N.

7. (canceled)

8. (canceled)

9. (canceled)

10. The compound of claim 1, or a pharmaceutically acceptable salt thereof, wherein R₁ and/or R₂ is selected from the group consisting of:

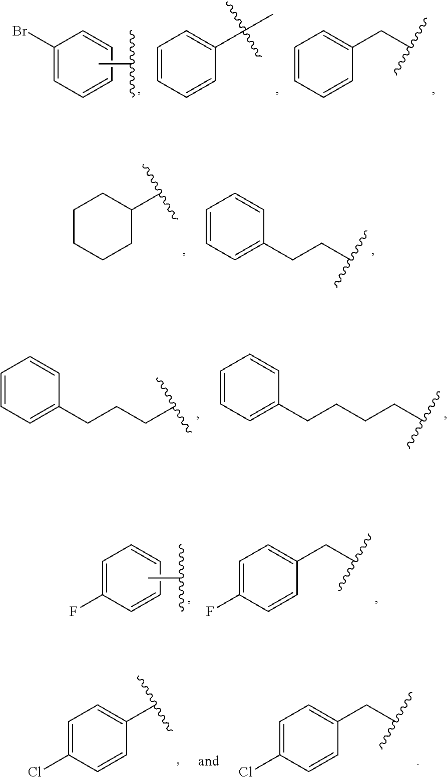

11. The compound of claim 1, wherein the compound has the formula: 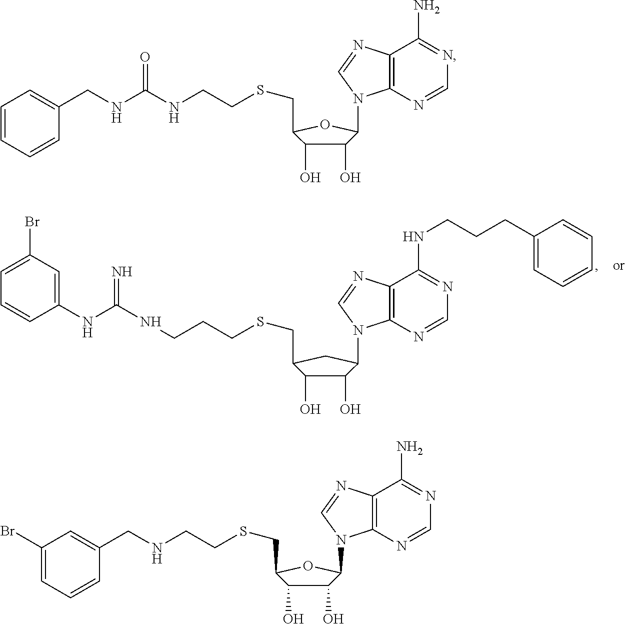 [SMILES: Nc1ncnc2c1ncn2C1OC(CSCCNC(=O)NCc2ccccc2)C(O)C1O ; N=C(NCCCSCC1CC(n2cnc3c(NCCCc4ccccc4)ncnc32)C(O)C1O)Nc1cccc(Br)c1 ; Nc1ncnc2c1ncn2[C@@H]1O[C@H](CSCCNCc2cccc(Br)c2)[C@@H](O)[C@H]1O]
or a pharmaceutically acceptable salt thereof.

12. The compound of claim 1, wherein the compound has a formula (II): 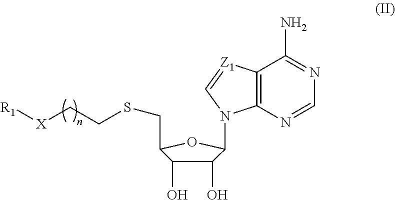
or a pharmaceutically acceptable salt thereof,
wherein:
n is an integer from 0-12,
Z₁ is N or CH, and
X is O or NH and
R₁ is an aryl or heterocyclic, R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl; or 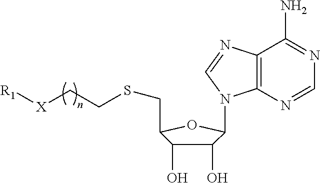
or a pharmaceutically acceptable salt thereof,
wherein:
n is an integer from 0-12,
X is O or NH and
R₁ is an aryl or heterocyclic.

13. (canceled)

14. The compound of claim 12, wherein the compound has the formula: 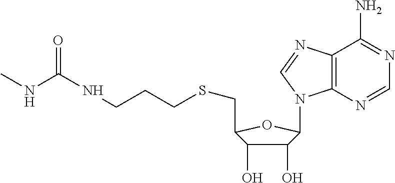 [SMILES: CNC(=O)NCCCSCC1OC(n2cnc3c(N)ncnc32)C(O)C1O]
or a pharmaceutically acceptable salt thereof.

15. A compound having the formula (III), wherein the compound has the formula (III): 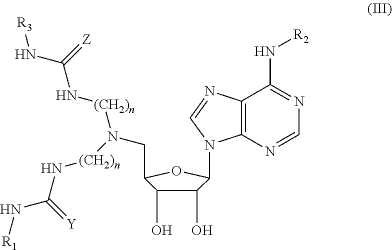
or a pharmaceutically acceptable salt thereof,
wherein:
each n is independently an integer from 1-12; and
Y and Z are either NH or O, and
R₁ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl, and
R₃ is an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl.

16. (canceled)

17. The compound of claim 15, wherein the compound has the formula: 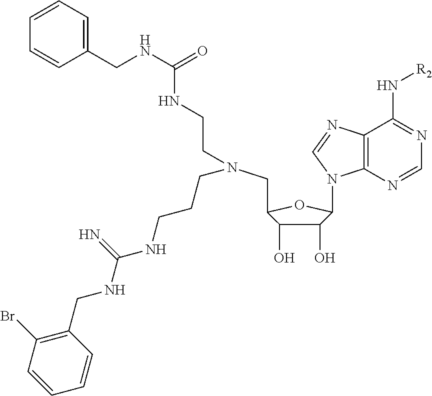
or a pharmaceutically acceptable salt thereof.

18. (canceled)

19. The compound of claim 15, wherein Y is NH.

20. The compound of claim 15, wherein Z is O and/or Y is O; or wherein Y is O and/or Z is NH.

21. (canceled)

22. (canceled)

23. The compound of claim 15, wherein R₁ and R₃ are each, independently: 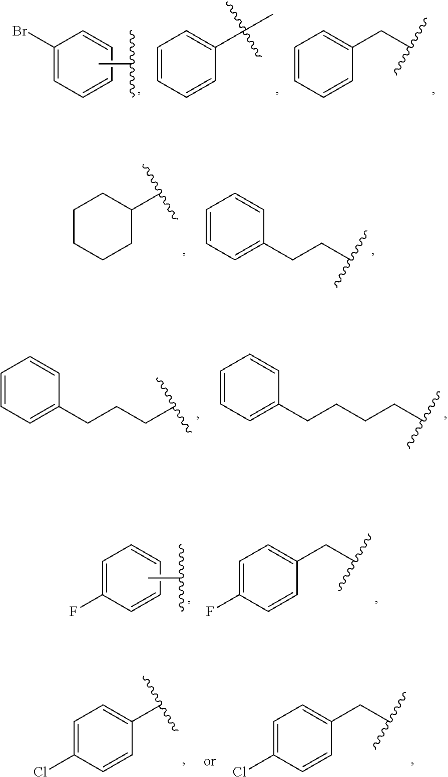
each of which can be further substituted.

24. (canceled)

25. (canceled)

26. (canceled)

27. (canceled)

28. (canceled)

29. A compound of the formula: 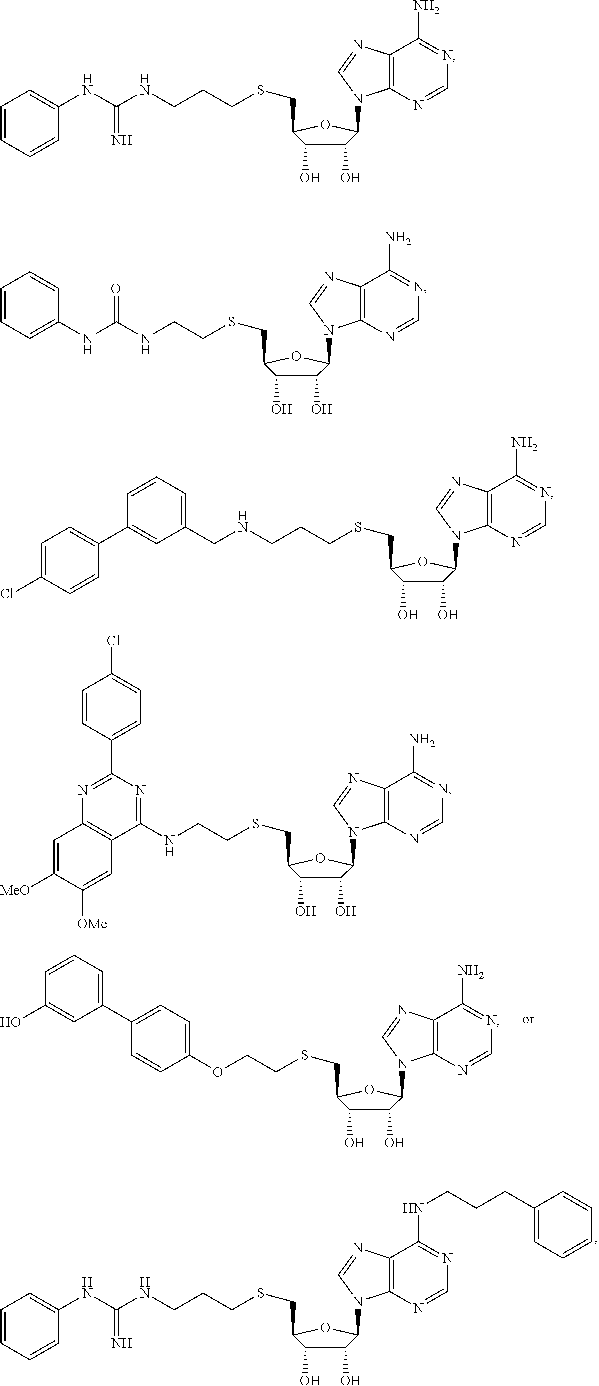 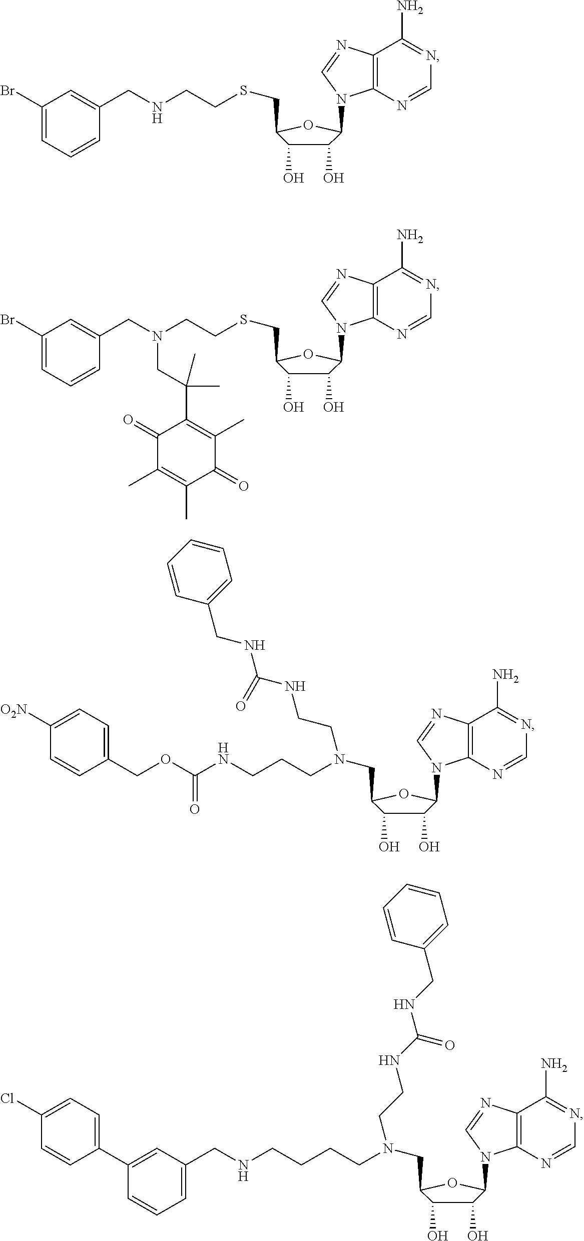 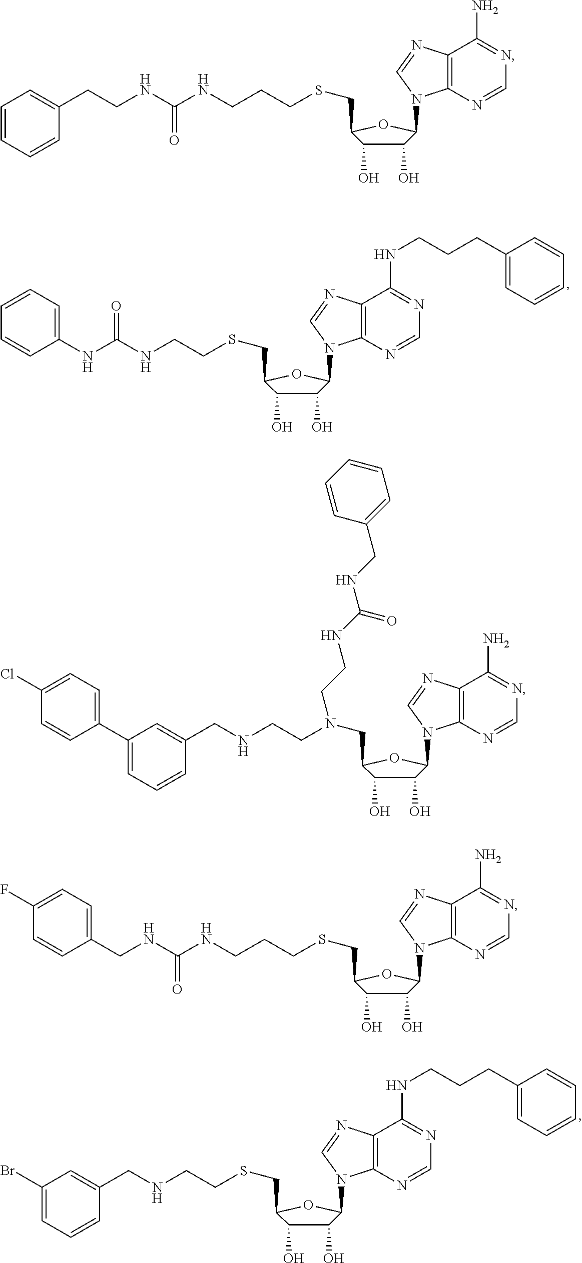 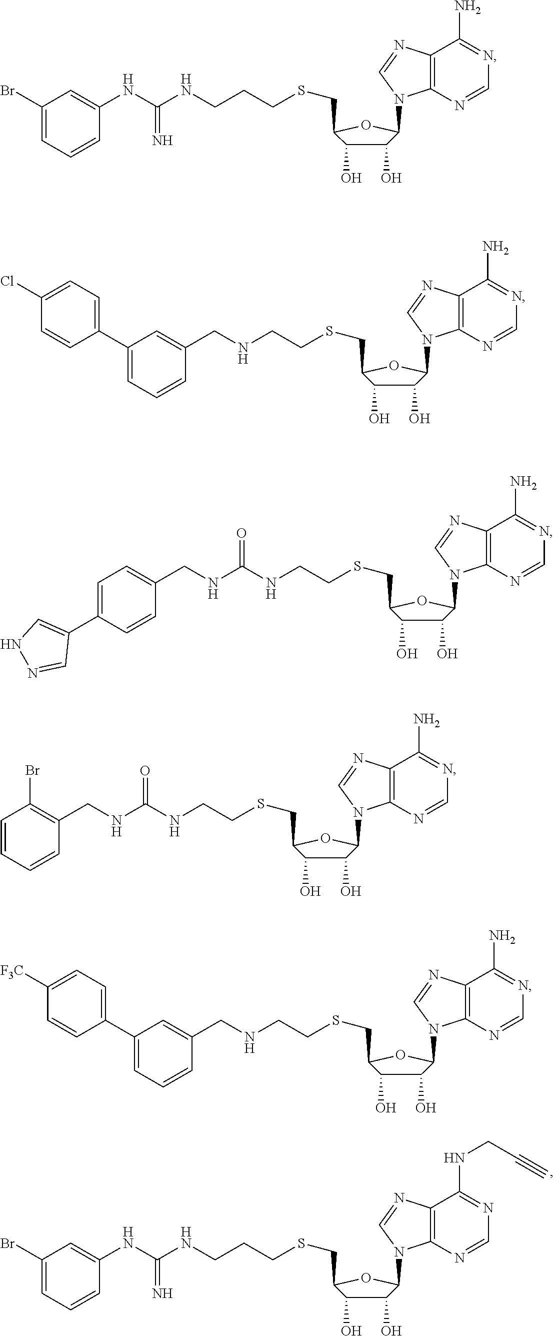 [SMILES: N=C(NCCCSC[C@H]1O[C@@H](n2cnc3c(N)ncnc32)[C@H](O)[C@@H]1O)Nc1cccc(Br)c1 ; Nc1ncnc2c1ncn2[C@@H]1O[C@H](CSCCNCc2cccc(-c3ccc(Cl)cc3)c2)[C@@H](O)[C@H]1O ; Nc1ncnc2c1ncn2[C@@H]1O[C@H](CSCCNC(=O)NCc2ccc(-c3cn[nH]c3)cc2)[C@@H](O)[C@H]1O ; Nc1ncnc2c1ncn2[C@@H]1O[C@H](CSCCNC(=O)NCc2ccccc2Br)[C@@H](O)[C@H]1O ; Nc1ncnc2c1ncn2[C@@H]1O[C@H](CSCCNCc2cccc(-c3ccc(C(F)(F)F)cc3)c2)[C@@H](O)[C@H]1O ; C#CCNc1ncnc2c1ncn2[C@@H]1O[C@H](CSCCCNC(=N)Nc2cccc(Br)c2)[C@@H](O)[C@H]1O] 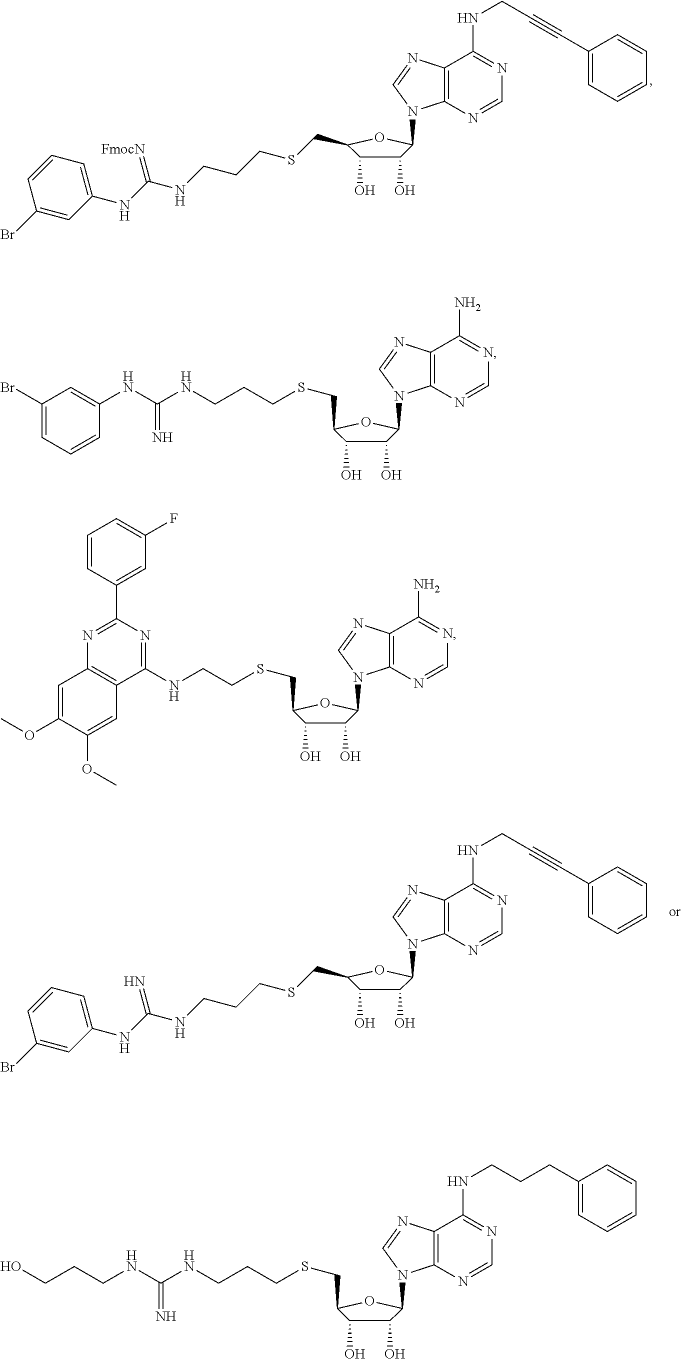
or pharmaceutically acceptable salt thereof.

30. A compound of the formula: 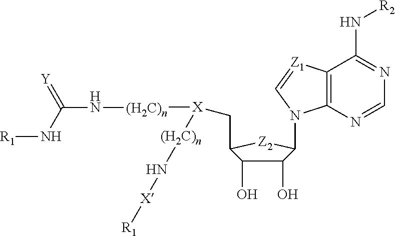
or a pharmaceutically acceptable salt thereof, wherein:
X is a N or S,
X′ is a NH, S or O,
n is an integer from 1-12; and
Y is NR^{p} or O, wherein R^{p} is H or a protecting group, and
Z₁ is N or CH, and
Z₂ is O or CH₂, and
each R₁ is, independently, an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, or arylalkenyl, and
R₂ is an H or an acyl, alkyl, alkenyl, alkynyl, heteroalkyl, heteroalkenyl, heteroalkynyl, cycloalkyl, heterocyloalkyl, cycloalkenyl, heterocycloakenyl, heterocyclyl, or an optionally substituted aryl, arylalkyl, arylalkenyl or aryalkynyl.

31. (canceled)

32. (canceled)

33. (canceled)

34. (canceled)

35. The compound of claim 30, or a pharmaceutically acceptable salt thereof, wherein X is S and/or Z₁ is N.

36. (canceled)

37. (canceled)

38. (canceled)

39. The compound of claim 30, or a pharmaceutically acceptable salt thereof, wherein each R₁ and/or R₂ is independently selected from the group consisting of:

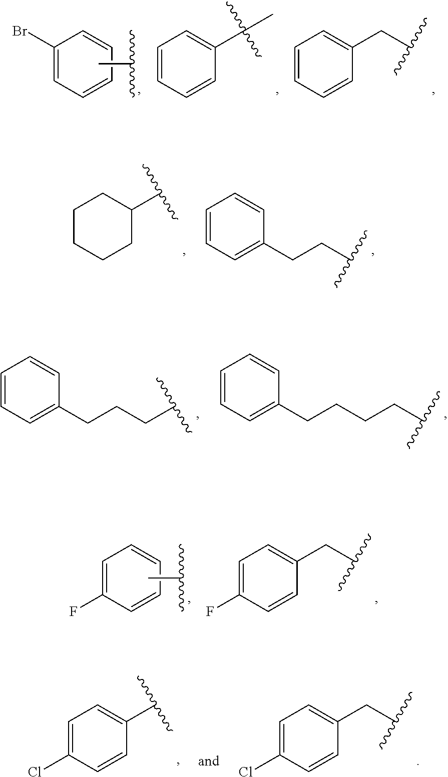

40. The compound of claim 30, or a pharmaceutically acceptable salt thereof, wherein the compound is: 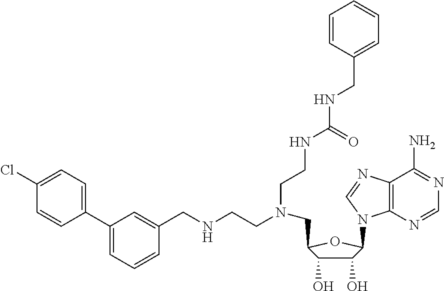 [SMILES: Nc1ncnc2c1ncn2[C@@H]1O[C@H](CN(CCNCc2cccc(-c3ccc(Cl)cc3)c2)CCNC(=O)NCc2ccccc2)[C@@H](O)[C@H]1O]
or a pharmaceutically acceptable salt thereof.

41. A pharmaceutical composition comprising one or more compounds of claim 1, or a pharmaceutically acceptable salt thereof, together with one or more diluents, excipients or carriers.

42. (canceled)

43. A method for inhibiting PRMT activity comprising the step of administering to a mammal in need of relief from the effects of PRMT activity a therapeutically effective amount of one or more compounds of claim 1 or a pharmaceutically acceptable salt thereof.

44. (canceled)

45. The method of claim 43, wherein the one or more compounds is selective for at least one of PRMT1, PRMT4, and PRMT5.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. The method of claim 43, wherein the compound that is administered is YD1130, YD1113, YD1290, YD1349, YD1195, AK442 or YD1133.

59. (canceled)

60. (canceled)

61. (canceled)

62. (canceled)

63. (canceled)

64. (canceled)
